(19) United States
(12) Patent Application Publication
(10) Pub. No.: US 2023/0330245 A1
(43) Pub. Date: Oct. 19, 2023

(54) ANTIBODY-EXATECAN CONJUGATES

(71) Applicant: Synaffix B.V., Oss (NL)

(72) Inventors: Jorin HOOGENBOOM, Wageningen (NL); Arnoldus Jacobus VAN SCHAIK, Oss (NL); Sorraya POPAL, Oss (NL); Laureen DE BEVER, Oss (NL); Maria Antonia WIJDEVEN, Oss (NL); Remon VAN GEEL, Lithoijen (NL); Inge Catharina Josephina HURKMANS, Liessel (NL); Peter VAN DE SANDE, Oss (NL); Sander Sebastiaan VAN BERKEL, Oss (NL); Floris Louis VAN DELFT, Nijmegen (NL)

(21) Appl. No.: 18/121,447

(22) Filed: Mar. 14, 2023

Related U.S. Application Data
(63) Continuation of application No. PCT/EP2021/075401, filed on Sep. 15, 2021.

(30) Foreign Application Priority Data
Sep. 15, 2020 (EP) 20196331.1
Sep. 17, 2020 (EP) 20196784.1

(51) Int. Cl. A61K 47/68; C07K 16/32; A61P 35/00

(52) U.S. Cl. CPC A61K 47/68037; C07K 16/32; A61K 47/6855; A61P 35/00

(57) ABSTRACT

The present invention concerns an antibody-drug conjugate, having structure (1)

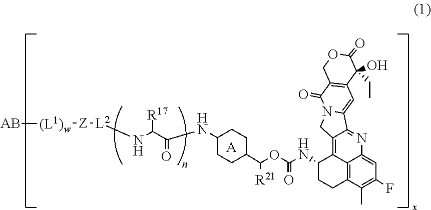

wherein AB is an antibody; L¹ and L² are linkers; w is 0 or 1; Z is a connecting group obtained by a metal-free click reaction or by thiol ligation; each R¹⁷ is individually an amino acid side chain; n is an integer in the range of 1-5; A is a 5- or 6-membered aromatic or heteroaromatic ring; x is an integer in the range of 1-8; R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups.

### CROSS-REFERENCE TO RELATED APPLICATIONS

This application is a continuation of International Patent Application No. PCT/EP2021/075401 filed Sep. 15, 2021, which application claims priority to European Patent Application No. 20196331.1 filed Sep. 15, 2020, and to European Patent Application No. 20196784.1 filed Sep. 17, 2020, the contents of which are all incorporated herein by reference in their entireties.

### SEQUENCE LISTING

A sequence listing in electronic ST.26 XML format is filed with this application and incorporated herein by reference. The name of the XML file is “069818-1080_ST26_SL”; the file was created on Jun. 9, 2023; the size of the file is 2.08 KB.

### FIELD OF THE INVENTION

The present invention relates to the field of antibody-drug conjugates, in particular to antibody-drug conjugates with an exatecan cytotoxic payload, which are suitable for the treatment of cancer.

### BACKGROUND OF THE INVENTION

Antibody-drug conjugates (ADC), considered as magic bullets in therapy, are comprised of an antibody to which is attached a pharmaceutical agent. The antibodies (also known as ligands) can be small protein formats (scFv's, Fab fragments, DARPins, Affibodies, etc.) but are generally monoclonal antibodies (mAbs) which have been selected based on their high selectivity and affinity for a given antigen, their long circulating half-lives, and little to no immunogenicity. Thus, mAbs as protein ligands for a carefully selected biological receptor provide an ideal delivery platform for selective targeting of pharmaceutical drugs. For example, a monoclonal antibody known to bind selectively with a specific cancer-associated antigen can be used for delivery of a chemically conjugated cytotoxic agent to the tumour, via binding, internalization, intracellular processing and finally release of active catabolite. The cytotoxic agent may be small molecule toxin, a protein toxin or other formats, like oligonucleotides. As a result, the tumour cells can be selectively eradicated, while sparing normal cells which have not been targeted by the antibody. Similarly, chemical conjugation of an antibacterial drug (antibiotic) to an antibody can be applied for treatment of bacterial infections, while conjugates of anti-inflammatory drugs are under investigation for the treatment of autoimmune diseases and for example attachment of an oligonucleotide to an antibody is a potential promising approach for the treatment of neuromuscular diseases. Hence, the concept of targeted delivery of an active pharmaceutical drug to a specific cellular location of choice is a powerful approach for the treatment of a wide range of diseases, with many beneficial aspects versus systemic delivery of the same drug.

An alternative strategy to employ monoclonal antibodies for targeted delivery of a specific protein agent is by genetic fusion of the latter protein to one (or more) of the antibody's termini, which can be the N-terminus or the C-terminus on the light chain or the heavy chain (or both). In this case, the biologically active protein of interest, e.g. a protein toxin like Pseudomonas exotoxin A (PE38) or an anti-CD3 single chain variable fragment (scFv), is genetically encoded as a fusion to the antibody, possibly but not necessarily via a peptide spacer, so the antibody is expressed as a fusion protein. The peptide spacer may contain a protease-sensitive cleavage site, or not.

In the field of ADCs, a chemical linker is typically employed to attach a pharmaceutical drug to an antibody. This linker needs to possess a number of key attributes, including the requirement to be stable in plasma after drug administration for an extended period of time. A stable linker enables localization of the ADC to the projected site or cells in the body and prevents premature release of the payload in circulation, which would indiscriminately induce undesired biological response of all kinds, thereby lowering the therapeutic index of the ADC. Upon internalization, the ADC should be processed such that the payload is effectively released so it can bind to its target.

There are two families of linkers, non-cleavable and cleavable. Non-cleavable linkers consist of a chain of atoms between the antibody and the payload, which is fully stable under physiological conditions, irrespective of which organ or biological compartment the antibody-drug conjugate resides in. As a consequence, liberation of the payload from an ADC with a non-cleavable linker relies on the complete (lysosomal) degradation of the antibody after internalization of the ADC into a cell. As a consequence of this degradation, the payload will be released, still carrying the linker, as well as a peptide fragment and/or the amino acid from the antibody the linker was originally attached to. Cleavable linkers utilize an inherent property of a cell or a cellular compartment for selective release of the payload from the ADC, which generally leaves no trace of linker after metabolic processing. For cleavable linkers, there are three commonly used mechanisms: 1) susceptibility to specific enzymes, 2) pH-sensitivity, and 3) sensitivity to redox state of a cell (or its microenvironment). The cleavable linker may also contain a self-immolative unit, for example based on a para-aminobenzyl alcohol group and derivatives thereof. A linker may also contain an additional, non-functional element, often referred to as spacer or stretcher unit, to connect the linker with a reactive group for reaction with the antibody.

Although ADCs have demonstrated clinical and preclinical activity, it has been unclear what factors determine such potency in addition to antigen expression on targeted tumour cells. For example, drug:antibody ratio (DAR), ADC-binding affinity, potency of the payload, receptor expression level, internalization rate, trafficking, multiple drug resistance (MDR) status, and other factors have all been implicated to influence the outcome of ADC treatment in vitro. In addition to the direct killing of antigen-positive tumour cells, ADCs also have the capacity to kill adjacent antigen-negative tumour cells: the so-called “bystander killing” effect, as originally reported by Sahin et al, Cancer Res. 1990, 50, 6944-6948, incorporated by reference, and for example studied by Li et al, Cancer Res. 2016, 76, 2710-2719, incorporated by reference. Generally spoken, cytotoxic payloads that are neutral will show bystander killing whereas ionic (charged) payloads do not, as a consequence of the fact that ionic species do not readily pass a cellular membrane by passive diffusion. Payloads with established bystander effect are for example MMAE and DXd. Examples of payloads that do not show bystander killing are MMAF or the active catabolite of Kadcyla (lysine-MCC-DM1).

ADCs are prepared by conjugation of a linker-drug with a protein, a process known as bioconjugation. Many technologies are known for bioconjugation, as summarized in G. T. Hermanson, “Bioconjugate Techniques”, Elsevier, 3^{rd} Ed. 2013, incorporated by reference. Two main technologies can be recognized for the preparation of ADCs by random conjugation, either based on acylation of lysine side chain or based on alkylation of cysteine side chain. Acylation of the s-amino group in a lysine side-chain is typically achieved by subjecting the protein to a reagent based on an activated ester or activated carbonate derivative, for example SMCC is applied for the manufacturing of Kadcyla®. Main chemistry for the alkylation of the thiol group in cysteine side-chain is based on the use of maleimide reagents, as is for example applied in the manufacturing of Adcetris®. Besides standard maleimide derivatives, a range of maleimide variants are also applied for more stable cysteine conjugation, as for example demonstrated by James Christie et al., J. Contr. Rel. 2015, 220, 660-670 and Lyon et al., Nat. Biotechnol. 2014, 32, 1059-1062, both incorporated by reference. Other approaches for cysteine alkylation involve for example nucleophilic substitution of haloacetamides (typically bromoacetamide or iodoacetamide), see for example Alley et al., Bioconj. Chem. 2008, 19, 759-765, incorporated by reference, or various approaches based on nucleophilic addition on unsaturated bonds, such as reaction with acrylate reagents, see for example Bernardim et al., Nat. Commun. 2016, 7, DOI: 10.1038/ncomms13128 and Ariyasu et al., Bioconj. Chem. 2017, 28, 897-902, both incorporated by reference, reaction with phosphonamidates, see for example Kasper et al., Angew. Chem. Int. Ed. 2019, 58, 11625-11630, incorporated by reference, reaction with allenamides, see for example Abbas et al., Angew. Chem. Int. Ed. 2014, 53, 7491-7494, incorporated by reference, reaction with cyanoethynyl reagents, see for example Kolodych et al., Bioconj. Chem. 2015, 26, 197-200, incorporated by reference, reaction with vinylsulfones, see for example Gil de Montes et al., Chem. Sci. 2019, 10, 4515-4522, incorporated by reference, or reaction with vinylpyridines, see for example https://iksuda.com/science/permalink/ (accessed Jan. 7, 2020). An alternative approach to antibody conjugation without reengineering of antibody involves the reduction of interchain disulfide bridges, followed addition of a payload attached to a cysteine cross-linking reagent, such as bis-sulfone reagents, see for example Balan et al., Bioconj. Chem. 2007, 18, 61-76 and Bryant et al., Mol. Pharmaceutics 2015, 12, 1872-1879, both incorporated by reference, mono- or bis-bromomaleimides, see for example Smith et al., J. Am. Chem. Soc. 2010, 132, 1960-1965 and Schumacher et al., Org. Biomol. Chem. 2014, 37, 7261-7269, both incorporated by reference, bis-maleimide reagents, see for example WO2014114207, bis(phenylthio)maleimides, see for example Schumacher et al., Org. Biomol. Chem. 2014, 37, 7261-7269 and Aubrey et al., Bioconj. Chem. 2018, 29, 3516-3521, both incorporated by reference, bis-bromopyridazinediones, see for example Robinson et al., RSC Advances 2017, 7, 9073-9077, incorporated by reference, bis(halomethyl)benzenes, see for example Ramos-Tomillero et al., Bioconj. Chem. 2018, 29, 1199-1208, incorporated by reference or other bis(halomethyl)aromatics, see for example WO2013173391. Typically, ADCs prepared by cross-linking of cysteines have a drug-to-antibody loading of ˜4 (DAR4). Another useful technology for conjugation to a cysteine side chain is by means of disulfide bond, a bioactivatable connection that has been utilized for reversibly connecting protein toxins, chemotherapeutic drugs, and probes to carrier molecules (see for example Pillow et al., Chem. Sci. 2017, 8, 366-370, incorporated by reference).

Besides conjugation to lysine or cysteine, a range of other conjugation technologies has been explored in the past decade. One method is based on genetic encoding of a non-natural amino acid, e.g. p-acetophenylalanine suitable for oxime ligation, or p-azidomethylphenylalanine or p-azidophenylalanine suitable for click chemistry conjugation, as for example demonstrated by Axup et al. Proc. Nat. Acad. Sci. 2012, 109, 16101-16106, incorporated by reference. Similarly, Zimmerman et al., Bioconj. Chem. 2014, 25, 351-361, incorporated by reference have employed a cell-free protein synthesis method to introduce azidomethylphenylalanine (AzPhe) into monoclonal antibodies for conversion into ADC by means of metal-free click chemistry. Also, it has also be shown by Nairn et al., Bioconj. Chem. 2012, 23, 2087-2097, incorporated by reference, that a methionine analogue like azidohomoalanine (Aha) can be introduced into protein by means of auxotrophic bacteria and further converted into protein conjugates by means of (copper-catalysed) click chemistry. Finally, genetic encoding of aliphatic azides in recombinant proteins using a pyrrolysyl-tRNA synthetase/tRNAcUA pair was shown by Nguyen et al., J. Am. Chem. Soc. 2009, 131, 8720-8721, incorporated by reference and labelling was secured by click chemistry.

Another method is based on enzymatic installation of a non-natural functionality. For example, Lhospice et al., Mol. Pharmaceut. 2015, 12, 1863-1871, incorporated by reference, employ the bacterial enzyme transglutaminase (BTG or TGase) for installation of an azide moiety onto an antibody. A genetic method based on C-terminal TGase-mediated azide introduction followed by conversion in ADC with metal-free click chemistry was reported by Cheng et al., Mol. Cancer Therap. 2018, 17, 2665-2675, incorporated by reference.

It has been shown in WO2014065661, by van Geel et al., Bioconj. Chem. 2015, 26, 2233-2242 and Verkade et al., Antibodies 2018, 7, 12, all incorporated by reference, that enzymatic remodelling of the native antibody glycan at N297 enables introduction of an azido-modified sugar, suitable for attachment of cytotoxic payload using click chemistry (see FIG. 5A). Alternatively, a modified sugar is enzymatically incorporated harbouring a disulfide linkage, which upon subsequent reduction is liberated to give a free thiol group for conjugation by alkylation (see FIG. 5B).

Chemical approaches have also been developed for site-specific modification of antibodies without prior genetic modification, as for example highlighted by Yamada and Ito, ChemBioChem. 2019, 20, 2729-2737.

A frequent method for bioconjugation of linker-drugs to azido-modified proteins is strain-promoted alkyne-azide cycloaddition (SPAAC). In a SPAAC reaction, the linker-drug is functionalized with a cyclic alkyne and the cycloaddition with azido-modified antibody is driven by relief of ring-strain. Conversely, the linker-drug is functionalized with azide and the antibody with cyclic alkyne. Various strained alkynes suitable for metal-free click chemistry are indicated in FIG. 1A.

Besides with strained alkynes, bioconjugation of linker-drugs to antibodies (and other biomolecules such as glycans, nucleic acids) can be achieved by a range of other metal-free click chemistries, see e.g. Nguyen and Prescher, Nature rev. 2020, doi: 10.1038/s41570-020-0205-0, incorporated by reference. For example, oxidation of a specific tyrosine in a protein can give an ortho-quinone, which readily undergoes cycloaddition with strained alkenes (e.g. TCO) or strained alkynes, see e.g. Bruins et al., Chem. Eur. J. 2017, 24, 4749-4756, incorporated by reference. Besides cyclooctyne, certain cycloheptynes are also suitable for metal-free click chemistry, as reported by Wetering et al. Chem. Sci. 2020, doi: 10.1039/d0sc03477k, incorporated by reference. A tetrazine moiety can also be introduced into a protein or a glycan by various means, for example by genetic encoding or chemical acylation, and may also undergo cycloaddition with cyclic alkenes and alkynes. A list of couples of functional groups F and Q for metal-free click chemistry is provided in FIG. 2.

Based on the above, a general method for the preparation of a protein conjugate, exemplified for a monoclonal antibody in FIG. 3, entails the reaction of a protein containing x number of reactive moieties F with a linker-drug construct containing a single molecule Q. A schematic depiction how reactive molecules F can be introduced into a monoclonal antibody is provided in FIG. 4.

Conjugation of a cytotoxic payload to an antibody by any of the methods described above is often challenging due to the hydrophobic nature of the payload and in some cases in combination with the linker, which encumbers solubility in aqueous or buffered systems (the preferred medium for antibodies). As a consequence, conjugation of cytotoxic payload is typically performed in medium consisting of water/buffer plus an organic co-solvent. Typical co-solvents for conjugation are DMSO, propylene glycol (PG), ethanol, DMF, DMA and NMP, which facilitate solubilization of linker-drug but can also mix well with water. Typical amount of co-solvent is 10-25% versus aqueous medium, however, co-solvents may be added up to 50% in some cases. Adding high amount of co-solvent is particularly favourable for conjugation processes where the payload is significantly hydrophobic (lipophilic) and in those processes where a large excess of linker-drug is required to achieve full conversion to desired product.

Besides the apparent benefits, the downside of adding a significant amount of organic co-solvent is that the antibody may not be stable in the solvent mixture and as a consequence may aggregate during the conjugation process. Typically, aggregation levels will be correlated to the amount of co-solvent, but this is also antibody-dependent. Especially for unstable antibodies, aggregation levels may be significant, reaching levels of 10% or even more, which will consequently compromise process yields. Moreover, these levels of aggregates will require an additional processing step (e.g. SEC or CHT) to remove aggregates to an acceptable level. An additional disadvantage of high co-solvent levels during conjugation is the necessity to introduce an additional process step to remove the excess co-solvent, e.g. by dialysis, by spin-filtration or by TFF, before size-exclusion purification can be performed (SEC).

Currently, cytotoxic payloads include for example microtubule-disrupting agents [e.g. auristatins such as monomethyl auristatin E (MMAE) and monomethyl auristatin F (MMAF), maytansinoids, such as DM1 and DM4, tubulysins], DNA-damaging agents [e.g., calicheamicin, pyrrolobenzodiazepine (PBD) dimers, indolinobenzodiapine dimers, duocarmycins, anthracyclines], topoisomerase inhibitors [e.g. DXd, SN-38] or RNA polymerase II inhibitors [e.g. amanitin]. ADCs that have reached market approval include for example payloads MMAE, MMAF, DM1, calicheamicin, SN-38 and DXd, while various pivotal trials are running for ADCs based on duocarmycin, DM4 and PBD dimer. A larger variety of payloads is still under clinical evaluation or has been in clinical trials in the past, e.g. eribulin, indolinobenzodiazepine dimer, PNU-159,682, hemi-asterlin, doxorubicin, vinca alkaloids and others. Finally, various ADCs in late-stage preclinical stage are conjugated to novel payloads for example amanitin, KSP inhibitors, MMAD, and others.

With the exception of sacituzumab govetican (Trodelvy®), all of the clinical and marketed ADCs contain cytotoxic drugs that are not suitable as stand-alone drug. Trodelvy® is the exception because it features SN-38 as cytotoxic payload, which is also the active catabolite of irinotecan (an SN-38 prodrug). Several other payloads now used in clinical ADCs have been initially evaluated for chemotherapy as free drug, for example calicheamicin, PBD dimers and eribulin. but have failed because the extremely high potency of the cytotoxin (picomolar-low nanomolar IC₅₀ values) versus the typically low micromolar potency of standard chemotherapy drugs, such as paclitaxel and doxorubicin.

Currently, there is one marketed ADC and five ADCs in various clinical stages all based on payload DXd, a synthetic derivative of exatecan (structures in FIG. 6) and based on linker-drug deruxtecan (FIG. 8). DXd and exatecan are both family member of the camptothecins (topoisomerase 1 inhibitors). In the past, exatecan has been clinically evaluated as a stand-alone chemotherapy drug (exatecan mesylate, DX-89510, as preclinical studies indicated it was more potent than SN-38-based irinotecan (CPT-11) against various tumour xenograft models, including CPT-11-resistant tumours. In addition, exatecan was found not be a substrate for the Pgp transporter that confers multidrug resistance, whereas SN-38 is a weak substrate for Pgp. However, in clinical studies only a modest effect was observed for exatecan mesylate on various cancer types, summarized in Venditto and Simanek, Mol. Pharmaceut. 2010, 7, 307-349, incorporated by reference, with the most common drug-related toxicity being neutropenia. A phase III study comparing exatecan and gemcitabine showed no extended survival time versus gemcitabine therapy alone. As a consequence, development of exatecan mesylate as free drug was discontinued.

Subsequent exatecan-based studies focused on the development of DE-310, a macromolecular carrier system where DX-8951f is covalently linked to carboxymethyl dextran polyalcohol (CM-Dex-PA) via a Gly-Gly-Phe-Gly (GGFG) tetrapeptide spacer. The GGFG peptide spacer of DE-310 was designed to be cleaved by specific cysteine proteases that are upregulated in the tumour microenvironment and indeed preclinical studies and indeed it had been demonstrated that a single dose of 11.4 mg/kg of DE-310 exhibited stronger anti-tumour activity in mice than repeated doses (10 mg/kg daily for 5 d) of DX-8951f alone. However, the benefit of slow release, potentially due to the enhanced permeation retention (EPR), was not confirmed a phase 1 study for treatment of human cancers, as reported by Wente et al., Invest New Drugs. 2005, 23, 339, incorporated by reference.

Most recently, exatecan was also considered as a payload for antibody-drug conjugates and various linker formats were screened, as for example reported by Nakada et al., Bioorg. Med. Chem. Lett. 2016, 26, 1542-1545, incorporated by reference. However, it was observed that exatecan-based ADCs showed extensive aggregation, up to 26%. In addition, it was found that direct attachment of GGFG linker to the exatecan amino group led to incomplete proteolytic removal of the GGFG peptide spacer, releasing a mixture of both free DX-8951 as well as G-DX-8951, i.e. N-glycyl-exatecan (see FIG. 6). In fact, the earlier clinical study of DE-310 has demonstrated that G-DX-8951 concentration in tumour tissues is approximately 10-fold greater than DX-8951 itself, suggesting that G-DX-8951 might partially contribute to the cytotoxic activity of DE-310, as reported by Wente et al., Invest New Drugs. 2005, 23, 339, incorporated by reference. At the same time, preclinical data had indicated that while the inhibitory activity of DX-8951 and G-DX-8951 on topoisomerase-I activity is comparable, the in vitro cytotoxicity of DX-8951 is about 20-190 times more potent than G-DX-8951, as reported by Shiose et al., Biol. Pharm. Bull. 2007, 30, 2365-2370, incorporated by reference. Finally, various N-aminoacylated derivatives were found to display significantly reduced membrane permeability than exatecan/DX-8951 itself, as determined by PAMPA assay.

As a consequence of the reduced potency of G-DX-8951 versus DX-8951, the poor cell membrane permeability of aminoacylated exatecan derivatives in general and the significant aggregation potential of exatecan-based ADCs, a new exatecan-based linker technology was developed by Daiichi-Sankyo, called deruxtecan (depicted in FIG. 8) based on a N-hydroxyacylated DX-8951 derivative (DXd) in combination with a GGFG-aminal based linker. The linker-payload is conjugated with an antibody via cysteine residues after the interchain disulfide bounds are reduced with a reducing agent, thereby generating ADCs with DAR4 or DAR8 (ADC4a or ADC4b, respectively in FIG. 10A), depending on the stoichiometry of the reducing agent. Alternatively, a cysteine-engineered antibody can be used to generate an ADC with DAR2 (ADC5 in FIG. 10B). As the tetrapeptide is decomposed by lysosomal enzymes such as cathepsins B and L, which are highly expressed in tumour cells, it is supposed that DXd is released next via self-immolation of the liberated aminal part (with release of formaldehyde). As expected, DXd was found to be highly cell-permeable and as a consequence showed significant bystander effect, as reported by Ogitani et al. Cancer Sci. 2016, 107, 1039-1046, incorporated by reference. Therefore, ADCs based on deruxtecan may be beneficial in treating tumors with target heterogeneity. In addition, it was found that ADCs derived by cysteine conjugation with deruxtecan showed high stability, enabling high drug loading up to DAR8, as has been applied in three ADC programs, i.e. DS-8201a (ADC targeting HER2, currently marketed as Enhertu), U3-1402a (ADC targeting HER3) and DS-6157a (ADC targeting GRP20). In addition, two deruxtecan-based DAR4 ADCs are in various stages of clinical development, i.e. DS-1062a (ADC targeting TROP-2) and DS-7300a (ADC targeting B7-H3).

Besides the deruxtecan-based programs, three other ADCs with camptothecin payload have reached the clinic, two of which are based on SN-38, i.e. sacetizumab govetican (targeting TROP-2) and labetuzumab govetican (targeting CEACAM5), both based on SN-38. In fact, the first report by Walker et al., Bioorg. Med. Chem. Lett. 2002, 12, 217-219, incorporated by reference, of an ADC based on camptothecin payload comprised SN-38 (conjugated to anti-BR96 targeting antibody). Sacetizumab govetican, described by Sharkey et al., Clin. Cancer Res. 2015, 21, 5131-5138, incorporated by reference, has been approved as Trodelvy® for treatment of triple-negative breast cancer. A third ADC with a camptothecin payload (belotecan) that recently entered the clinic is SKB264.

Some preclinical studies with other variants of camptothecin-type payloads in the context of ADCs have recently also been disclosed. For example, Burke et al., Bioconj. Chem. 2009, 20, 1242-1250, incorporated by reference, described the preparation of antibody-drug conjugates (ADCs) with novel camptothecin analogues that are 10-1000 times more potent than camptothecin itself. ADCs bearing the potent camptothecin analogue, 7-butyl-9-amino-10,11-methylenedioxy-camptothecin, were highly potent and immunologically specific on a panel of cancer cell lines in vitro, and efficacious at well-tolerated doses in a renal cell carcinoma xenograft model. However, these ADCs were not further developed. Finally, a report was published in 2019 by Li et al., ACS Med. Chem. Lett. 2019, 10, 1386-1392 on ADCs with camptothecins inspired by exatecan but lacking the F-ring of the payload (for camptothecin ring numbering, see FIG. 6). It was reasoned that the chiral center in the F-ring complicates its synthesis and derivatization, so new camptothecins were synthesized that lack an F-ring in their payloads yet behave similarly to DXd-bearing conjugates in vitro and in vivo. In particular, ADCs having different degrees of bystander killing with the ability to release hydroxyl or thiol-bearing metabolites following peptide linker cleavage were benchmarked against deruxtecan-based ADCs and found to have at least the same potency against target-positive cells and enhanced potency against target-negative cells in co-culture (bystander killing). However, no ADCs based on exatecan itself were screened.

A wide variety of camptothecin-based ADCs have been described of which multiple are under clinical evaluation or have reached market-approval, all based on DXd, SN-38 or belotecan. However, to date no ADCs have been reported based on a linker format designed to liberate exatecan as active catabolite, potentially due to the high aggregation propensity reported by Nakada et al., Bioorg. Med. Chem. Lett. 2016, 26, 1542-1545, incorporated by reference. As a solution, DXd was developed, with the significant bystander effect of DXd contributing substantially to the value of the deruxtecan linker technology.

### SUMMARY OF THE INVENTION

The present inventors have surprisingly found that linkers with a cleavable peptide-PABC system are highly suitable for metal-free click or thiol conjugation of exatecan to antibodies, resulting in antibody-exatecan conjugates that show no or negligible aggregation propensity. The resulting ADCs were found to display significant in vivo efficacy. As such, the inventors have for the first time been able to prepare an ADC with exatecan payloads that exhibits efficacy in the same order of magnitude as the most efficacious antibody-camptothecin conjugates known to date, while showing no aggregation at all.

The invention first and foremost concerns an antibody-drug conjugate, having structure (1)

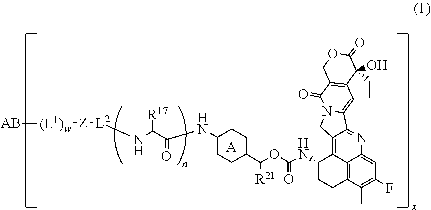

wherein:
AB is an antibody;
L¹ and L² are linkers;
w is 0 or 1;
Z is a connecting group obtained by a metal-free click reaction or by thiol ligation;
each R¹⁷ is individually an amino acid side chain;
n is an integer in the range of 1-5;
A is a 5- or 6-membered aromatic or heteroaromatic ring;
x is an integer in the range of 1-8;
R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups.

The invention further concerns a process for the synthesis of the antibody-drug conjugate according to the invention, a linker-drug construct which is suitable to be used in the process according to the invention, the medical use of the antibody-drug conjugate according to the invention and a pharmaceutical composition comprising the antibody-drug conjugate according to the invention.

### DESCRIPTION OF THE FIGURES

FIG. 1A shows cyclic alkynes suitable for metal-free click chemistry, and preferred embodiments for reactive moiety Q. The list is not comprehensive, for example alkynes can be further activated by fluorination, by substitution of the aromatic rings or by introduction of heteroatoms in the aromatic ring. FIG. 1B depicts a range of reagents suitable for reaction with cysteine side-chains. Reagents may be monoalkylation type (A) or may be a cross-linker (B) for reaction with two cysteine side-chains.

FIG. 2 shows a representative (but not comprehensive) set of functional groups (F) that can be introduced into an antibody by engineering, by chemical modification, or by enzymatic means, which upon metal-free click reaction with a complementary reactive group Q lead to connecting group Z. Functional group F may be artificially introduced (engineered) into an antibody at any position of choice. Some functional groups F (e.g. nitrile oxide, quinone), may besides strained alkynes also react with strained alkenes, which as an example is depicted for triazine or tetrazine (bottom line). The pyridine or pyridazine connecting group is the product of the rearrangement of the tetrazabicyclo[2.2.2]octane connecting group, formed upon reaction of triazine or tetrazine with alkyne (but not alkene), respectively, with loss of N₂. Connecting groups Z are preferred connecting groups to be used in the present invention.

FIG. 3 shows the general scheme for preparation of antibody-drug conjugates by reaction of a monoclonal antibody (in most cases a symmetrical dimer) containing an x number of functionalities F. By incubation of antibody-(F)ₓ with excess of a linker-drug construct (Q-spacer-linker-payload) a conjugate is obtained by reaction of F with Q, forming bond Z.

FIG. 4 shows the general process for non-genetic conversion of a monoclonal antibody into an antibody containing probes for click conjugation or thiol ligation (F). The click probe may be on various positions in the antibody, depending on the technology employed. For example, the antibody may be converted into an antibody containing two click probes (structure on the left) or four click probes (bottom structure) or eight probes (structure on the right) for click conjugation.

FIG. 5A depicts a specific example of site-specific conjugation of a payload based on glycan remodeling of a full-length IgG followed by azide-cyclooctyne click chemistry. The IgG is first enzymatically remodeled by endoglycosidase-mediated trimming of all different glycoforms, followed by glycosyltransferase-mediated transfer of azido-sugar onto the core GlcNAc liberated by endoglycosidase. In the next step, the azido-remodeled IgG is subjected to an immune cell-engaging polypeptide, which has been modified with a single cyclooctyne for metal-free click chemistry (SPAAC), leading to a bispecific antibody of 2:2 molecular format. It is also depicted that the cyclooctyne-polypeptide construct will have a specific spacer between cyclooctyne and polypeptide, which enables tailoring of IgG-polypeptide distance or impart other properties onto the resulting bispecific antibody.

FIG. 5B depicts a specific example of site-specific conjugation of a payload based on glycan remodeling of a full-length IgG followed by thiol alkylation chemistry. The IgG is first enzymatically remodeled by endoglycosidase-mediated trimming of all different glycoforms, followed by glycosyltransferase-mediated transfer of a thiol-modified (and disulfide-protected) sugar derivative onto the core GlcNAc liberated by endoglycosidase. In the next step, the remodeled IgG is subjected to reduction (to convert the disulfide into thiol), potentially followed by oxidation, then reaction with a payload modified with a suitable thiol-reactive reagent.

FIG. 6 shows the structures of topoisomerase inhibitors DXd and exatecan, as well as N-glycyl-exatecan (G-DX-8951). Ring numbering is indicated for exatecan.

FIG. 7 depicts the structures of BCN-linker-drugs 1-3, suitable for application in ADCs by conjugation to azido-modified antibodies. Structures 1-3 contain a peptide-PABC cleavable linker, wherein the peptide exists of Val-Ala (1a and 2a) or Val-Cit (1b or 2b). Structures 1 and 2 are designed for direct release of payload, for structure 3 an additional moiety is included (N,N′-dimethylethylenediaminocarbonyl) for release of payload through cyclization. Structures 1 and 2 contain cytotoxic payload exatecan, structure 3 contains cytotoxic payload SN-38.

FIG. 8 depicts the structure of DXd-based maleimide-linker-drug 4 (also known as deruxtecan), suitable for application in ADCs by conjugation to free cysteine side-chains.

FIG. 9 shows the structures of ADCs obtained by enzymatic remodeling of N-glycan (for introduction of azido-sugar) followed by metal-free click conjugation with linker-drugs 1 or 2. Remodeling and conjugation with 1 or 2 at the native N297 glycan only, provides ADC1a or ADC2, respectively, both DAR4. Remodeling and conjugation with 1 at the native N297 glycan plus an additionally engineered N-glycosylation site (e.g. HC-L²⁰¹N), provides ADC1b, DAR8.

FIG. 10A shows the structure of an ADC obtained by reduction of native disulfide bonds, followed by conjugation with deruxtecan 4, providing ADC4a (average DAR4) or ADC4b (average DAR8), depending on specific conditions employed (stoichiometry of reducing agent, e.g. TCEP or DTT, and deruxtecan).

FIG. 10B shows the structure of an ADC obtained from an antibody with an engineered cysteine, which may be a mutant of a natural amino acid, a cysteine insertion or a cysteine in a peptide fused to the N- or C-terminus of the antibody, followed by conjugation with deruxtecan 4, providing ADC5 (average DAR2).

FIG. 11 shows a range of antibody variants, with azide group or thiol group available, as starting materials for subsequent conversion to antibody conjugates.

FIG. 12 depicts the HIC profile of azido-trastuzumab (after enzymatic remodeling), ADC1a (DAR4) and ADC4a (DAR4). Azido-trastuzumab shows a retention time of 9.2 min, while ADC1a according to the invention shows a single peak with retention time 9.8 min, so a relative retention time of 1.06. In contrast, control ADC ADC4a shows a multitude of peaks eluting from the HIC column (10.2 min until>12.3 min), with relative retention times versus azido-trastuzumab of 1.11 until>1.33.

FIG. 13 shows the RP-HPLC trace for the conjugation of trastuzumab-(6-azidoGalNAc)₂ with 25a.

FIG. 14 shows the RP-UPLC traces under reducing conditions for the conjugation of trastuzumab S239C mutant trast-v3 with maleimide-exatecan variants 54a, 57b, 59a or 60a.

FIG. 15 shows the RP-UPLC trace under reducing conditions for the conjugation of trastuzumab GalProSH trast-v2 with maleimide-exatecan variants 57b or 60a.

FIG. 16 shows the RP-UPLC trace under reducing conditions for the conjugation of trastuzumab trast-v4 with maleimide-exatecan variants 60a or 57b according to general procedure A.

FIG. 17 shows the RP-UPLC trace under reducing conditions for the conjugation of rituximab rit-v4 with maleimide-exatecan variants 60a or 57b according to general procedure A.

FIG. 18 shows the RP-UPLC trace under reducing conditions for the conjugation of trastuzumab trast-v4 with maleimide-exatecan variants 60a or 57b according to general procedure B.

FIG. 19 shows the results of an in vivo efficacy study monitoring of tumour volume in mice engrafted with BT-474 cell line and administration of vehicle, Kadcyla (T-DM1) and DAR4 ADCs based on exatecan (ADC1a) and SN-38 (ADC3), all single dose. ADC1a according to the invention shows complete tumour disappearance at a dose of 12 mg/kg, which is significantly improved in comparison to control ADC3.

FIG. 20A shows the results of an in vivo efficacy study monitoring of tumour volume in mice engrafted with BT-474 cell line and administration of vehicle, and DAR4 ADCs based on exatecan (ADC1a) and DXd (ADC4a), all single dose. FIG. 20B is a zoom of FIG. 20A. ADC1a according to the invention and control ADC4a do not exhibit a significant difference in efficacy, with a partial response at 4 mg/kg and complete tumour regression for both ADCs at a dose of 12 mg/kg.

FIG. 21 shows the results of an in vivo efficacy study monitoring of tumour volume in mice engrafted with BT-474 cell line and administration of vehicle, and DAR4 plus DAR8 ADCs based on exatecan (ADC1a (DAR4) and ADC1b (DAR8)) and DXd (ADC4a (DAR4) and ADC4b (DAR4)), all single dose.

FIG. 22 shows the results of an in vivo efficacy study monitoring of tumour volume in mice engrafted with BT-474 cell line and administration of vehicle, and DAR4 ADCs based on exatecan with short spacer (ADC1a (DAR4)) or long spacer (ADC2 (DAR4)). ADC1a and ADC2 according to the invention do not exhibit a significant difference in efficacy.

### DETAILED DESCRIPTION OF THE INVENTION

### Definitions

The verb “to comprise”, and its conjugations, as used in this description and in the claims is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

The compounds disclosed in this description and in the claims may comprise one or more asymmetric centres, and different diastereomers and/or enantiomers may exist of the compounds. The description of any compound in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer.

The compounds disclosed in this description and in the claims may further exist as R and S stereoisomers. Unless stated otherwise, the description of any compound in the description and in the claims is meant to include both the individual R and the individual S stereoisomers of a compound, as well as mixtures thereof. When the structure of a compound is depicted as a specific S or R stereoisomer, it is to be understood that the invention of the present application is not limited to that specific S or R stereoisomer.

The compounds disclosed in this description and in the claims may further exist as R and S stereoisomers. Unless stated otherwise, the description of any compound in the description and in the claims is meant to include both the individual R and the individual S stereoisomers of a compound, as well as mixtures thereof. When the structure of a compound is depicted as a specific S or R stereoisomer, it is to be understood that the invention of the present application is not limited to that specific S or R stereoisomer.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the invention of the present application is not limited to that specific endo or exo diastereomer.

The compounds according to the invention may exist in salt form, which are also covered by the present invention. The salt is typically a pharmaceutically acceptable salt, containing a pharmaceutically acceptable anion. The term “salt thereof” means a compound formed when an acidic proton, typically a proton of an acid, is replaced by a cation, such as a metal cation or an organic cation and the like. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically acceptable” salt means a salt that is acceptable for administration to a patient, such as a mammal (salts with counter ions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The term “protein” is herein used in its normal scientific meaning. Herein, polypeptides comprising about 10 or more amino acids are considered proteins. A protein may comprise natural, but also unnatural amino acids.

The term “antibody” is herein used in its normal scientific meaning. An antibody is a protein generated by the immune system that is capable of recognizing and binding to a specific antigen.

An antibody is an example of a glycoprotein. The term antibody herein is used in its broadest sense and specifically includes monoclonal antibodies, polyclonal antibodies, dimers, multimers, multi-specific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole immunoglobulins, but also antigen-binding fragments of an antibody. Furthermore, the term includes genetically engineered antibodies and derivatives of an antibody. Antibodies, fragments of antibodies and genetically engineered antibodies may be obtained by methods that are known in the art.

An “antibody fragment” is herein defined as a portion of an intact antibody, comprising the antigen-binding or variable region thereof. Examples of antibody fragments include Fab, Fab′, F(ab′)₂, and Fv fragments, diabodies, minibodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen” is herein defined as an entity to which an antibody specifically binds.

The terms “specific binding” and “specifically binds” is herein defined as the highly selective manner in which an antibody or antibody binds with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “substantial” or “substantially” is herein defined as a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

A “linker” is herein defined as a moiety that connects two or more elements of a compound. For example in an antibody-conjugate, an antibody and a payload are covalently connected to each other via a linker. A linker may comprise one or more linkers and spacer-moieties that connect various moieties within the linker.

A “spacer” or spacer-moiety is herein defined as a moiety that spaces (i.e. provides distance between) and covalently links together two (or more) parts of a linker. The linker may be part of e.g. a linker-construct, the linker-conjugate or a bioconjugate, as defined below.

A “self-immolative group” is herein defined as a part of a linker in an antibody-drug conjugate with a function is to conditionally release free drug at the site targeted by the ligand unit. The activatable self-immolative moiety comprises an activatable group (AG) and a self-immolative spacer unit. Upon activation of the activatable group, for example by enzymatic conversion of an amide group to an amino group or by reduction of a disulfide to a free thiol group, a self-immolative reaction sequence is initiated that leads to release of free drug by one or more of various mechanisms, which may involve (temporary) 1,6-elimination of a p-aminobenzyl group to a p-quinone methide, optionally with release of carbon dioxide and/or followed by a second cyclization release mechanism. The self-immolative assembly unit can part of the chemical spacer connecting the antibody and the payload (via the functional group). Alternatively, the self-immolative group is not an inherent part of the chemical spacer, but branches off from the chemical spacer connecting the antibody and the payload.

A “conjugate” is herein defined as a compound wherein an antibody is covalently connected to a payload via a linker. A conjugate comprises one or more antibodies and/or one or more payloads.

The term “payload” refers to the moiety that is covalently attached to a targeting moiety such as an antibody, but also to the molecule that is released from the conjugate upon uptake of the protein conjugate and/or cleavage of the linker. Payload thus refers to the monovalent moiety having one open end which is covalently attached to the targeting moiety via a linker and also to the molecule that is released therefrom. In the context of the present invention, the payload is exatecan.

### The Invention

The inventors have developed an antibody-drug conjugate, containing exatecan as cytotoxic payload and a cleavable peptide-PABC system, which show no or negligible aggregation propensity. The resulting ADCs were found to display significant in vivo efficacy.

The invention first and foremost concerns the antibody-drug conjugate. In a second aspect, the invention concerns a process for the synthesis of the antibody-drug conjugate according to the invention. In a third aspect, the invention concerns a linker-drug construct which is suitable to be used in the process according to the invention. In a fourth aspect, the invention concerns the medical use of the antibody-drug conjugate according to the invention, as well as a pharmaceutical composition comprising the antibody-drug conjugate according to the invention. The skilled person understands that all aspects are linked, and that everything said for the antibody-drug conjugate according to the invention equally applies to the process according to the invention, the linker-drug construct according to the invention, the uses according to the invention and the composition according to the invention, and vice versa.

The invention can be defined according to the following list of preferred embodiments:
1. An antibody-drug conjugate, having structure (1)

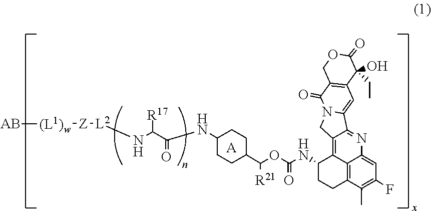

wherein:
   AB is an antibody;
   L¹ and L² are linkers;
   w is 0 or 1;
   Z is a connecting group obtained by a metal-free click reaction or by thiol ligation;
   each R¹⁷ is individually an amino acid side chain;
   n is an integer in the range of 1-5;
   A is a 5- or 6-membered aromatic or heteroaromatic ring;
   x is an integer in the range of 1-8;
   R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups.
2. The antibody-drug conjugate according to embodiment 1, wherein L² has structure (2)

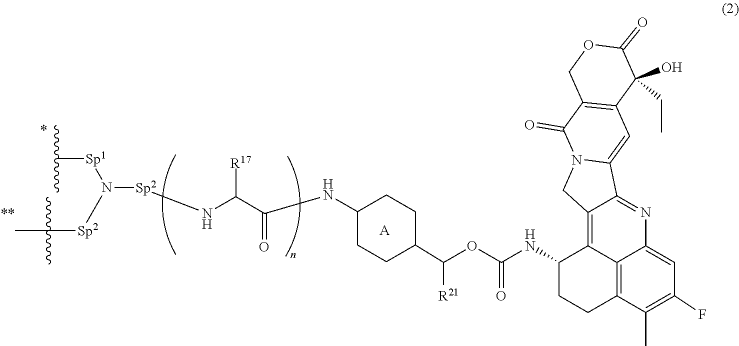

wherein
   the wavy bond labeled with * is connected to Z and the wavy bond labeled with ** is connected to NH;
   Sp¹ and Sp² are each individually spacer moieties;
   n, A, R¹⁷ and R²¹ are as defined as in embodiment 1.
3. The antibody-drug conjugate according to embodiment 2, wherein each occurrence of Sp² is the same, each occurrence of (NH—CR¹⁷—CO)ₙ is the same, each occurrence of A is the same and each occurrence of R²¹ is the same.
4. The antibody-drug conjugate according to any one of the preceding embodiments, wherein L², preferably Sp¹, comprises a comprises a sulfamide group according to structure (3)

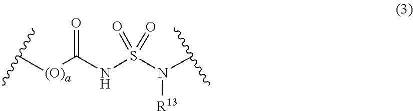

wherein
   a=0 or 1, and
   R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is a second occurrence of C(O)X connected to N via a spacer moiety.
5. The antibody-drug conjugate according to embodiment 4, wherein L², preferably Sp¹, comprises two groups of formula (3).
6. The antibody-drug conjugate according to any one of the preceding embodiments, wherein each occurrence of n is 2.
7. The antibody-drug conjugate according to any one of the preceding embodiments, wherein each occurrence of (NH—CR¹⁷—CO)ₙ is selected from Val-Cit, Val-Ala, Val-Lys, Val-Arg, AcLys-Val-Cit, AcLys-Val-Ala, Phe-Cit, Phe-Ala, Phe-Lys, Phe-Arg, Ala-Lys, Leu-Cit, Ile-Cit, Trp-Cit, Ala-Ala-Asn, Ala-Asn and Lys, preferably from Val-Cit, Val-Ala, Val-Lys, Phe-Cit, Phe-Ala, Phe-Lys, Ala-Ala-Asn, most preferably Val-Cit or Val-Ala.
8. The antibody-drug conjugate according to any one of the preceding embodiments, wherein Z has a structure selected from (Z1)-(Z8) and (Z11)-(Z23):

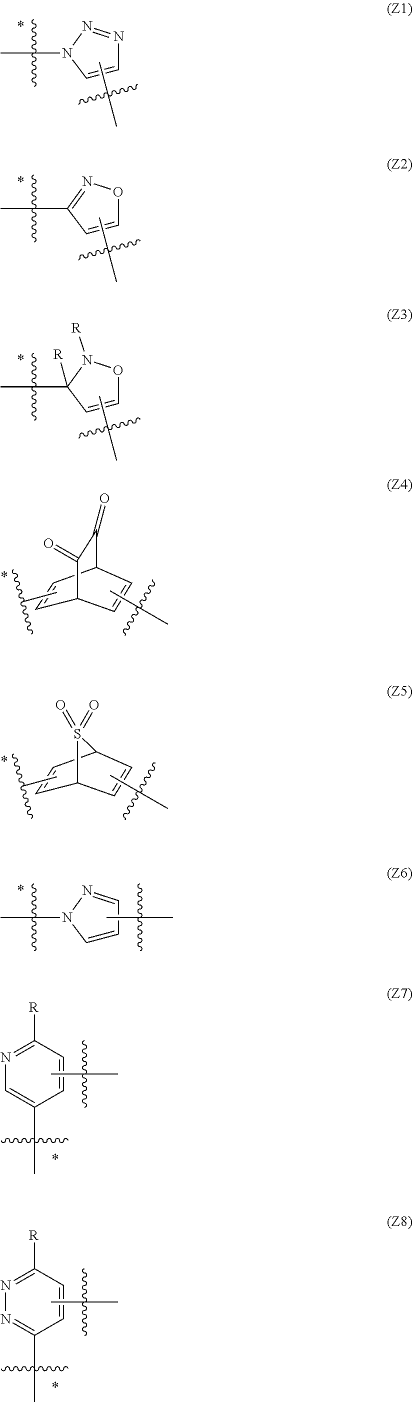

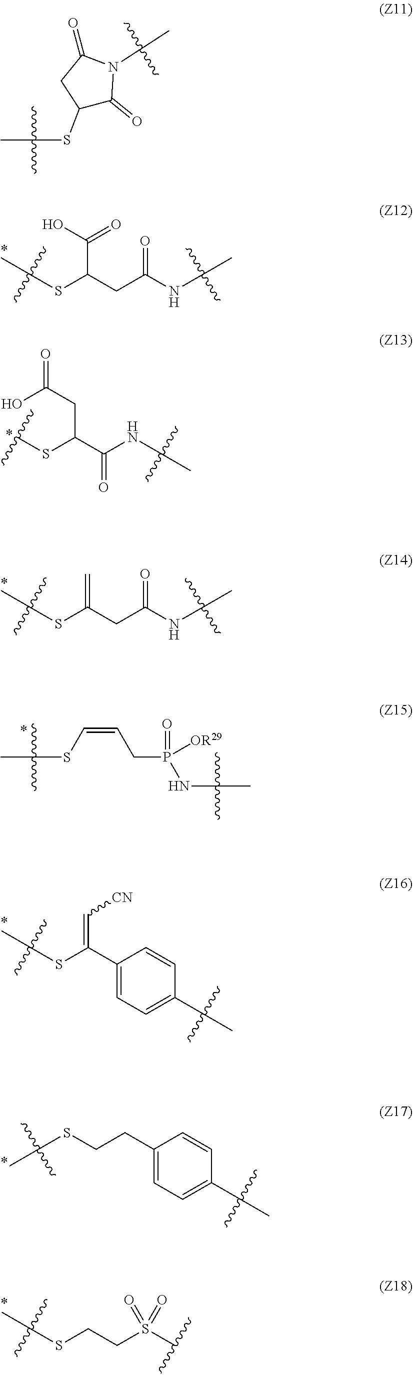

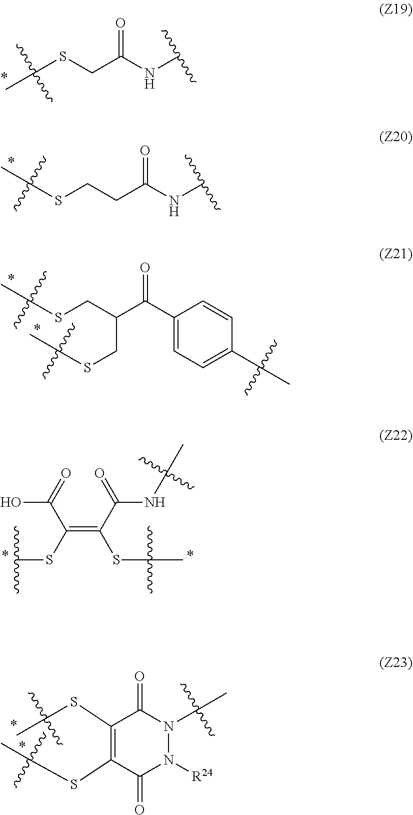

wherein:
   the wavy bond labelled with an * is connected to AB, optionally via L¹, and the other wavy bond to L²,
   functional groups R in (Z3), (Z7) and (Z8) is selected from hydrogen, C₁-C₂₄ alkyl groups, C₂-C₂₄ acyl groups, C₃-C₂₄ cycloalkyl groups, C₂-C₂₄ (hetero)aryl groups, C₃-C₂₄ alkyl(hetero)aryl groups, C₃-C₂₄ (hetero)arylalkyl groups and C₁-C₂₄ sulfonyl groups, each of which may optionally be substituted and optionally be interrupted by one or more heteroatoms selected from O, S and NR³² wherein R³² is independently selected from the group consisting of hydrogen and C₁-C₄ alkyl groups;
   R²⁴ is H or C₁₋₁₂ alkyl, preferably H or C₁₋₆ alkyl;
   R²⁹ is C₁₋₁₂ alkyl, preferably C₁₋₄ alkyl, most preferably ethyl.
9. The antibody-drug conjugate according to any one of the preceding embodiments, which has structure (1a):

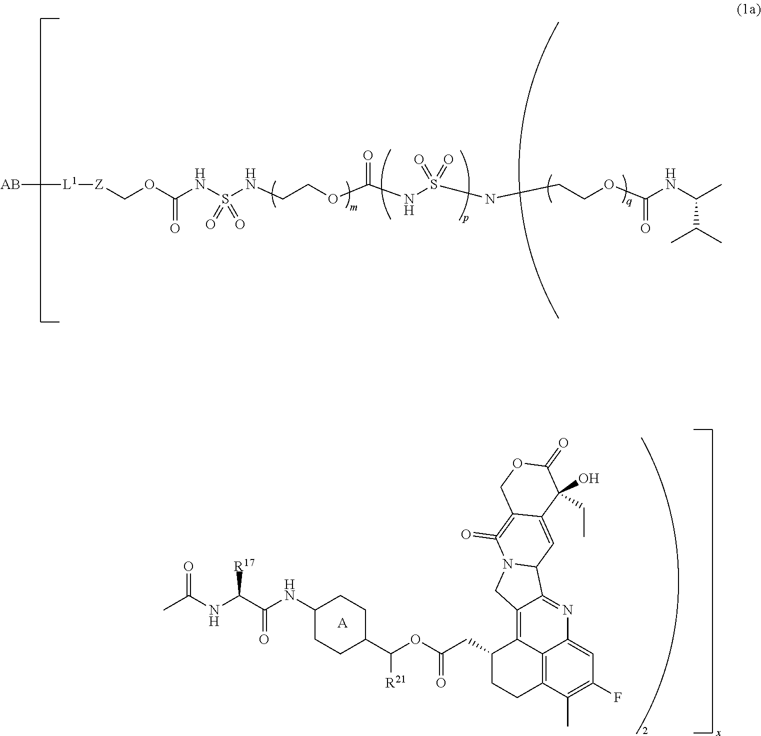

wherein:
   AB, L¹, w, Z, A, R²¹ and x are as defined in embodiment 1;
   R¹⁷ is CH₃ or CH₂CH₂CH₂NHC(O)NH₂;
   m is an integer in the range of 1-10;
   q is an integer in the range of 0-10;
   p is 0 or 1.
10. The antibody-drug conjugate according to any one of the preceding embodiments, wherein w=0 and the conjugation site is a cysteine residue, preferably wherein the cysteine residue is naturally present in the antibody, optionally after reduction of a disulfide bond, or wherein the cysteine residue is engineered, preferably by substitution of an amino acid by cysteine, cysteine insertion or introduction of a single cysteine residue or a peptide fragment containing a cysteine residue at the N- or C-terminus.
11. The antibody-drug conjugate according to any one of embodiments 1-9, which has structure (1b):

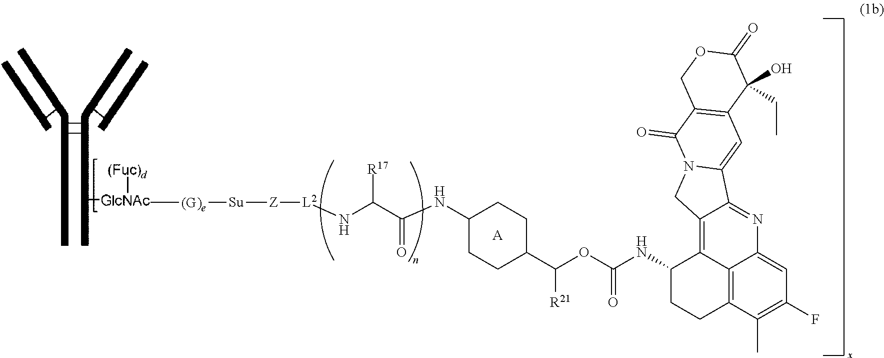

wherein:
   Z, L², R¹⁷, A, R²¹, n and x are as defined in embodiment 1;
   e is an integer in the range of 0-20;
   Su is a monosaccharide;
   G is a monosaccharide moiety;
   GlcNAc is an N-acetylglucosamine moiety;
   Fuc is a fucose moiety;
   d is 0 or 1.
12. The antibody-drug conjugate according to any one of the preceding embodiments, wherein antibody AB targets HER2.
13. Process for the synthesis of the antibody-drug conjugate according to any one of embodiments 1-12, comprising reacting
   (i) a modified antibody of structure AB-((L¹)_{w}-F)ₓ, wherein:
      AB is an antibody;
      L¹ is a linker;
      w is 0 or 1;
      F is a click probe capable of reacting with Q in a metal-free click reaction or a thiol or precursor thereof;
      x is an integer in the range of 1-8;
   with
   (ii) a linker-drug construct according to structure (5):

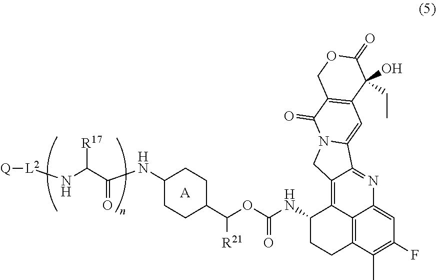

wherein:
   L² is a linker;
   Q is a click probe capable of performing a metal-free click reaction or a thiol-reactive probe;
   each R¹⁷ is individually an amino acid side chain;
   n is an integer in the range of 1-5;
   A is a 5- or 6-membered aromatic or heteroaromatic ring;
   x is an integer in the range of 1-8;
   R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups, to form an antibody-drug conjugate wherein the drug is covalently attached to the antibody via connecting group Z that is formed by a metal-free click reaction between Q and F or by thiol ligation between Q and F.
14. The process according to embodiment 13, wherein the click probe Q comprises a cyclic alkyne moiety or a cyclic alkene moiety, or wherein the thiol-reactive probe Q comprises a maleimide moiety.
15. The process according to embodiment 14, wherein the click probe Q is selected from the group consisting of (Q22)-(Q36):

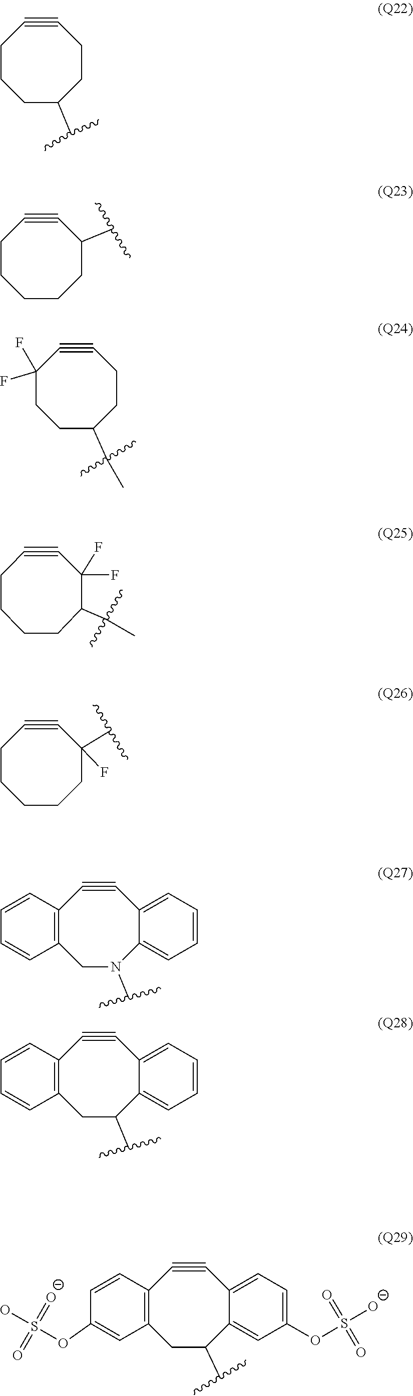

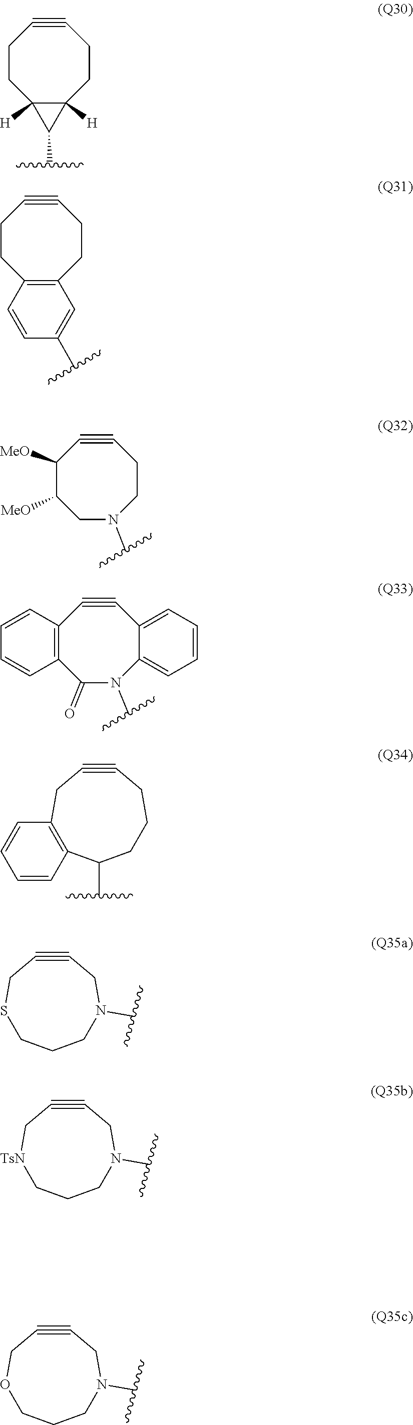

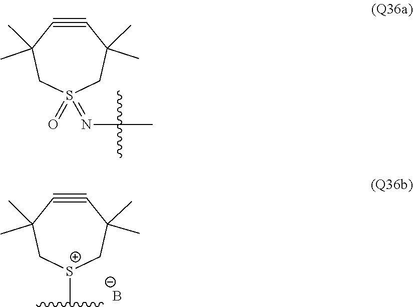

wherein B⁽⁻⁾ is an anion;
or wherein the (hetero)cycloalkynyl moiety Q is according to structure (Q37):

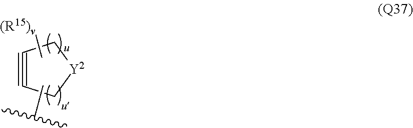

wherein:
   R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
   Y² is C(R³¹)₂, O, S or NR³¹, wherein each R³¹ individually is R¹⁵ or a second occurrence of the exatecan payload connected via a spacer moiety;
   u is 0, 1, 2, 3, 4 or 5;
   u′ is 0, 1, 2, 3, 4 or 5, wherein u+u′=4, 5, 6, 7 or 8;
   v=an integer in the range 8-16;
preferably wherein the cyclooctynyl moiety Q is according to structure (Q38):

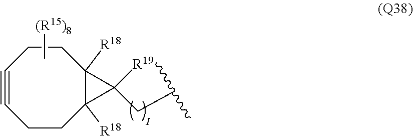

wherein
   R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
   R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
   R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of the exatecan payload connected via a spacer moiety; and
   I is an integer in the range 0 to 10;
or wherein the (hetero)cyclooctynyl moiety Q is according to structure (Q39):

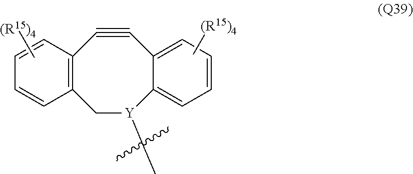

wherein
   R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
   Y is N or CR¹⁵;
or wherein the heterocycloheptynyl moiety Q is according to structure (Q36a):

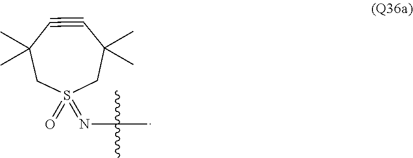

17. The process according to embodiment 14, wherein the click probe Q is selected from the group consisting of, optionally substituted, (hetero)cyclopropenyl group, (hetero)cyclobutenyl group, trans-(hetero)cycloheptenyl group, trans-(hetero)cyclooctenyl group, trans-(hetero)cyclononenyl group or trans-(hetero)cyclodecynyl group, preferably click probe Q is selected from the group consisting of (Q40)-(Q50):

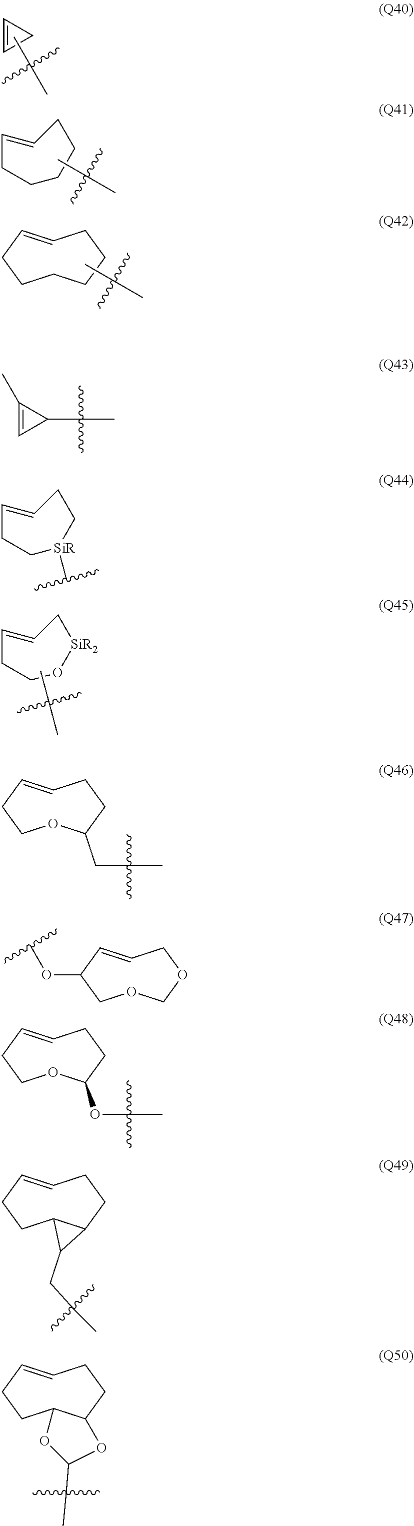

wherein the R group(s) on Si in (Q44) and (Q45) is alkyl or aryl.
18. The process according to embodiment 13 or 14, wherein the thiol-reactive probe Q is selected from the group consisting of (Q51)-(Q65):

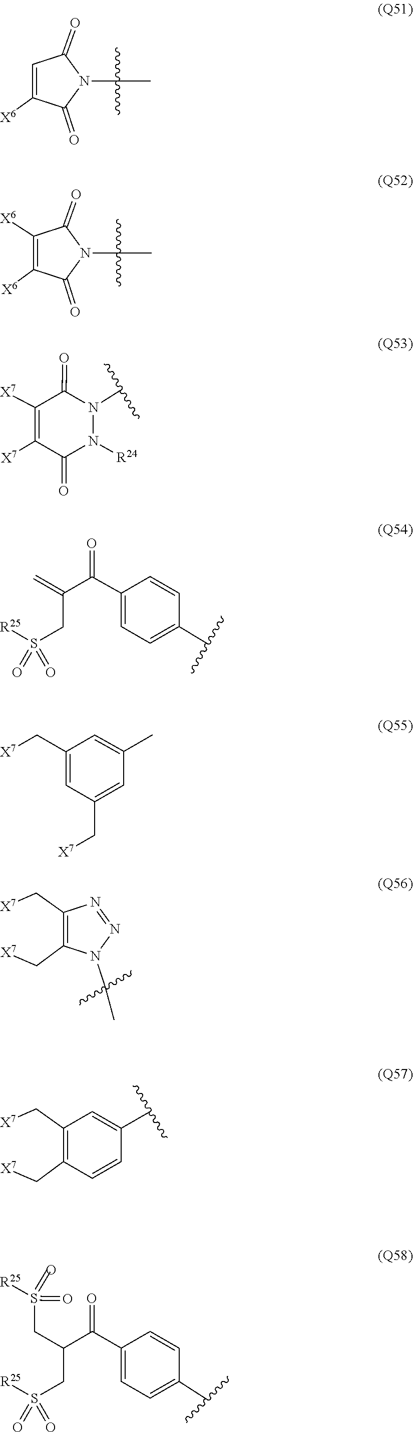

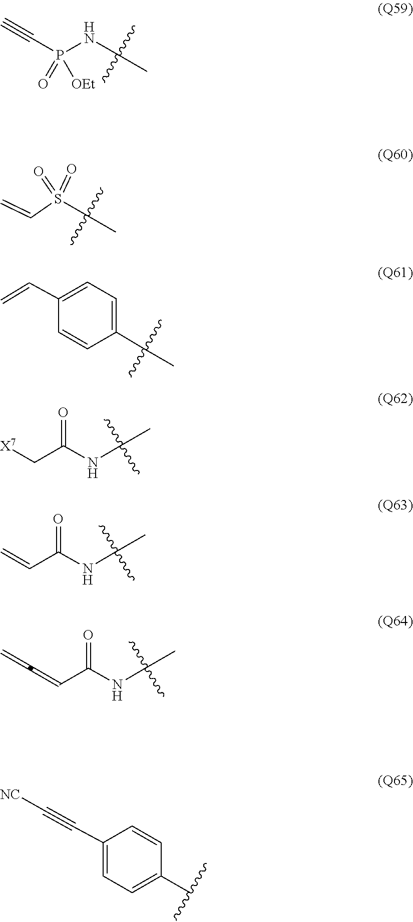

wherein:
   X⁶ is H, halogen, PhS, MeS;
   X⁷ is halogen, PhS, MeS;
   R²⁴ is H or C₁₋₁₂ alkyl, preferably H or C₁₋₆ alkyl;
   R²⁵ is H, C₁₋₁₂ alkyl, C₁₋₁₂ aryl, C₁₋₁₂ alkaryl or C₁₋₁₂ aralkyl, preferably H or para-methylphenyl;
   wherein the aromatic ring of (Q55) and (Q57) may optionally be a heteroaromatic ring, such as a phenyl or pyridine ring.
19. The process according to any one of embodiments 13-18, wherein click probe F is selected from the group consisting of azide, tetrazine, triazine, nitrone, nitrile oxide, nitrile imine, diazo compound, ortho-quinone, dioxothiophene and sydnone, preferably click probe F is an azide moiety.
20. The process according to any one of embodiments 13-19, wherein the click reaction is a 1,3-dipolar cycloaddition or a (4+2) cycloaddition.
21. The process according to any one of embodiments 13-18, wherein F is a thiol.
22. The process according to any one of embodiments 13-18 or 21, wherein the thiol ligation is a nucleophilic reaction, preferably a Michael addition or a nucleophilic substitution.
23. The process according to any one of embodiments 13-18 or 21-22, wherein w=0 and the conjugation site is a cysteine residue, preferably wherein the cysteine residue is naturally present in the antibody, optionally after reduction of a disulfide bond, or wherein the cysteine residue is engineered, preferably by substitution of an amino acid by cysteine, cysteine insertion or introduction of a single cysteine residue or a peptide fragment containing a cysteine residue at the N- or C-terminus.
24. The process according to any one of embodiments 13-23, wherein w=1 and AB-((L¹)_{w}-F)ₓ is according to structure (6):

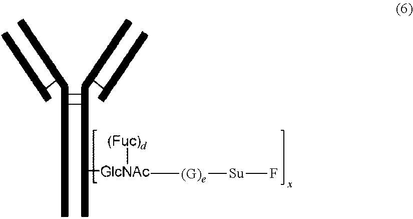

wherein:
   e is an integer in the range of 0-10;
   Su is a monosaccharide;
   G is a monosaccharide moiety;
   GlcNAc is an N-acetylglucosamine moiety;
   Fuc is a fucose moiety;
   d is 0 or 1.
25. A linker-drug construct according to structure (5):

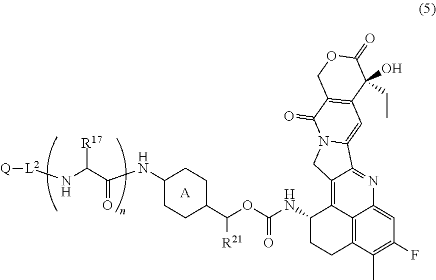

wherein:
   L² is a linker;
   Q is a click probe capable of performing a metal-free click reaction or a thiol-reactive probe;
   each R¹⁷ is individually an amino acid side chain;
   n is an integer in the range of 1-5;
   A is a 5- or 6-membered aromatic or heteroaromatic ring;
   x is an integer in the range of 1-8;
   R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³
   wherein R²³ is independently selected from the group consisting of hydrogen and C₁-C₄ alkyl groups.
26. The linker-drug construct according to embodiment 25, wherein the click probe Q comprises a cyclic alkyne moiety or a cyclic alkene moiety, or wherein the thiol-reactive probe Q comprises a maleimide moiety.
27. The linker-drug construct according to embodiment 25, wherein the click probe Q is selected from the group consisting of (Q22)-(Q36):

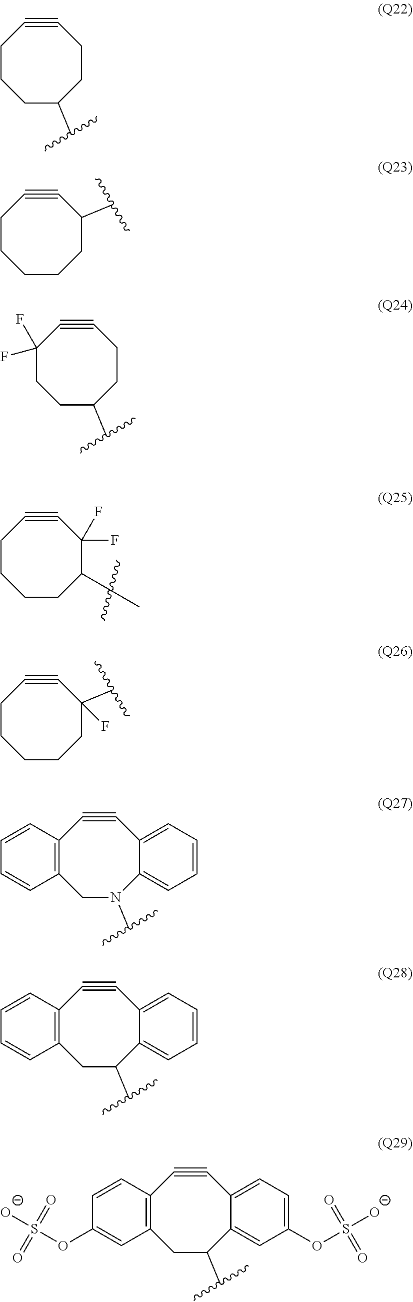

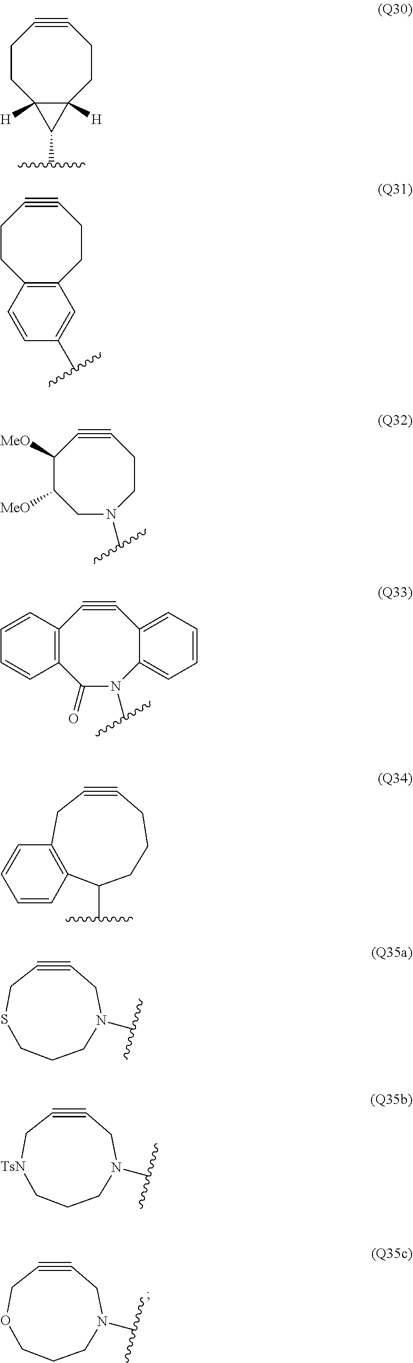

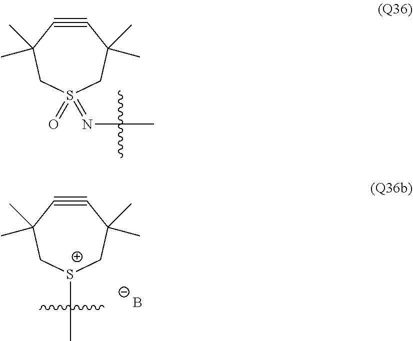

wherein B⁽⁻⁾ is an anion;
or wherein the (hetero)cycloalkynyl moiety Q is according to structure (Q37):

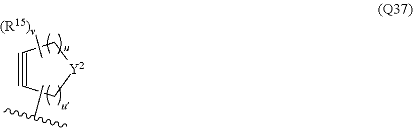

wherein:
   R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
   Y² is C(R³¹)₂, O, S or NR³¹, wherein each R³¹ individually is R¹⁵ or a second occurrence of the exatecan payload connected via a spacer moiety;
   u is 0, 1, 2, 3, 4 or 5;
   u′ is 0, 1, 2, 3, 4 or 5, wherein u+u′=4, 5, 6, 7 or 8;
   v=an integer in the range 8-16;
preferably wherein the cyclooctynyl moiety Q is according to structure (Q38):

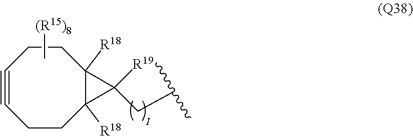

wherein
   R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
   R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
   R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of the exatecan payload connected via a spacer moiety; and
   I is an integer in the range 0 to 10;
or wherein the (hetero)cyclooctynyl moiety Q is according to structure (Q39):

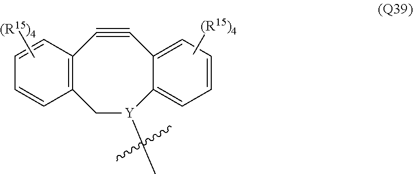

wherein
   R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
   Y is N or CR¹⁵;
or wherein the heterocycloheptynyl moiety Q is according to structure (Q36a):

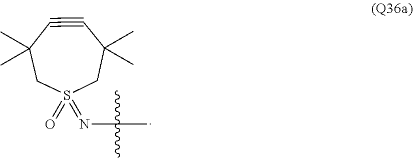

28. The linker-drug construct according to embodiment 26, wherein the click probe Q is selected from the group consisting of, optionally substituted, (hetero)cyclopropenyl group, (hetero)cyclobutenyl group, trans-(hetero)cycloheptenyl group, trans-(hetero)cyclooctenyl group, trans-(hetero)cyclononenyl group or trans-(hetero)cyclodecynyl group, preferably click probe Q is selected from the group consisting of (Q40)-(Q50):

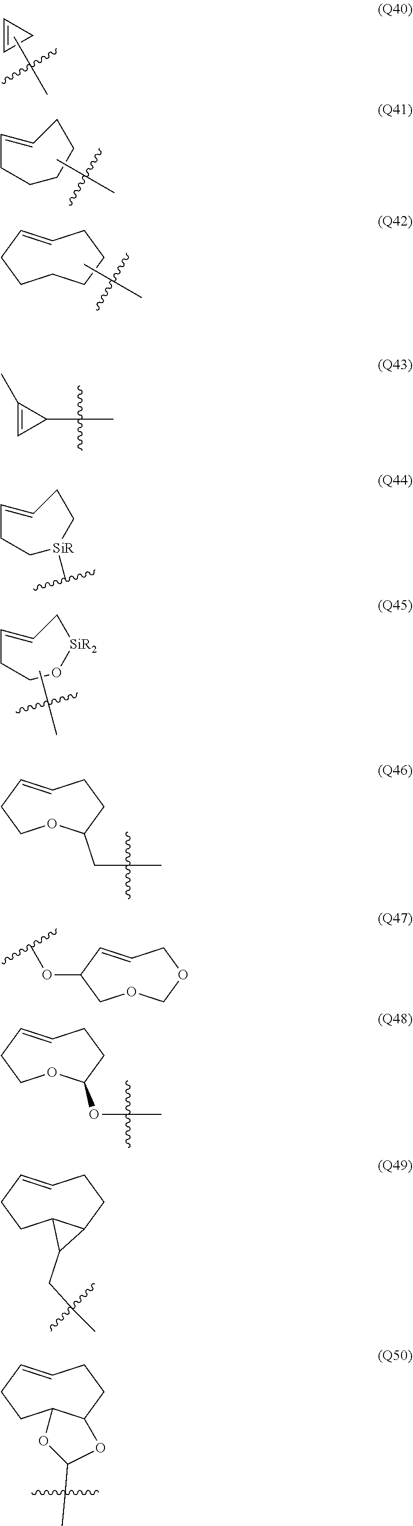

wherein the R group(s) on Si in (Q44) and (Q45) is alkyl or aryl.
29. The linker-drug construct according to embodiment 25 or 26, wherein thiol-reactive probe Q is selected from the group consisting of (Q51)-(Q65):

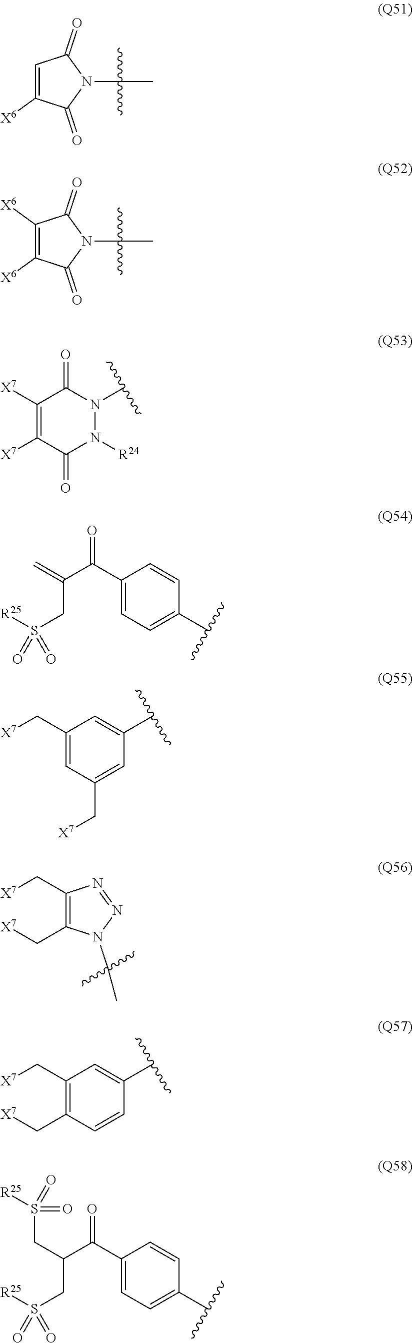

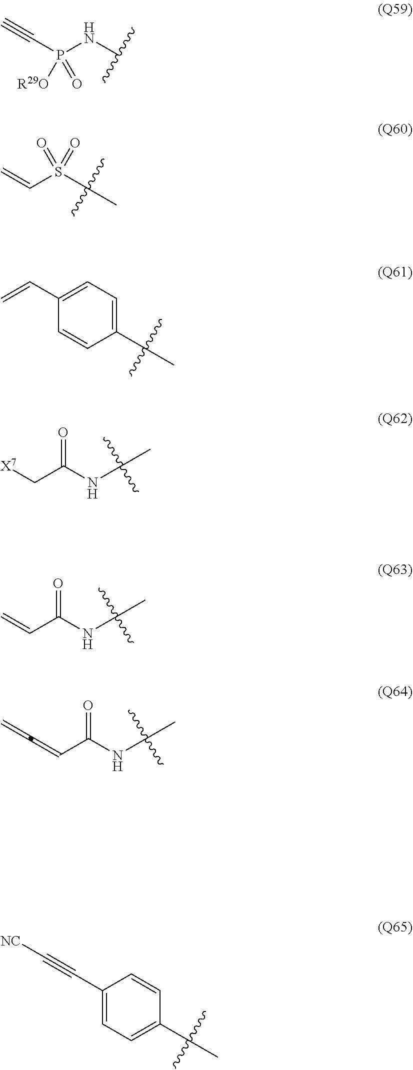

wherein:
   X⁶ is H, halogen, PhS, MeS;
   X⁷ is halogen, PhS, MeS;
   R²⁴ is H or C₁₋₁₂ alkyl, preferably H or C₁₋₆ alkyl;
   R²⁵ is H, C₁₋₁₂ alkyl, C₁₋₁₂ aryl, C₁₋₁₂ alkaryl or C₁₋₁₂ aralkyl, preferably H or para-methylphenyl;
   R²⁹ is C₁₋₁₂ alkyl, preferably C₁₋₄ alkyl, most preferably ethyl;
   wherein the aromatic ring of (Q55) and (Q57) may optionally be a heteroaromatic ring, such as a phenyl or pyridine ring.
30. Pharmaceutical composition comprising the antibody-drug conjugate according to any one of embodiments 1-12 and a pharmaceutically acceptable carrier.
31. The antibody-drug conjugate according to any one of embodiments 1-12, for use in the treatment of a subject in need thereof.
32. The antibody-drug conjugate according to any one of embodiments 1-12, for use in the treatment of cancer.
33. The antibody-drug conjugate for use according to embodiment 32, wherein the cancer is a HER2-positive cancer, preferably breast, stomach, colon, lung, pancreatic, urothelial, brain or ovarian cancer.

### The Antibody-Drug Conjugate

The antibody-drug conjugate according to the invention has structure (1):

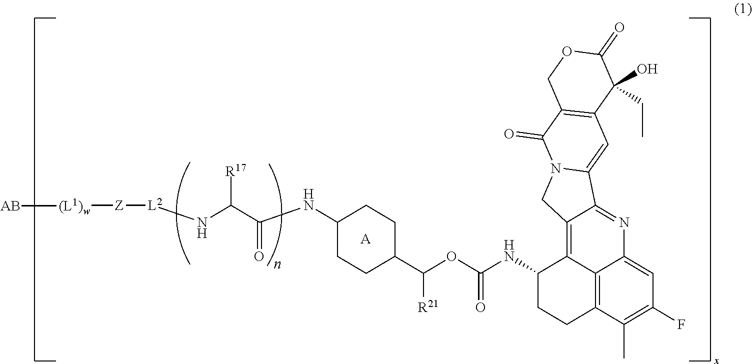

wherein:
   AB is an antibody;
   L¹ and L² are linkers;
   w is 0 or 1;
   Z is a connecting group obtained by a metal-free click reaction or by thiol ligation;
   each R¹⁷ is individually an amino acid side chain;
   n is an integer in the range of 1-5;
   A is a 6-membered aromatic or heteroaromatic ring;
   x is an integer in the range of 1-8;
   R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups.

The conjugate according to the invention contains a self-immolative group or cleavable linker, comprising a peptide spacer and a para-aminobenzyloxycarbonyl (PABC) moiety or derivative thereof.

The peptide spacer is defined by (NH—CR¹⁷—CO)ₙ, wherein R¹⁷ represents an amino acid side chain as known in the art. Herein, the amino acid may be a natural or a synthetic amino acid. Preferably, the amino acid(s) are all in their L-configuration. n is an integer in the range of 1-5, preferably in the range of 2-5. Thus, the peptide spacer contains 1-5 amino acids. Preferably, the peptide is a dipeptide (n=2) or tripeptide (n=3), most preferably the peptide spacer is a dipeptide. Although any peptide spacer may be used, preferably the peptide spacer is selected from Val-Cit, Val-Ala, Val-Lys, Val-Arg, AcLys-Val-Cit, AcLys-Val-Ala, Glu-Val-Ala, Asp-Val-Ala, Phe-Cit, Phe-Ala, Phe-Lys, Phe-Arg, Ala-Lys, Leu-Cit, Ile-Cit, Trp-Cit, Ala-Ala-Asn, Ala-Asn and Lys, more preferably Val-Cit, Val-Ala, Glu-Val-Ala, Val-Lys, Phe-Cit, Phe-Ala, Phe-Lys, Ala-Ala-Asn, more preferably Val-Cit, Val-Ala, Ala-Ala-Asn, most preferably Val-Cit or Val-Ala. In one embodiment, the peptide spacer is Val-Cit. In one embodiment, the peptide spacer is Val-Ala.

R¹⁷ represents the amino acid side chain, preferably selected from the side chains of alanine, cysteine, aspartic acid, glutamic acid, phenylalanine, glycine, histidine, isoleucine, lysine, acetyllysine, leucine, methionine, asparagine, pyrrolysine, proline, glutamine, arginine, serine, threonine, selenocysteine, valine, tryptophan, tyrosine and citrulline. Preferred amino acid side chains are those of Val, Cit, Ala, Lys, Arg, AcLys, Phe, Leu, lie, Trp, Glu, Asp and Asn, more preferably from the side chains of Val, Cit, Ala, GLu and Lys. Alternatively worded, R¹⁷ is preferably selected from CH₃ (Ala), CH₂CH(CH₃)₂ (Leu), CH₂CH₂CH₂NHC(O)NH₂ (Cit), CH₂CH₂CH₂CH₂NH₂ (Lys), CH₂CH₂CH₂NHC(O)CH₃ (AcLys), CH₂CH₂CH₂NHC(═NH)NH₂ (Arg), CH₂Ph (Phe), CH(CH₃)₂ (Val), CH(CH₃)CH₂CH₃ (Ile), CH₂C(O)NH₂ (Asn), CH₂CH₂C(O)OH (Glu), CH₂C(O)OH (Asp) and CH₂(1H-indol-3-yl) (Trp). Especially preferred embodiments of R¹⁷ are CH₃ (Ala), CH₂CH₂CH₂NHC(O)NH₂ (Cit), CH₂CH₂CH₂CH₂NH₂ (Lys), CH₂CH₂C(O)OH (Glu) and CH(CH₃)₂ (Val). Most preferably, R¹⁷ is CH₃ (Ala), CH₂CH₂CH₂NHC(O)NH₂ (Cit), CH₂CH₂CH₂CH₂NH₂ (Lys), or CH(CH₃)₂ (Val).

In an especially preferred embodiment, the peptide spacer may be represented by general structure (L³):

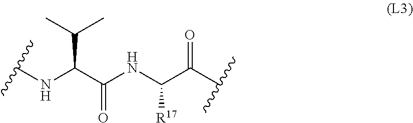

Herein, R¹⁷ is as defined above, preferably R¹⁷ is CH₃ (Val) or CH₂CH₂CH₂NHC(O)NH₂ (Cit).

The para-aminobenzyloxycarbonyl (PABC) derivative may be represented by general structure (L⁴):

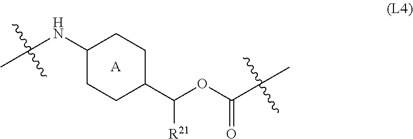

A is a 5- or 6-membered aromatic or heteroaromatic ring, preferably a 6-membered aromatic or heteroaromatic ring. Suitable 5-membered rings are oxazole, thiazole and furan. Suitable 6-membered rings are phenyl and pyridyl. In a preferred embodiment, A is 1,4-phenyl, 2,5-pyridyl or 3,6-pyridyl. Most preferably, A is 1,4-phenyl.

R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which are optionally substituted and optionally interrupted by one or more heteroatoms selected from O, S and NR²³ wherein R²³ is independently selected from the group consisting of hydrogen and C₁-C₄ alkyl groups. Preferably, R²² is C₃-C₁₀ (hetero)cycloalkyl or polyalkylene glycol. The polyalkylene glycol is preferably a polyethylene glycol or a polypropylene glycol, more preferably —(CH₂CH₂O)ₛH or —(CH₂CH₂CH₂O)ₛH. The polyalkylene glycol is most preferably a polyethylene glycol, preferably —(CH₂CH₂O)ₛH, wherein s is an integer in the range 1-10, preferably 1-5, most preferably s=1, 2, 3 or 4. More preferably, R²¹ is H or C(O)R²², wherein R²²=4-methyl-piperazine or morpholine. Most preferably, R²¹ is H.

Linkers (L¹, L² and L⁵)

Linkers, also referred to as linking units, are well known in the art and any suitable linker may be used. In the context of the present invention, the exatecan payload is chemically connected to the connecting group Z obtained by a metal-free click reaction or thiol ligation via linker L², and the connecting group Z is chemically connected to the antibody via linker L¹. Connecting group Z is chemically connected to (O)ₐ via linker L⁵. A linker, especially linker L², may contain one or more branch-points for attachment of multiple payloads to a single connecting group. Linker L¹ may be present (w=1) or absent (w=0). In case linker L¹ is absent, F is attached directly to the antibody. Preferably, w=0 in case the conjugation is via thiol ligation. Preferably, w=1 in case the conjugation is via a click reaction. Linker L⁵ may be present (r=1) or absent (r=0).

A linker may for example be selected from the group consisting of linear or branched C₁-C₂₀₀alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)_{y} and NR¹², wherein y is 0, 1 or 2, preferably y=2, and R¹² is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups. The linker may contain (poly)ethylene glycoldiamines (e.g. 1,8-diamino-3,6-dioxaoctane or equivalents comprising longer ethylene glycol chains), (poly)ethylene glycol or (poly)ethylene oxide chains, (poly)propylene glycol or (poly)propylene oxide chains and 1,z-diaminoalkanes wherein z is the number of carbon atoms in the alkane, and may for example range from 2-25.

In a preferred embodiment, linker L² (in particular Sp¹ if present) contains a polar group. Such a polar group may be selected from —(O)ₐ—C(O)—NH—S(O)₂—NR¹³— (as further defined below), —C(S(O)₃⁽⁻⁾)—, —C(C(O)₂⁽⁻⁾)—, —S(O)₂—, —P(O)₂⁽⁻⁾—, —O(CH₂CH₂O)ₜ—, —NR³⁰(CH₂CH₂NR³⁰)ₜ—, and the following two structures:

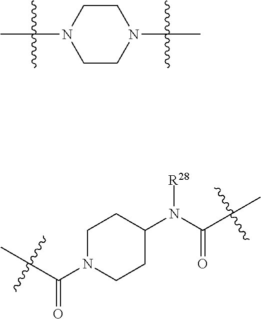

The polar group may also contain an amino acid, preferably selected from Arg, Glu, Asp, Ser and Thr. Herein, a and R¹³ are further defined below for structure (3). t is an integer in the range of integer in the range of 0-15, preferably 1-10, more preferably 2-5, most preferably t=2 or 4. Each R³⁰ is individually H, C₁₋₁₂ alkyl, C₁₋₁₂ aryl, C₁₋₁₂ alkaryl or C₁₋₁₂ aralkyl. Linker L² may contain more than one such polar group, such as at least two polar groups. The polar group may also be present in a branch of linker L² (in particular Sp¹ if present), which branches off a branching moiety as defined elsewhere. Preferable, a nitrogen or carbon atom is used as branching moiety. It is especially preferred to have a —O(CH₂CH₂O)ₜ— polar group present in a branch.

In a preferred embodiment, linker L² comprises a sulfamide group, preferably a sulfamide group according to structure (3):

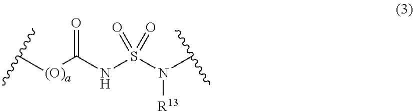

The wavy lines represent the connection to the remainder of the conjugate, typically to Z and to (NH—CR¹⁷—CO)ₙ, optionally via a spacer. Preferably, the (O)ₐC(O) moiety is connected to Z and the NR¹³ moiety to (NH—CR¹⁷—CO)ₙ. In case linker L² comprises a spacer moiety Sp¹, it is preferred that the sulfamide group according to structure (3) is comprised in spacer moiety Sp¹.

In structure (3), a=0 or 1, preferably a=1, and R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is a second occurrence of the exatecan payload connected to N via a spacer moiety (i.e. the nitrogen atom may be a branching moiety).

In a preferred embodiment, R¹³ is hydrogen, a C₁-C₂₀ alkyl group or R¹³ is a second occurrence of the exatecan payload connected to N via a spacer moiety. More preferably R¹³ is hydrogen, a C₁-C₁₀ alkyl group or a second occurrence of the exatecan payload connected to N via a spacer moiety. Herein, the alkyl group is optionally substituted and optionally interrupted by one or more heteroatoms selected from O, S and NR¹⁴, preferably O, wherein R¹⁴ is independently selected from the group consisting of hydrogen and C₁-C₄ alkyl groups. In a preferred embodiment, R¹³ is hydrogen. In another preferred embodiment, R¹³ is a C₁-C₂₀ alkyl group, more preferably a C₁-C₁₆ alkyl group, even more preferably a C₁-C₁₀ alkyl group, wherein the alkyl group is optionally interrupted by one or more 0-atoms, and wherein the alkyl group is optionally substituted with an —OH group, preferably a terminal —OH group. In this embodiment it is further preferred that R¹³ is a (poly)ethylene glycol chain comprising a terminal —OH group. In another preferred embodiment, R¹³ is a second occurrence of the exatecan payload connected to N via a spacer moiety. In another preferred embodiment, R¹³ is selected from the group consisting of hydrogen, methyl, ethyl, n-propyl, i-propyl, n-butyl, s-butyl and t-butyl, more preferably from the group consisting of hydrogen, methyl, ethyl, n-propyl and i-propyl, and even more preferably from the group consisting of hydrogen, methyl and ethyl. Yet even more preferably, R¹³ is hydrogen or a second occurrence of the exatecan payload connected to N via a spacer moiety, and most preferably R¹³ is hydrogen.

The antibody-drug conjugate according to the invention may comprises two groups of formula (3), which are preferably both contained in L², more preferably both in Sp¹. Typically, there is a spacer connecting both occurrences of the groups of formula (3), such as a linker as defined herein. Preferably, this is a PEG spacer defined as (CH₂CH₂O)ₘ, wherein m is an integer in the range of 1-10, preferably in the range of 2-6, most preferably m is 2 or 4.

In one embodiment, linker L², preferably spacer Sp¹, contains structure (L³):

(O)ₐC(O)NHS(O)₂NH—(CH₂CH₂O)ₘ—C(O)—(NHS(O)₂)ₚN* (L³)

Herein, a and m are as defined above, and p is 0 or 1. N* may represent the nitrogen atom of the peptide spacer, or a branching moiety.

### Branching Moiety

In a preferred embodiment, the linker of the conjugate according to the invention contains a branching moiety. A “branching moiety” in the context of the present invention refers to a moiety that is embedded in a linker connecting three moieties. In other words, the branching moiety comprises at least three bonds to other moieties, typically one bond to antibody AB, connecting to Z, one bond to the exatecan payload and one bond to a second exatecan payload. The branching moiety is preferably embedded in linker L². Any moiety that contains at least three bonds to other moieties is suitable as branching moiety in the context of the present invention. In a preferred embodiment, the branching moiety BM is selected from a carbon atom, a nitrogen atom, a phosphorus atom, a (hetero)aromatic ring, a (hetero)cycle or a polycyclic moiety. Most preferably, the branching moiety is a nitrogen atom.

It is thus preferred that linker L² contains a branching moiety, preferably via a nitrogen atom, such that two exatecan payloads are connected to a single moiety Z. In an especially preferred embodiment, L² has structure (2):

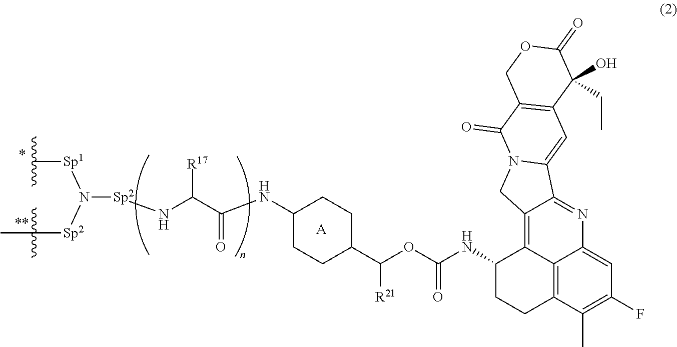

Herein, the wavy bond labeled with * is connected to Z and the wavy bond labeled with ** is connected to NH. Sp¹ and Sp² are each individually spacer moieties. Variables n, A, R¹⁷ and R²¹ are defined elsewhere, which equally applies to this embodiment. The two linker-exatecan moieties connected to the branching nitrogen atom may be the same or different. Preferably, they are the same, i.e. each occurrence of Sp² is the same, each occurrence of (NH—CR¹⁷—CO)ₙ is the same, each occurrence of A is the same and each occurrence of R²¹ is the same.

### Connecting Group Z

Z is a connecting group. The term “connecting group” refers to a structural element connecting one part of the conjugate and another part of the same bioconjugate. In (1), Z connects antibody AB with the exatecan payload, via a linker. Connecting group Z is a moiety is obtainable by a metal-free click reaction or by thiol ligation. As the skilled person understands, the exact nature of Z depends on the nature of F and Q. Preferred embodiments for Q and F are defined further below.

In a first preferred embodiment, connecting group Z is obtainable by a metal-free click reaction. Herein, connecting group Z may preferably contain a triazole moiety, an isoxazole moiety, a dihydroisoxazole moiety, a bicyclo[2.2.2]octa-5,7-diene-2,3-dione moiety, a bicyclo[2.2.2]octa-5-ene-2,3-dione moiety, a 7-thiabicyclo[2.2.1]hepta-2,5-diene-7,7-dioxide moiety, a 7-thiabicyclo[2.2.1]hept-2-ene-7,7-dioxide moiety, a pyrazole moiety, a pyridine moiety, a dihydropyridine moiety, a pyridazine moiety or a dihydropyridazine moiety. Preferred structures for the connecting group Z comprise moieties (Z1)-(Z8) depicted here below.

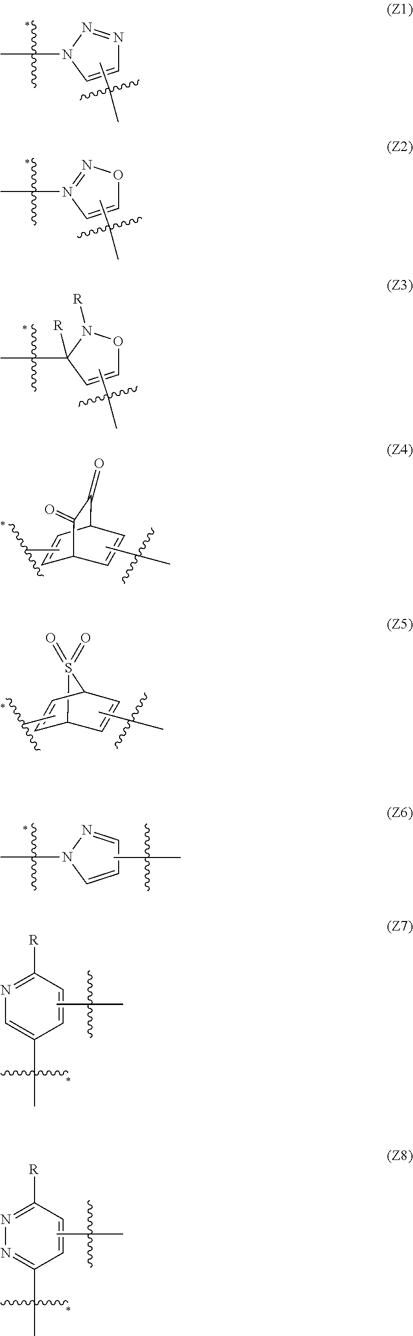

Herein, functional groups R in (Z3), (Z7) and (Z8) may be selected from hydrogen, C₁-C₂₄ alkyl groups, C₂-C₂₄ acyl groups, C₃-C₂₄ cycloalkyl groups, C₂-C₂₄ (hetero)aryl groups, C₃-C₂₄ alkyl(hetero)aryl groups, C₃-C₂₄ (hetero)arylalkyl groups and C₁-C₂₄ sulfonyl groups, each of which (except hydrogen) may optionally be substituted and optionally be interrupted by one or more heteroatoms selected from O, S and NR³² wherein R³² is independently selected from the group consisting of hydrogen and C₁-C₄ alkyl groups. The wavy bond labelled with an * is connected to L¹ and the other wavy bond to L². The skilled person understands which R groups may be applied for each of the connecting groups Z. For example, the R group connected to the nitrogen atom of (Z3) may be selected from alkyl or aryl as defined above, and the R group connected to the carbon atom of (Z3) may be selected from hydrogen, alkyl, aryl, acyl and sulfonyl as defined above.

In an especially preferred embodiment, Q contains a cyclic alkyne moiety and F is an azide, and Z contains a triazole moiety that is formed by 1,3-dipolar cycloaddition of the alkyne moiety with the azide moiety. In a further especially preferred embodiment, Z is according to any one of structures (Z36)-(Z40) defined here below, wherein the wavy bond labelled with an * is connected to L¹ and the other wavy bond to L².

In an especially preferred embodiment, connecting group Z is according to structure (Z37):

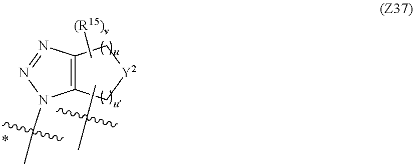

### Herein:

R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S or NR³¹, wherein each R³¹ individually is R¹⁵ or a second occurrence of the exatecan payload connected via a spacer moiety;
u is 0, 1, 2, 3, 4 or 5;
u′ is 0, 1, 2, 3, 4 or 5, wherein u+u′=4, 5, 6, 7 or 8;
v=an integer in the range 8-16.

In a preferred embodiment, u+u′=4, 5 or 6, more preferably u+u′=5. Typically, v=(u+u′)×2 or [(u+u′)×2]-1. In a preferred embodiment, v=8, 9 or 10, more preferably v=9 or 10, most preferably v=10.

In an especially preferred embodiment, connecting group Z is according to structure (Z38):

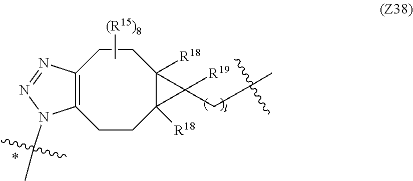

### Herein:

R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₇-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of the exatecan payload connected via a spacer moiety; and
I is an integer in the range 0 to 10.

In a preferred embodiment of the reactive group according to structure (Z38), R¹⁵ is independently selected from the group consisting of hydrogen, halogen, —OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the reactive group according to structure (Z38), R¹⁸ is independently selected from the group consisting of hydrogen, C₁-C₆ alkyl groups, most preferably both R¹⁸ are H. In a preferred embodiment of the reactive group according to structure (Z38), R¹⁹ is H. In a preferred embodiment of the reactive group according to structure (Z38), I is 0 or 1, more preferably I is 1.

In an especially preferred embodiment, connecting group Z is according to structure (Z39):

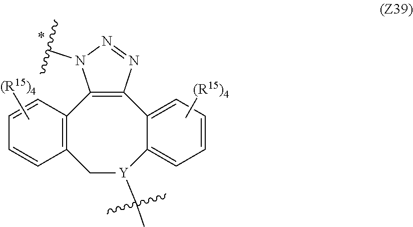

### Herein:

R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

In a preferred embodiment of the reactive group according to structure (Z39), R¹⁵ is independently selected from the group consisting of hydrogen, halogen, —OR¹⁶, —S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃⁽⁻⁾. In a preferred embodiment of the reactive group according to structure (Z39), Y is N or CH, more preferably Y=N.

In an especially preferred embodiment, connecting group Z is according to structure (Z36):

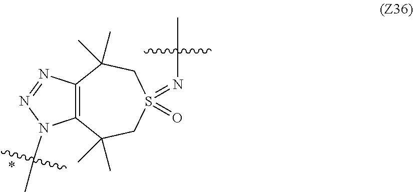 [SMILES: *N=S1(=O)CC(C)(C)c2nnn(*)c2C(C)(C)C1]

Most preferably, connecting group Z is according to structure (Z38), wherein R¹⁵, R¹⁸ and R¹⁹ are all H and I is 1.

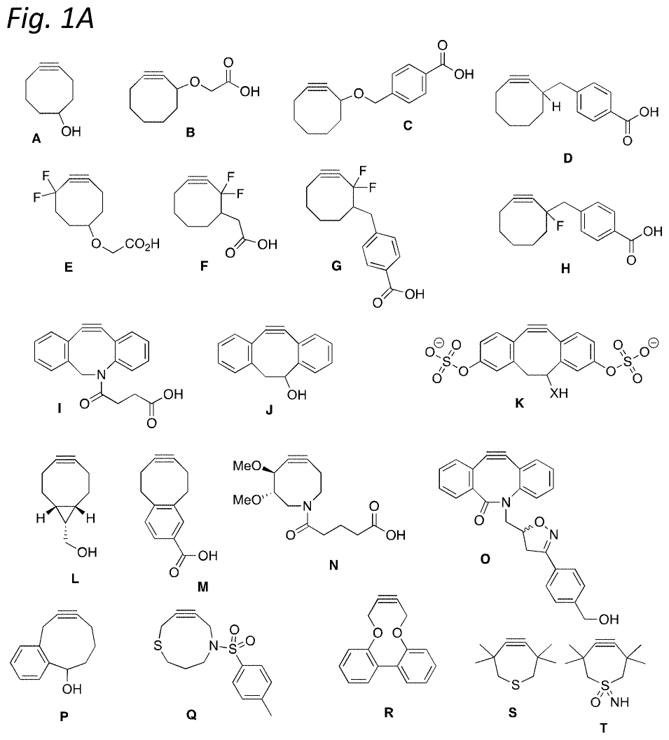

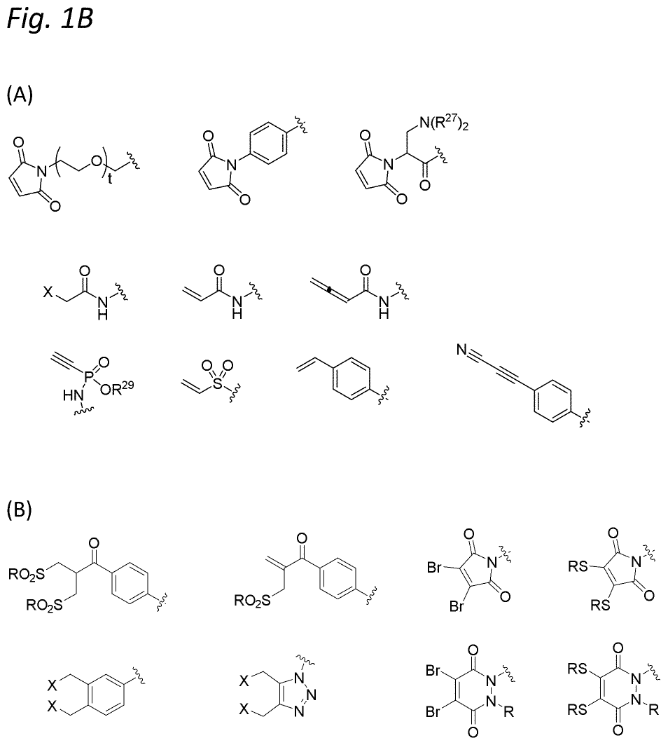

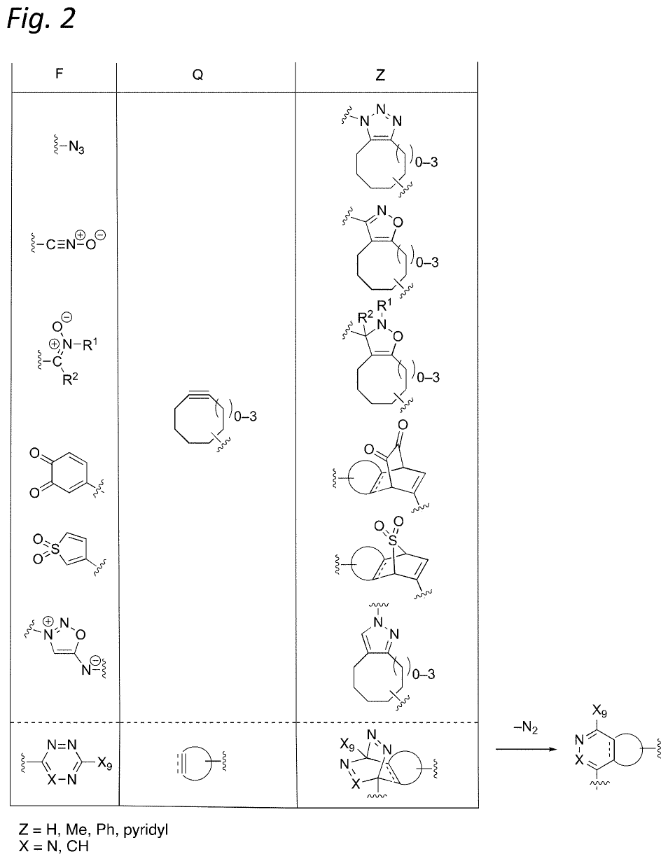

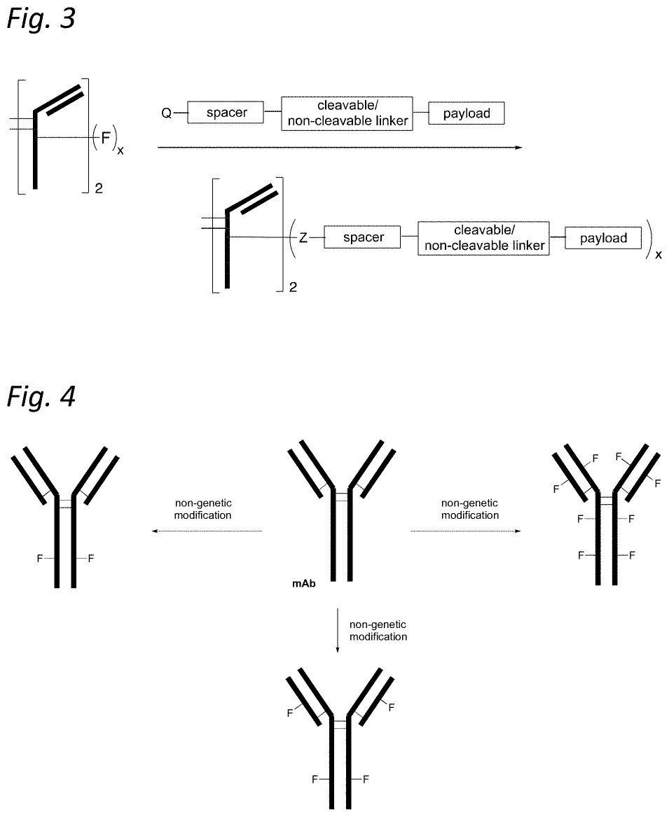

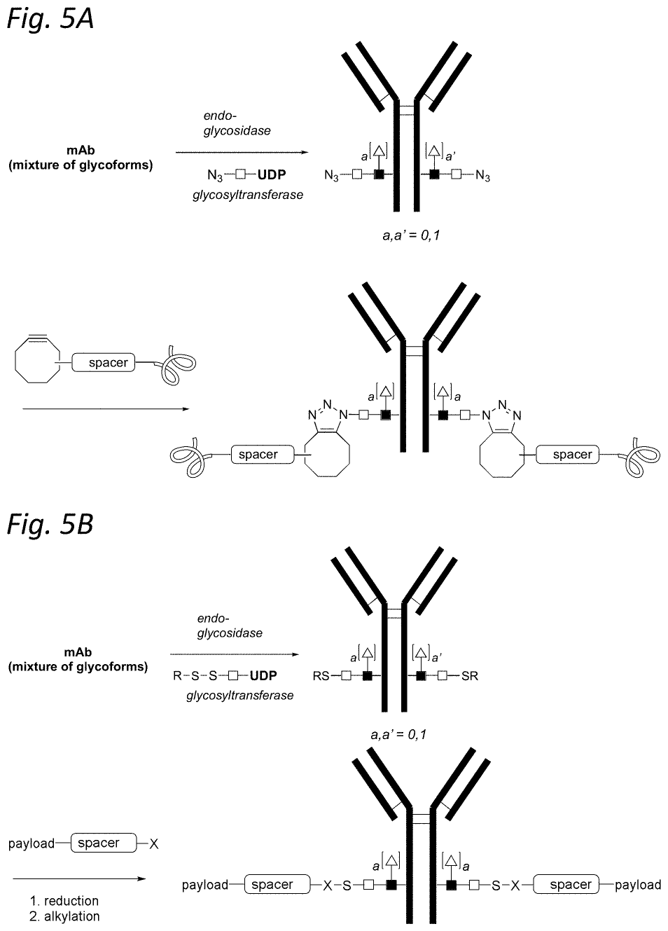

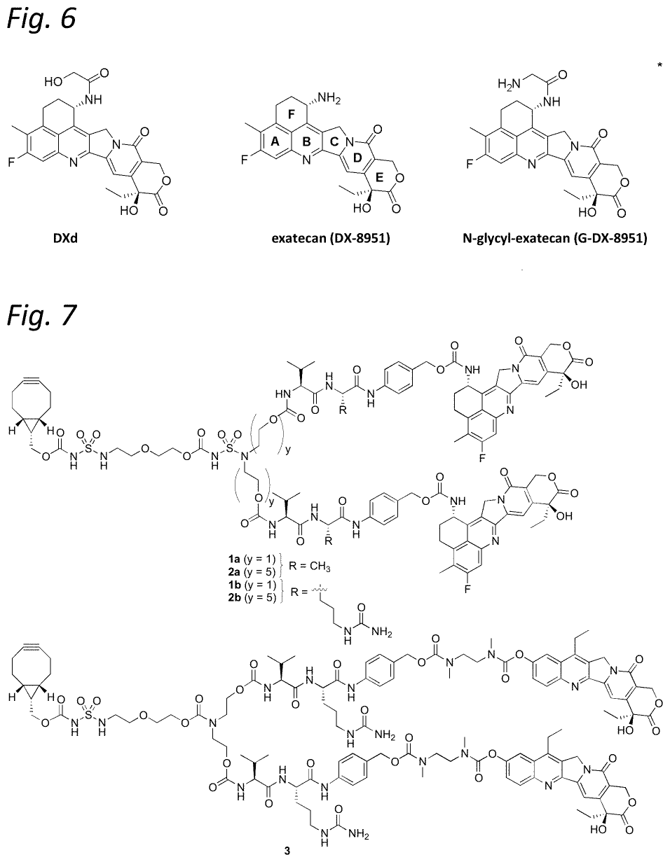

In a second preferred embodiment, connecting group Z is obtainable by thiol ligation. Herein, connecting group Z has one connection to L² and at least one connection to AB, possibly via L¹. One connection group Z may also have two connections to AB, in case a cross-linking thiol-reactive probe Q is used (see FIG. 1B). Preferably, L¹ is absent (w=0) and Z is connected directly to AB. In a preferred embodiment, connection group Z comprises a succinimidyl ring or its ring-opened succinic acid amide derivative. Preferred options for connection group Z, which contain one connection to AB, comprise a moiety selected from (Z10)-(Z20) depicted here below. Preferred options for connection group Z, which contain two connections to AB, comprise a moiety selected from (Z11)-(Z23) depicted here below.

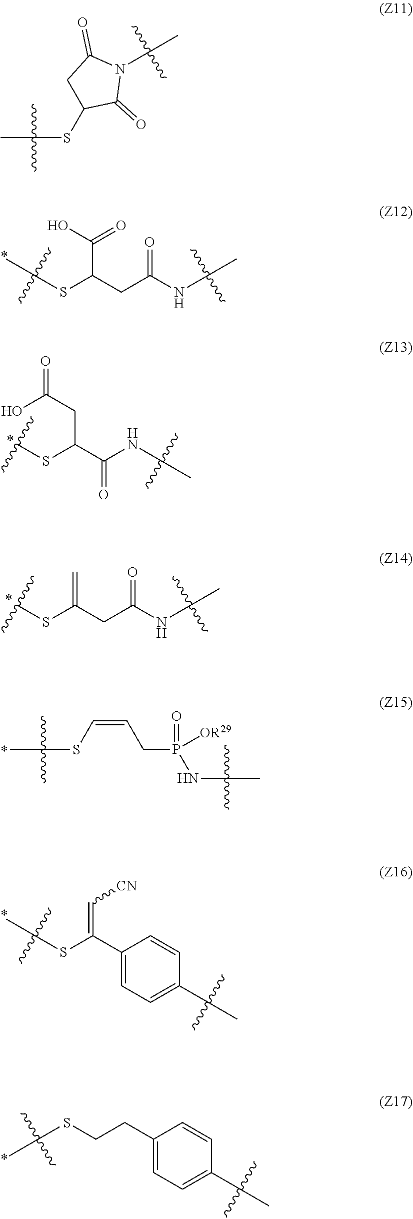

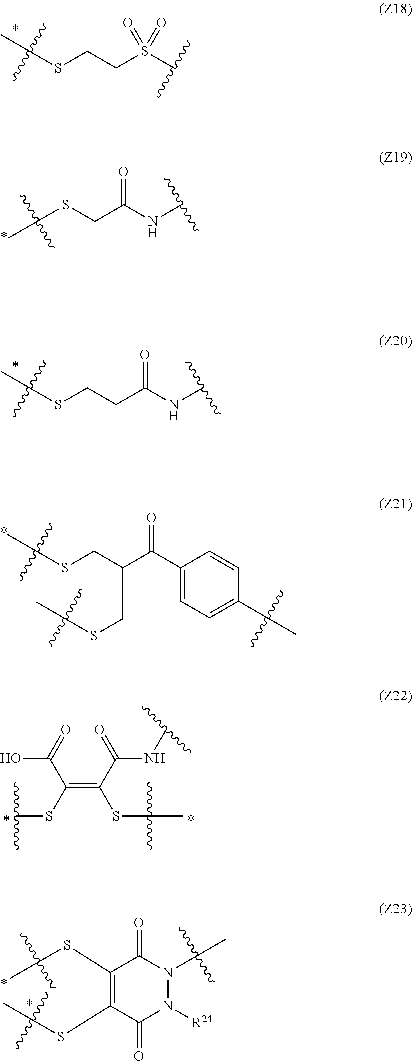

Herein, the wavy bond(s) labelled with an * is connected to AB, optionally via L¹, and the other wavy bond to L². In addition, the following applies:
R²⁴ is H or C₁₋₁₂ alkyl, preferably H or C₁₋₆ alkyl;
R²⁹ is C₁₋₁₂ alkyl, preferably C₁₋₄ alkyl, most preferably ethyl.

### Mode of Conjugation

The conjugates according to the invention can be prepared by any mode of conjugation. The conjugation reaction is a thiol ligation or a metal-free click reaction. The nature of the modified antibody AB-((L¹)_{w}-F)ₓ, in particular the nature of L¹, w and x, depends on the mode of conjugation employed. x refers to the number of attachment points (conjugation sites) of (L¹)_{w} on the antibody and is an integer in the range of 1-8. In case Z (in the conjugate) or F (in the modified antibody) is directly attached to the antibody, L¹ is absent and w=0. In this embodiment, the modified antibody may also be referred to as AB-(F)ₓ.

x represents the amount of probes F present on the antibody, optionally via linker L¹, and is an integer in the range of 1-8. Preferably, x is an integer in the range of 1-6, more preferably x=1, 2, 3 or 4, even more preferably x=1 or 2, most preferably x=2. Typically, x refers to the average number of moieties Z connected to the antibody. In a preferred embodiment, especially in case the conjugation is by click reaction, the conjugate according to the invention normally has the same amount of moieties Z connected to the antibody, although the conjugation reaction may at times be slightly incomplete. Notably, each linker L² may contain more than one exatecan payload, such as 1 or 2 payload molecules per linker L².

In a preferred embodiment, w=1 and the mode of conjugation involves conjugation via the glycan of the antibody, preferably via an N-glycosylation site. Preferably, the modified antibody comprises one or more glycans with structure -GlcNAc(Fuc)_{d}-(G)ₑ-Su-F, wherein e is an integer in the range of 0-20; Su is a monosaccharide; G is a monosaccharide moiety; GlcNAc is an N-acetylglucosamine moiety; Fuc is a fucose moiety; and d is 0 or 1. In other words, L¹ is preferably represented by -GlcNAc(Fuc)_{d}-(G)ₑ-Su- wherein GlcNAc(Fuc)_{d} is attached to the peptide part of the antibody and Su is attached to Z or F. Herein, GlcNAc is the core N-acetylglucosamine moiety which is typically present in the glycan structure of antibodies. Herein, the core N-acetylglucosamine moiety refers to the N-acetylglucosamine moiety that is directly attached to the peptide chain of the antibody. This core N-acetylglucosamine moiety is optionally fucosylated (d is 0 or 1), which is a common feature of antibodies.

(G)ₑ represents the glycoform of the antibody. The present invention may be applied to antibodies of any glycoform. Typical monosaccharides that are present in glycans, and from among which G may be selected, include glucose, galactose, mannose, fucose, N-acetylglucosamine, N-acetylgalactosamine, N-acetylneuraminic acid and xylose. (G)ₑ may thus be a linear or branched oligosaccharide, comprising e monosaccharide moieties. Typical glycans have e in the range of 4-16, preferably 6-10. In a preferred embodiment, the antibody is trimmed and e=0. Such trimming may be performed by an endoglycosidase enzyme such as EndoS. Conjugation via the glycan preferably employs an N-glycosylation site, more preferably an N-glycosylation site connected to an asparagine amino acid of the antibody, most preferably to the conserved glycosylation site at amino acid N297 of the antibody.

Su is a monosaccharide containing F. Su is preferably selected from the group consisting of galactose (Gal), mannose (Man), glucose (Glc), N-acetylglucosamine (GlcNAc), N-acetylgalactosamine (GalNAc), N-acetylneuraminic acid or sialic acid (Sial) and fucose (Fuc). Su is preferably a glucose or galactose derivative, more preferably a galactose derivative, most preferably N-acetylgalactosamine.

In an alternative preferred embodiment, w=0 and the conjugation site at the antibody is a cysteine residue, preferably wherein the cysteine residue is naturally present in the antibody, optionally after reduction of a disulfide bond as known in the art, or wherein the cysteine residue is engineered, preferably by substitution of an amino acid by cysteine, cysteine insertion or introduction of a single cysteine residue or a peptide fragment containing a cysteine residue at the N- or C-terminus.

AB represents the antibody. The skilled person understands that the present invention can be applied to any antibody. Preferably, the antibody targets HER2.

### Preferred Conjugates

Preferred conjugates according to the present invention have structure (1f):

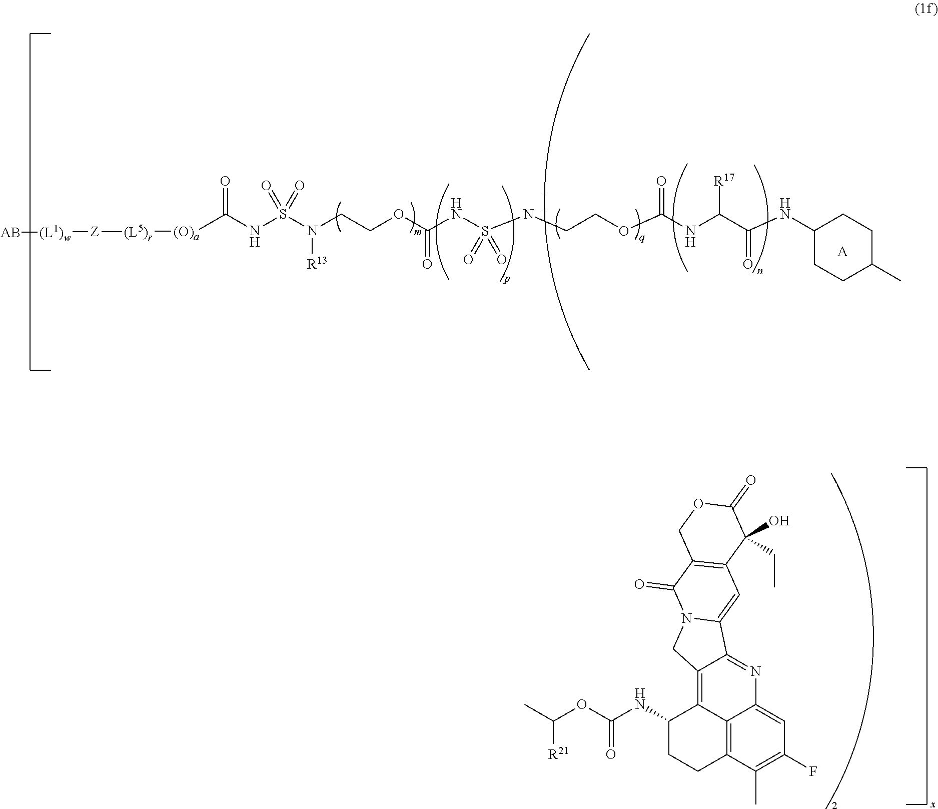

Herein, the exact nature of L² is further specified, and contains a branching moiety N. AB, L¹, Z, A, a, R¹³, R²¹, x, n, R¹⁷, m and p are defined elsewhere herein, L⁵ is a linker, r is 0 or 1, and q is an integer in the range of 0-10. Preferably, linker L⁵ is absent or CH₂. Preferably, m is an integer in the range of 1-10; q is an integer in the range of 1-4; and p is 1. Preferably, n is 1, 2 or 3. Even more preferably:
AB is an antibody;
L¹ is a linker;
Z is a connecting group according to structure (Z38), preferably wherein R¹⁵, R¹⁸ and R¹⁹ are all H and I is 1;
a=1
R¹³ is hydrogen;
r=0 and L⁵ is absent;
each (NH-CH(R¹⁷)—C(O))ₙ is Val-Ala or Val-Cit;
m is 2 or 4, preferably m=2
p is 0 or 1, preferably p=1
each q is 2 or 4, preferably each q=2
each A is 1,4-phenyl;
x is an integer in the range of 2-4, preferably x=2;
each R²¹ is H.

Preferred conjugates according to the present invention have structure (1a):

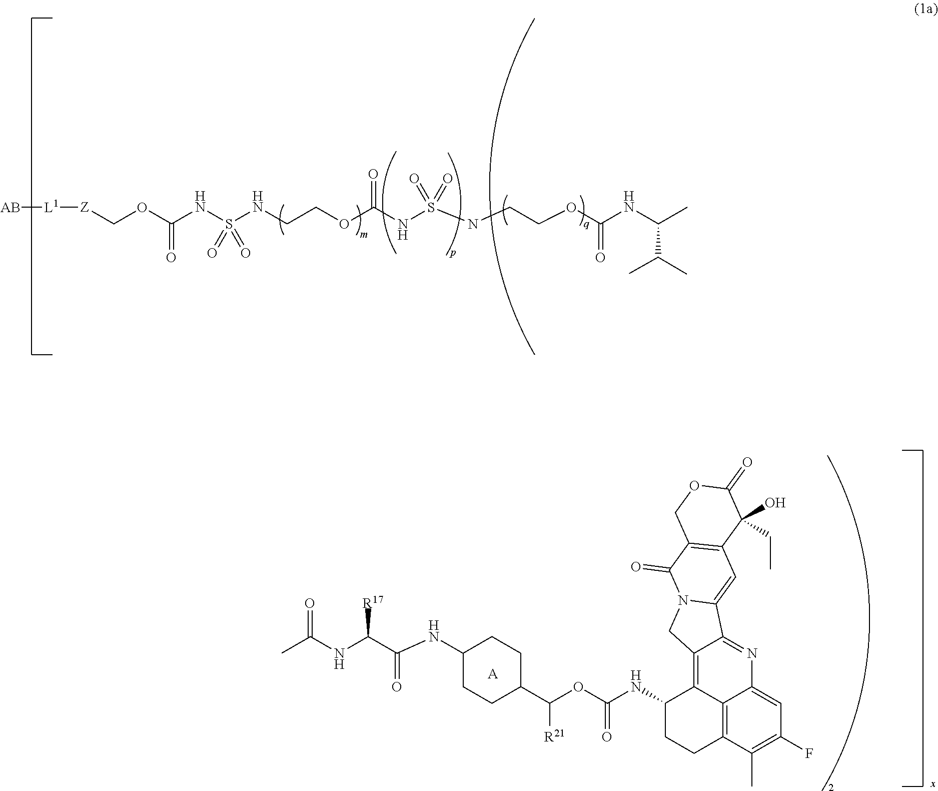

Herein, the exact nature of L² is further specified, and contains a branching moiety N. AB, L¹, Z, A, R²¹, x, R¹⁷, m and p are defined elsewhere herein, and q is an integer in the range of 0-10. Preferably, R¹⁷ is CH₃ or CH₂CH₂CH₂NHC(O)NH₂; m is an integer in the range of 1-10; q is an integer in the range of 1-4; and p is 1. Even more preferably:
AB is an antibody;
L¹ is a linker;
Z is a connecting group according to structure (Z38), preferably wherein R¹⁵, R¹⁸ and R¹⁹ are all H and I is 1;
each R¹⁷ is individually CH₃ or CH₂CH₂CH₂NHC(O)NH₂, preferably each R¹⁷ is CH₃;
m is 2 or 4, preferably m=2
p is 0 or 1, preferably p=1
each q is 2 or 4, preferably each q=2
each A is 1,4-phenyl;
x is an integer in the range of 2-4, preferably x=2;
each R²¹ is H.

Preferred conjugates according to the present invention have structure (1b):

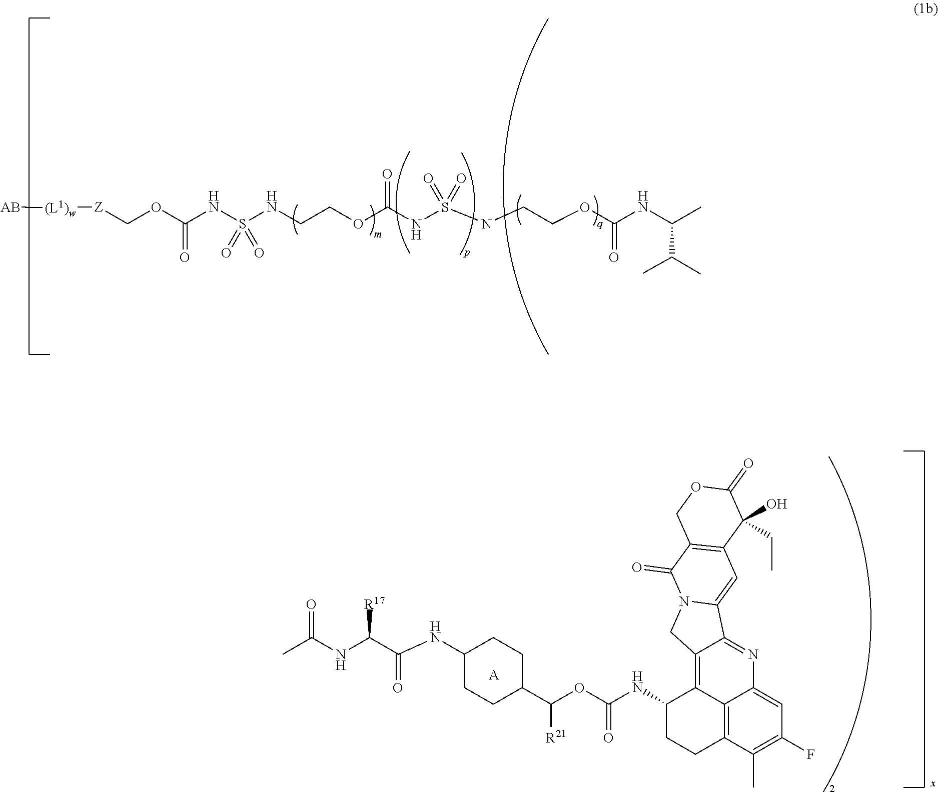

Herein, the exact nature of L² is further specified, and contains a branching moiety N. AB, L¹, w, Z, A, R²¹, x, R¹⁷, m and p are defined elsewhere herein, and q is an integer in the range of 0-10. Preferably, R¹⁷ is CH₃ or CH₂CH₂CH₂NHC(O)NH₂; m is an integer in the range of 1-10; q is an integer in the range of 1-4; and p is 1. Even more preferably:
AB is an antibody;
L¹ is a linker;
w is 0 or 1;
Z is a connecting group according to structure (Z1);
each R¹⁷ is individually CH₃ or CH₂CH₂CH₂NHC(O)NH₂, preferably each R¹⁷ is CH₃;
m is 2 or 4, preferably m=2
p is 0 or 1, preferably p=1
q is 2 or 4, preferably q=2
each A is 1,4-phenyl;
x is an integer in the range of 2-4, preferably x=2;
each R²¹ is H.

Further preferred conjugates according to the present invention have structure (1c):

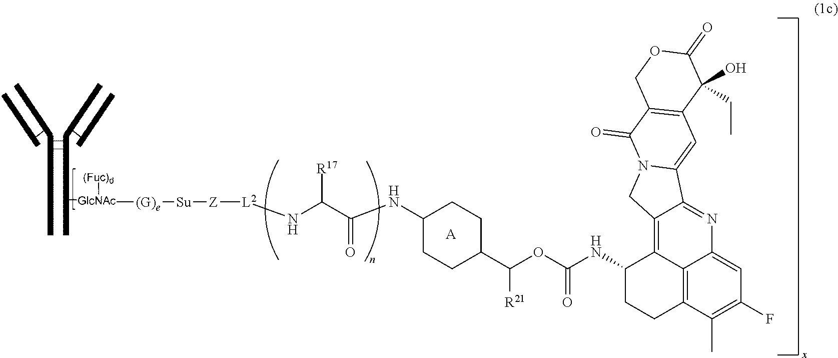

Herein, the exact nature of L¹ and the mode of conjugation to the antibody is further specified. Z, L², A, R²¹, x and R¹⁷ are defined elsewhere herein. Additionally, e is an integer in the range of 0-20; Su is a monosaccharide; G is a monosaccharide moiety; GlcNAc is an N-acetylglucosamine moiety; Fuc is a fucose moiety; and d is 0 or 1.

Especially preferred conjugates according to the present invention combine the mode of conjugation and L¹ definition of structure (1c) and the definition of L² of structure (1f), and thus have structure (1g):

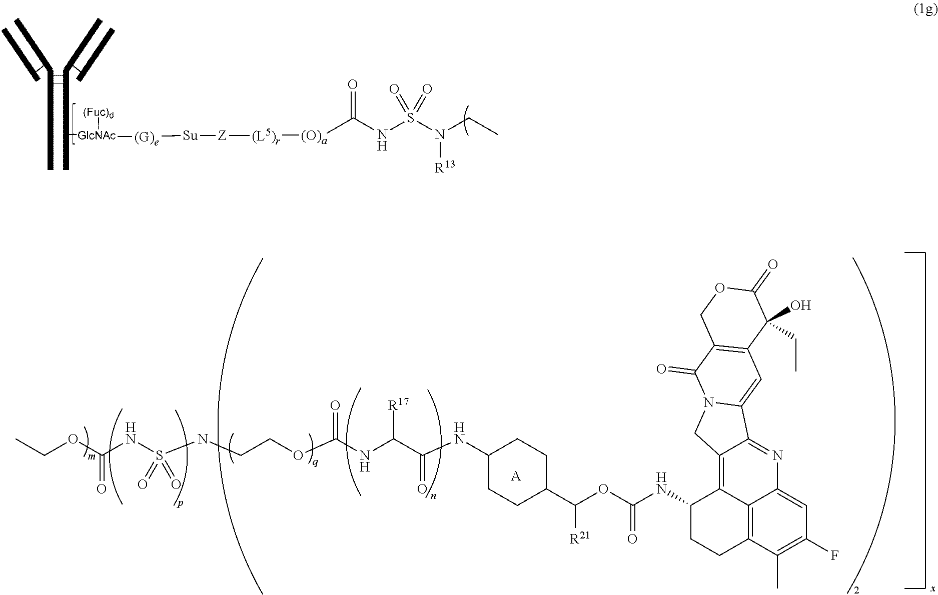

Herein, all of e, Su, G, GlcNAc, Fuc, d, Z, L⁵, r, m, p, q, A, a, R¹³, R²¹, n, R¹⁷, and x are as defined above, including preferred embodiments thereof.

In an especially preferred embodiment, the conjugate according to the invention has structure (1d), wherein
d is 0 or 1;
e is 0 and G is absent;
Su is GalNAc;
Z is a connecting group according to structure (Z38), preferably wherein R¹⁵, R¹⁸ and R¹⁹ are all H and I is 1;
a=1
R¹³ is hydrogen;
r=0 and L⁵ is absent;
each (NH—CH(R¹⁷)—C(O))ₙ is Val-Ala or Val-Cit;
m is 2 or 4, preferably m=2
p is 0 or 1, preferably p=1
each q is 2 or 4, preferably each q=2
each A is 1,4-phenyl;
x is an integer in the range of 2-4, preferably x=2;
each R²¹ is H.

Especially preferred conjugates according to the present invention combine the mode of conjugation and L¹ definition of structure (1c) and the definition of L² of structure (1a), and thus have structure (1d):

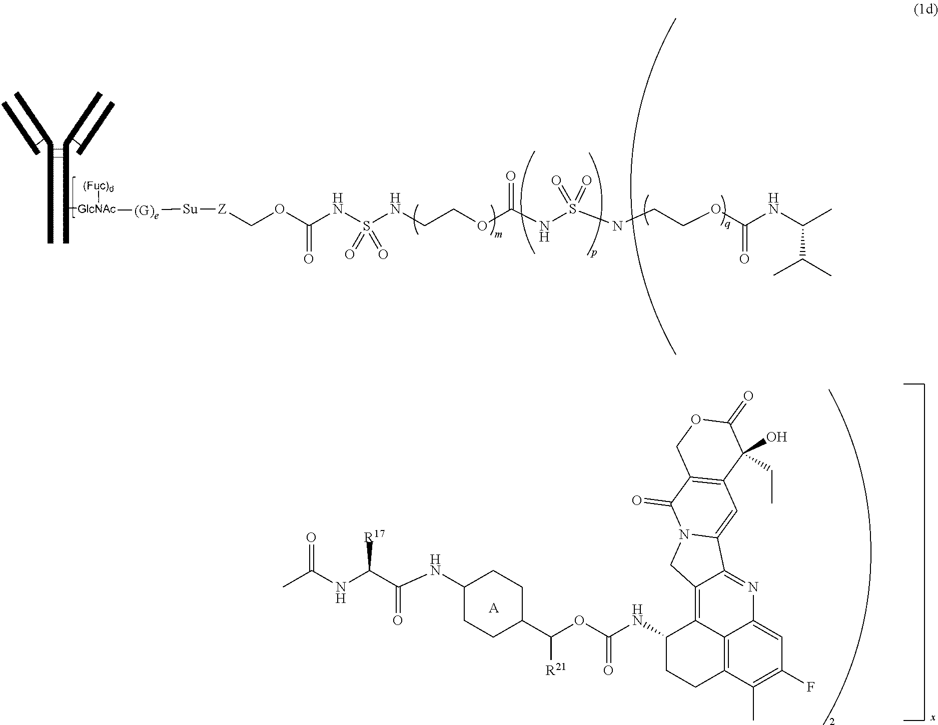

Herein, all of e, Su, G, GlcNAc, Fuc, d, Z, m, p, q, R¹⁷, A, R²¹ and x are as defined above, including preferred embodiments thereof.

In an especially preferred embodiment, the conjugate according to the invention has structure (1d), wherein
d is 0 or 1;
e is 0 and G is absent;
Su is GalNAc;
Z is a connecting group according to structure (Z38), preferably wherein R¹⁵, R¹⁸ and R¹⁹ are all H and I is 1;
each R¹⁷ is individually CH₃ or CH₂CH₂CH₂NHC(O)NH₂, preferably each R¹⁷ is CH₃;
m is 2 or 4, preferably m=2
p is 0 or 1, preferably p=1
each q is 2 or 4, preferably each q=2
each A is 1,4-phenyl;
x is an integer in the range of 2-4, preferably x=2;
each R²¹ is H.

Especially preferred conjugates according to the present invention combine the mode of conjugation and L¹ definition of structure (1c) and the definition of L² of structure (1b), and thus have structure (1e):

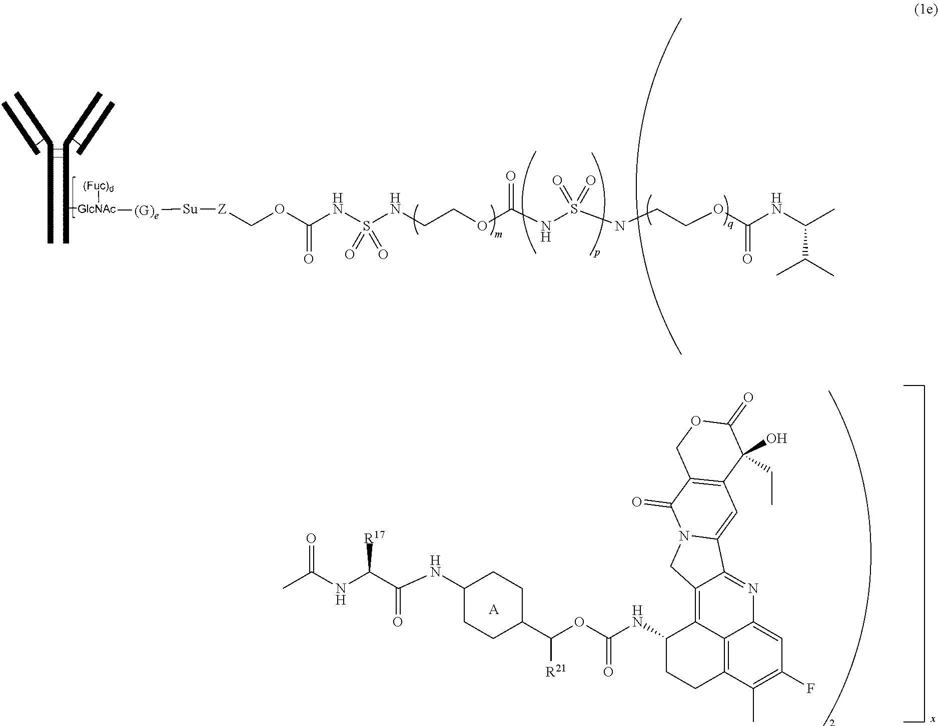

Herein, all of e, Su, G, GlcNAc, Fuc, d, Z, m, p, q, R¹⁷, A, R²¹ and x are as defined above, including preferred embodiments thereof.

In an especially preferred embodiment, the conjugate according to the invention has structure (1e), wherein
d is 0 or 1;
e is 0 and G is absent;
Su is GalNAc;
Z is a connecting group according to structure (Z1);
each R¹⁷ is individually CH₃ or CH₂CH₂CH₂NHC(O)NH₂, preferably each R¹⁷ is CH₃;
m is 2 or 4, preferably m=2;
p is 0 or 1, preferably p=1;
q is 2 or 4, preferably q=2;
each A is 1,4-phenyl;
x is an integer in the range of 2-4, preferably x=2;
each R²¹ is H.

In an especially preferred embodiment, the conjugates according to the present invention has structure (1h):

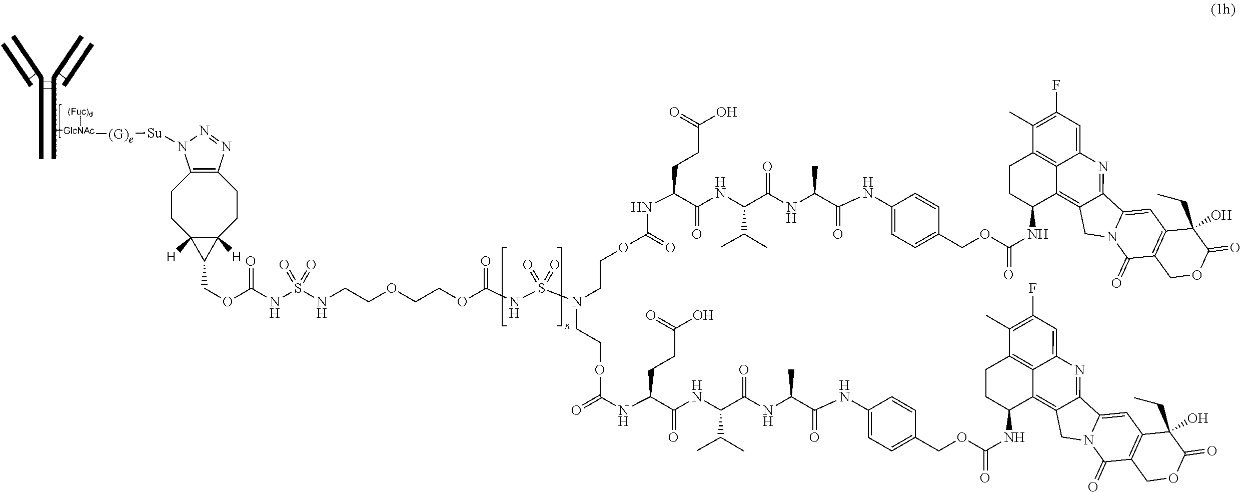

Herein, e, Su, G, GlcNAc, Fuc and d are as defined above, including preferred embodiments thereof, and n=0 or 1. Structure (1h) corresponds to conjugates of compounds (31a), with n=0, and (32a), with n=1, in the examples. Most preferably, e=0 and Su is N-acetylgalactosamine.

### Process for the Synthesis of the Antibody-Drug Conjugate

In a second aspect, the invention concerns a process for preparing the antibody-drug conjugate according to the invention. The process according to the invention comprises reacting (i) a modified antibody of structure AB-((L¹)_{w}-F)ₓ with (ii) a linker-drug construct according to structure (5). The reaction is a conjugation reaction, which forms a covalent attachment between the exatecan payload and the antibody. The reaction is a metal-free click reaction or a thiol ligation, and forms an antibody-drug conjugate wherein the drug is covalently attached to the antibody via connecting group Z that is formed by a metal-free click reaction between Q and F or a thiol ligation between Q and F.

In the process according to the invention, the modified antibody of structure AB-((L¹)_{w}-F)ₓ, wherein AB is an antibody, L¹ is a linker, w is 0 or 1, F is a click probe capable of reacting with Q in a metal-free click reaction or a thiol or precursor thereof capable of reacting with Q in a thiol ligation and x is an integer in the range of 1-8. Upon reacting F with Q of the linker-drug construct according to structure (5) via a metal-free click reaction or a thiol ligation, connecting group Z is formed. In one embodiment, the modified antibody may be referred to as AB-(F)ₓ. Methods of preparing modified antibody are known in the art, e.g. from WO 2014/065661, WO 2016/170186 and WO 2016/053107, which are incorporated herein by reference. From the same documents, the conjugation reaction between the modified glycoprotein and a linker-drug construct comprising a cytotoxin and a click probe is known to the skilled person.

In the process according to the invention, the linker-drug construct has structure (5):

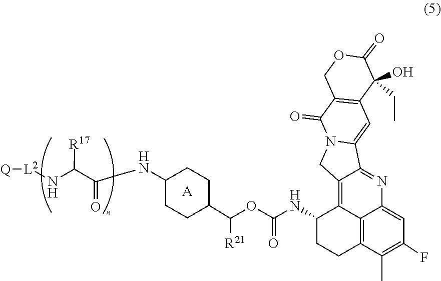

wherein:
   L² is a linker;
   Q is a click probe capable of performing a metal-free click reaction or a thiol-reactive probe;
   each R¹⁷ is individually an amino acid side chain;
   n is an integer in the range of 1-5;
   A is a 5- or 6-membered aromatic or heteroaromatic ring;
   x is an integer in the range of 1-8;
   R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which are optionally substituted and optionally interrupted by one or more heteroatoms selected from O, S and NR²³ wherein R²³ is independently selected from the group consisting of hydrogen and C₁-C₄ alkyl groups.

### Metal-Free Click Reaction

Metal-free click reactions are well-known in the art (see e.g. from WO 2014/065661 and Nguyen and Prescher, Nature rev. 2020, doi: 10.1038/s41570-020-0205-0, both incorporated by reference) and may typically take the form of a 1,3-dipolar cycloaddition or (4+2) cycloaddition. The alkyne-azide cycloaddition may be strain-promoted (e.g. a strain-promoted alkyne-azide cycloaddition, SPAAC). In a preferred embodiment, the bioconjugation reaction is a metal-free strain-promoted cycloaddition, most preferably metal-free strain-promoted alkyne-azide cycloaddition. In a preferred embodiment, conjugation is accomplished via a cycloaddition, such as a (4+2) cycloaddition or a 1,3-dipolar cycloaddition, preferably the 1,3-dipolar cycloaddition.

A typical (4+2) cycloaddition is the Diels-Alder reaction, wherein Q is a diene or a dienophile. As appreciated by the skilled person, the term “diene” in the context of the Diels-Alder reaction refers to 1,3-(hetero)dienes, and includes conjugated dienes (R₂C═CR—CR═CR₂), imines (e.g. R₂C═CR—N═CR₂ or R₂C═CR—CR═NR, R₂C═N—N═CR₂) and carbonyls (e.g. R₂C═CR—CR═O or O═CR—CR═O). Hetero-Diels-Alder reactions with N- and O-containing dienes are known in the art. Any diene known in the art to be suitable for (4+2) cycloadditions may be used as reactive group Q. Preferred dienes include tetrazines, 1,2-quinones and triazines. Although any dienophile known in the art to be suitable for (4+2) cycloadditions may be used as reactive group Q, the dienophile is preferably an alkene or alkyne group as described above, most preferably an alkyne group. For conjugation via a (4+2) cycloaddition, it is preferred that Q is a dienophile (and F is a diene), more preferably Q is or comprises an alkynyl group.

For a 1,3-dipolar cycloaddition, Q is a 1,3-dipole or a dipolarophile. Any 1,3-dipole known in the art to be suitable for 1,3-dipolar cycloadditions may be used as reactive group Q. Preferred 1,3-dipoles include azido groups, nitrone groups, nitrile oxide groups, nitrile imine groups and diazo groups. Although any dipolarophile known in the art to be suitable for 1,3-dipolar cycloadditions may be used as reactive groups Q, the dipolarophile is preferably an alkene or alkyne group, most preferably an alkyne group. For conjugation via a 1,3-dipolar cycloaddition, it is preferred that Q is a dipolarophile (and F is a 1,3-dipole), more preferably Q is or comprises an alkynyl group.

Thus, in a preferred embodiment, Q is selected from dipolarophiles and dienophiles.

### Click Probe Q

Click probe Q is used in the conjugation reaction to connect the linker-drug construct to the antibody of structure AB-(F)ₓ. Q is reactive towards click probe F in a metal-free click reaction. Such click probes are known in the art and include cyclic alkene, cyclic alkyne, azide, tetrazine, triazine, nitrone, nitrile oxide, nitrile imine, diazo compound, ortho-quinone, dioxothiophene and sydnone. Preferably, Q is a cyclic alkene or a cyclic alkyne moiety, most preferably Q is a cyclic alkyne moiety.

In an especially preferred embodiment, the click probe Q comprises a cyclic alkyne moiety. The alkynyl group may also be referred to as a (hetero)cycloalkynyl group, i.e. a heterocycloalkynyl group or a cycloalkynyl group, wherein the (hetero)cycloalkynyl group is optionally substituted. Preferably, the (hetero)cycloalkynyl group is a (hetero)cycloheptynyl group, a (hetero)cyclooctynyl group, a (hetero)cyclononynyl group or a (hetero)cyclodecynyl group. Herein, the (hetero)cycloalkynes may optionally be substituted. Preferably, the (hetero)cycloalkynyl group is an optionally substituted (hetero)cycloheptynyl group or an optionally substituted (hetero)cyclooctynyl group. Most preferably, the (hetero)cycloalkynyl group is a (hetero)cyclooctynyl group, wherein the (hetero)cyclooctynyl group is optionally substituted. Preferably, Q comprises a (hetero)cyclooctyne moiety according to structure (Q1) below. In a further preferred embodiment, the (hetero)cyclooctynyl group is according to structure (Q37), (Q38) or (Q39) as defined further below. Preferred examples of the (hetero)cyclooctynyl group include structure (Q2), also referred to as a DIBO group, (Q3), also referred to as a DIBAC group, or (Q4), also referred to as a BARAC group, (Q5), also referred to as a COMBO group, and (Q6), also referred to as a BCN group, all as shown below, wherein Y¹ is O or NR¹¹, wherein R¹¹ is independently selected from the group consisting of hydrogen, a linear or branched C₁-C₁₂ alkyl group or a C₄-C₁₂ (hetero)aryl group. The aromatic rings in (Q2) are optionally O-sulfonylated at one or more positions, whereas the rings of (Q3) and (Q4) may be halogenated at one or more positions. A particularly preferred cycloalkynyl group is a bicyclo[6.1.0]non-4-yn-9-yl] group (BCN group), which is optionally substituted. Preferably, the bicyclo[6.1.0]non-4-yn-9-yl] group is according to formula (Q6) as shown below, wherein V is (CH₂)ₗ and I is an integer in the range of 0 to 10, preferably in the range of 0 to 6. More preferably, I is 0, 1, 2, 3 or 4, more preferably I is 0, 1 or 2 and most preferably I is 0 or 1. In the context of group (Q6), I is most preferably 1.

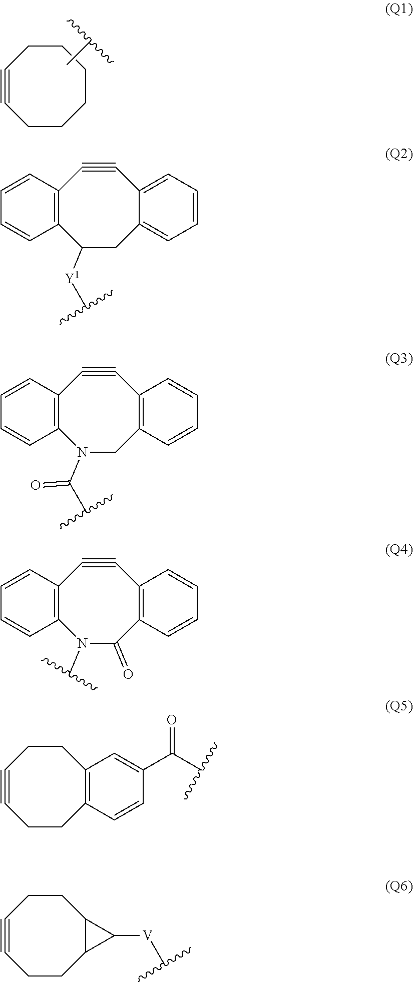

In a further preferred embodiment, the click probe Q is selected from the group consisting of (Q7)-(Q21) depicted here below.

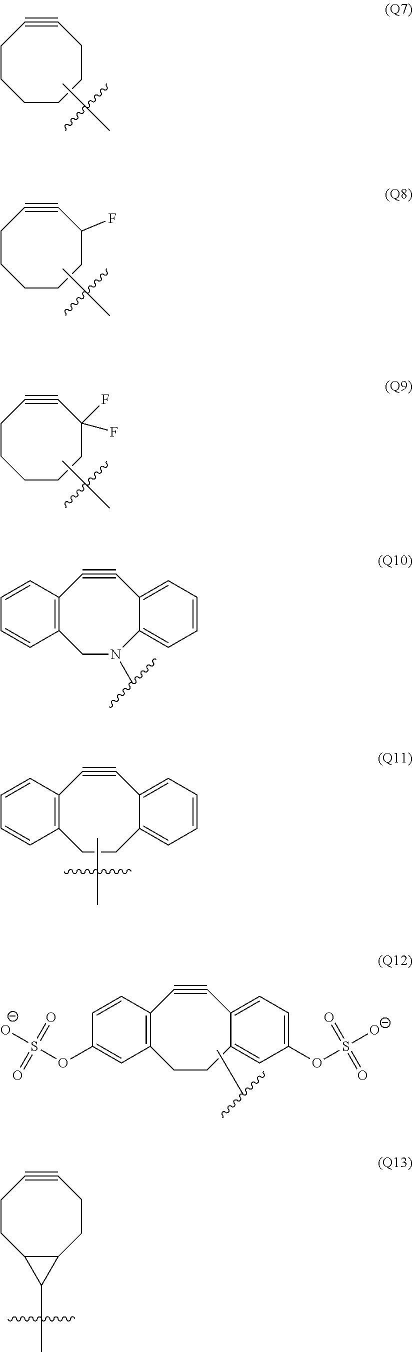

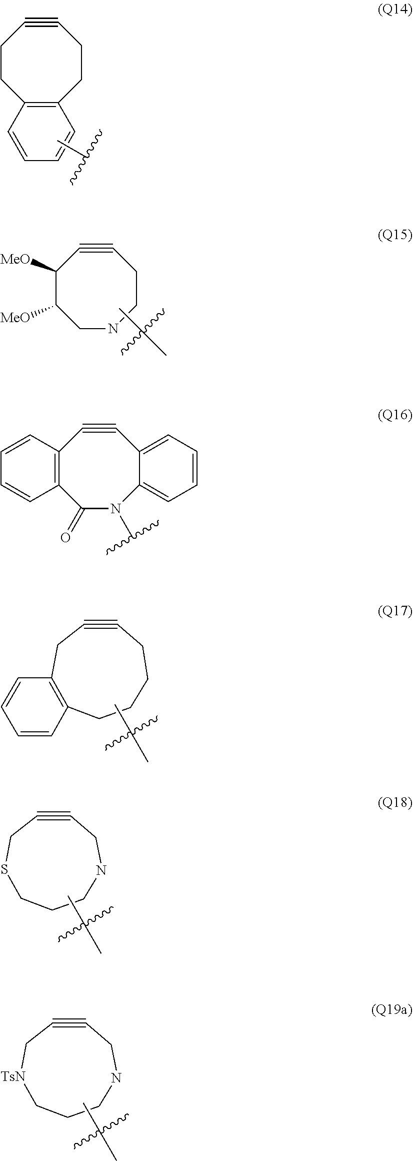

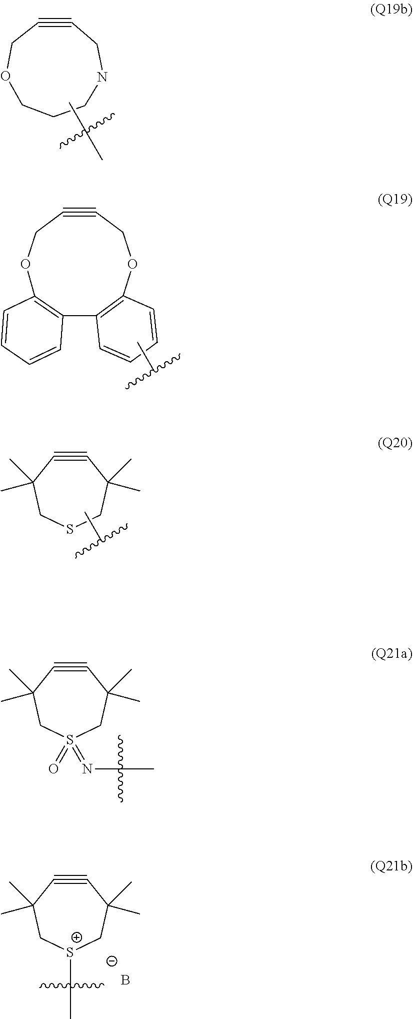

Herein, the connection to L², depicted with the wavy bond, may be to any available carbon or nitrogen atom of Q. B⁽⁻⁾ is an anion, which is preferably selected from OTf, Cl, Br or I, most preferably B⁽⁻⁾ is HOTf. In the conjugation reaction, B⁽⁻⁾ does not need to be a pharmaceutically acceptable anion, since B⁽⁻⁾ will exchange with the anions present in the reaction mixture anyway. In case (Q21b) is used for Q, the negatively charged counter-ion is preferably pharmaceutically acceptable upon isolation of the conjugate according to the invention, such that the conjugate is readily useable as medicament.

In a further preferred embodiment, the click probe Q is selected from the group consisting of (Q22)-(Q36) depicted here below.

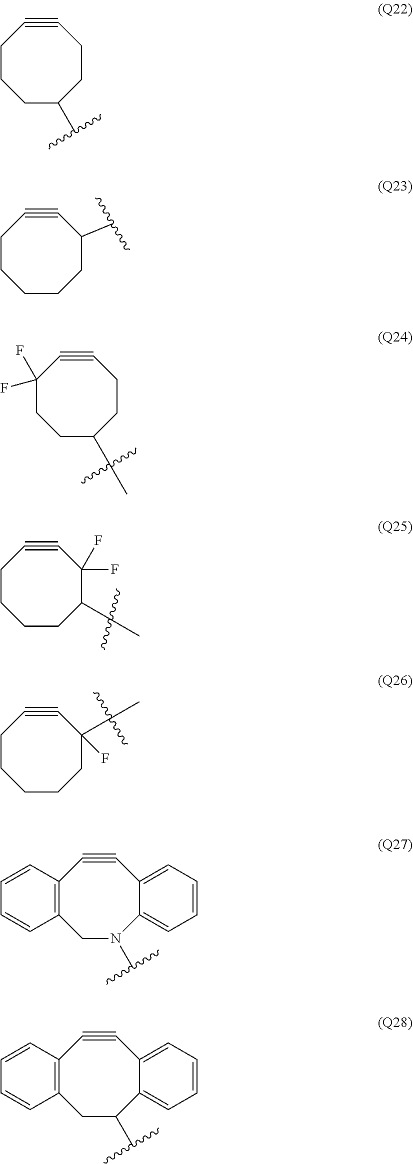

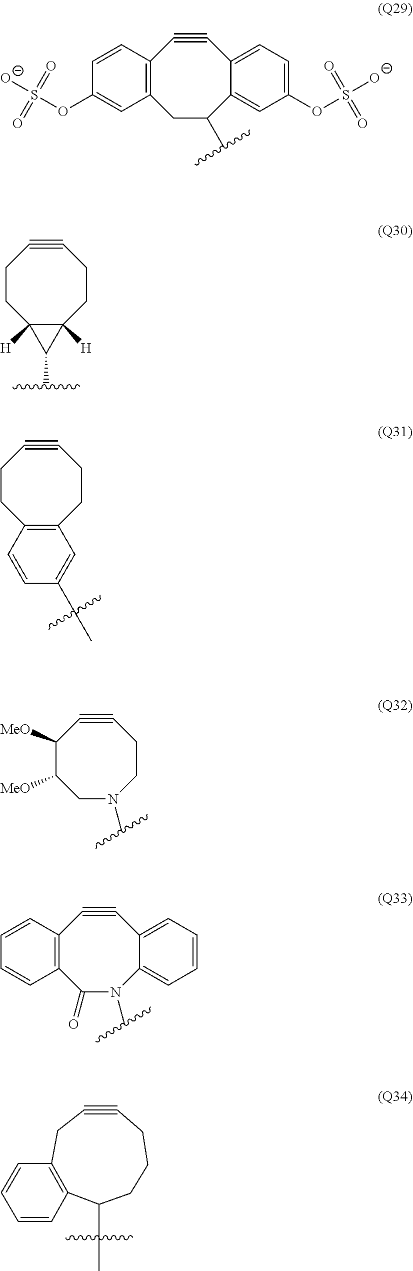 [SMILES: *C1Cc2cc(OS(=O)(=O)[O-])ccc2C#Cc2ccc(OS(=O)(=O)[O-])cc21 ; *[C@H]1[C@H]2CCC#CCC[C@@H]12 ; *c1ccc2c(c1)CCC#CCC2 ; *N1CCC#C[C@H](OC)[C@@H](OC)C1 ; *N1C(=O)c2ccccc2C#Cc2ccccc21 ; *C1CCCC#CCc2ccccc21]

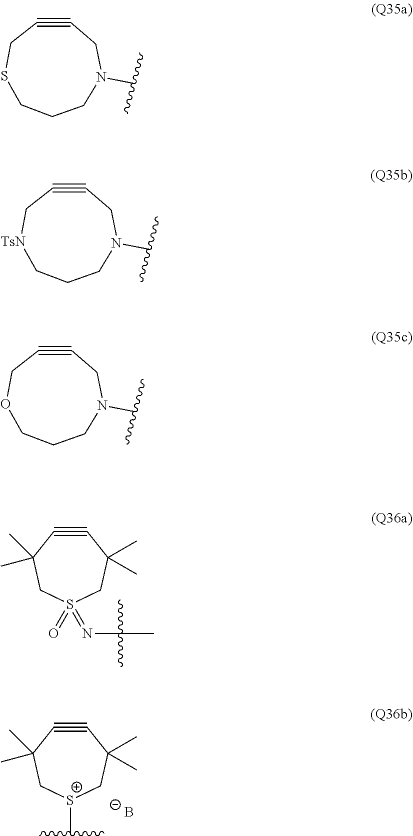

In structure (Q36b), B⁽⁻⁾ is an anion, which is preferably selected from OTf, Cl, Br or I, most preferably B is OTf.

In an especially preferred embodiment, click probe Q comprises an (hetero)cycloalkynyl group and is according to structure (Q37):

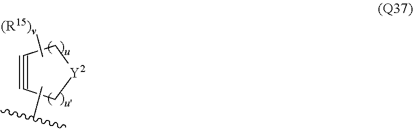

### Herein:

R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S or NR³¹, wherein each R³¹ individually is R¹⁵ or a second occurrence of the exatecan payload connected via a spacer moiety;
u is 0, 1, 2, 3, 4 or 5;
u′ is 0, 1, 2, 3, 4 or 5, wherein u+u′=4, 5, 6, 7 or 8;
v=an integer in the range 8-16.

In a preferred embodiment, u+u′=4, 5 or 6, more preferably u+u′=5. Typically, v=(u+u′)×2 or [(u+u′)×2]-1. In a preferred embodiment, v=8, 9 or 10, more preferably v=9 or 10, most preferably v=10.

In an especially preferred embodiment, click probe Q comprises a cyclooctynyl group and is according to structure (Q38):

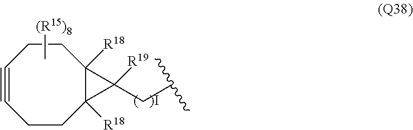

### Herein:

R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of the exatecan payload connected via a spacer moiety; and
I is an integer in the range 0 to 10.

In a preferred embodiment of the reactive group according to structure (Q38), R¹⁵ is independently selected from the group consisting of hydrogen, halogen, —OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the reactive group according to structure (Q38), R¹⁸ is independently selected from the group consisting of hydrogen, C₁-C₆ alkyl groups, most preferably both R¹⁸ are H. In a preferred embodiment of the reactive group according to structure (Q38), R¹⁹ is H. In a preferred embodiment of the reactive group according to structure (Q38), I is 0 or 1, more preferably I is 1. An especially preferred embodiment of the reactive group according to structure (Q38) is the reactive group according to structure (Q30).

In an especially preferred embodiment, click probe Q comprises a cyclooctynyl group and is according to structure (Q39):

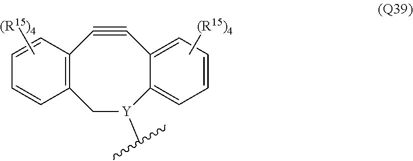

### Herein:

R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

In a preferred embodiment of the reactive group according to structure (Q39), R¹⁵ is independently selected from the group consisting of hydrogen, halogen, —OR¹⁶, —S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃⁽⁻⁾. In a preferred embodiment of the reactive group according to structure (Q39), Y is N or CH, more preferably Y=N.

In an especially preferred embodiment, click probe Q comprises a heterocycloheptynyl group and is according to structure (Q36a):

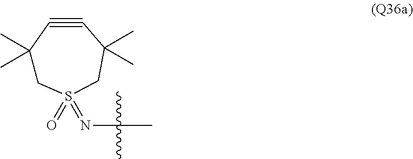

In an alternative preferred embodiment, click probe Q comprises a cyclic alkene moiety. The alkenyl group Q may also be referred to as a (hetero)cycloalkenyl group, i.e. a heterocycloalkenyl group or a cycloalkenyl group, preferably a cycloalkenyl group, wherein the (hetero)cycloalkenyl group is optionally substituted. Preferably, the (hetero)cycloalkenyl group is a (hetero)cyclopropenyl group, a (hetero)cyclobutenyl group, a trans-(hetero)cycloheptenyl group, a trans-(hetero)cyclooctenyl group, a trans-(hetero)cyclononenyl group or a trans-(hetero)cyclodecynyl group, which may all optionally be substituted. Especially preferred are (hetero)cyclopropenyl groups, trans-(hetero)cycloheptenyl group or trans-(hetero)cyclooctenyl groups, wherein the (hetero)cyclopropenyl group, the trans-(hetero)cycloheptenyl group or the trans-(hetero)cyclooctynyl group is optionally substituted. Preferably, Q comprises a cyclopropenyl moiety according to structure (Q40), a trans-(hetero)cycloheptenyl moiety according to structure (Q41) or a trans-(hetero)cyclooctenyl moiety according to structure (Q42). In a further preferred embodiment, the cyclopropenyl group is according to structure (Q43). In another preferred embodiment, the trans-(hetero)cycloheptene group is according to structure (Q44) or (Q45). In another preferred embodiment, the trans-(hetero)cyclooctene group is according to structure (Q46), (Q47), (Q48), (Q49) or (Q50).

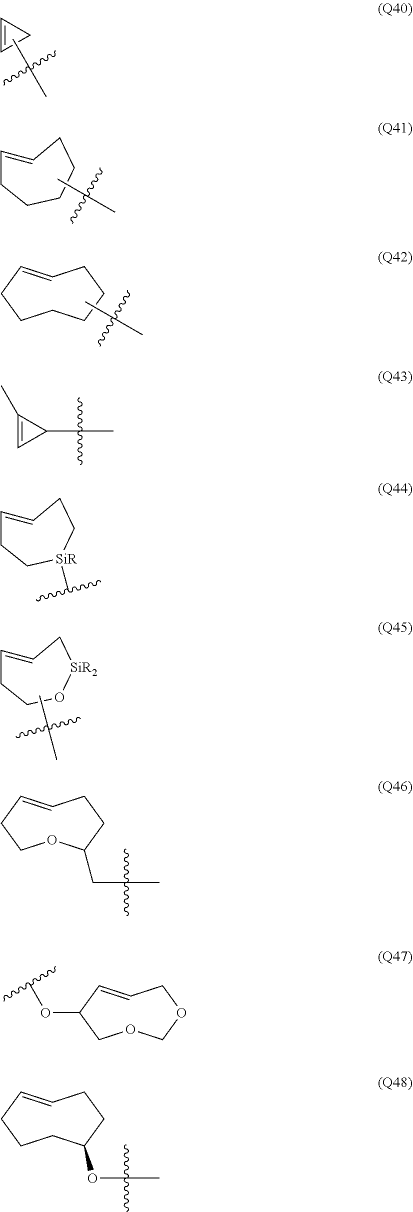

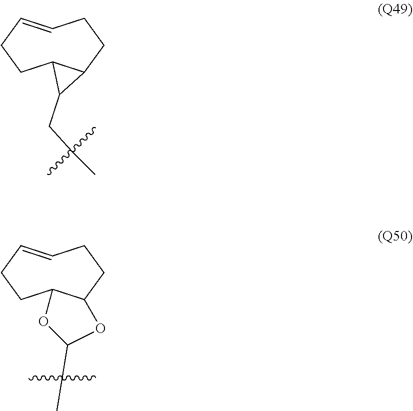 [SMILES: *CC1C2CC/C=C/CCC12 ; *C1OC2CC/C=C/CCC2O1]

Herein, the R group(s) on Si in (Q44) and (Q45) are typically alkyl or aryl, preferably C₁-C₆ alkyl.

### Thiol Ligation

Thiol ligation for the preparation of antibody-drug conjugates is well-known in the art, and may also be referred to as alkylation of a thiol group. It typically takes the form of a nucleophilic reaction, such as a nucleophilic substitution or a Michael reaction. A preferred Michael reaction is the maleimide-thiol reaction, which is widely employed in bioconjugation. Thus, in a preferred embodiment, Q is reactive in a nucleophilic reaction, preferably in a nucleophilic substitution or a Michael reaction. Herein, it is preferred that Q comprises a maleimide moiety, a haloacetamide moiety, an allenamide moiety, a phosphonamidite moiety, a cyanoethynyl moiety, a vinylsulfone, a vinylpyridine moiety or a methylsulfonylphenyloxadiazole moiety, most preferably a maleimide moiety. Especially preferred options for Q are depicted in FIG. 1B.

### Thiol-Reactive Probe Q

Thiol-reactive probe Q is used in the conjugation reaction to connect the linker-drug construct to the antibody of structure AB-((L¹)_{w}-F)ₓ. Q is reactive towards thiol or precursor thereof F in a thiol ligation. Such probes are known in the art and may be selected from the group consisting of a maleimide moiety, a haloacetamide moiety, an allenamide moiety, a phosphonamidite moiety, a cyanoethynyl moiety, a vinylsulfone, a vinylpyridine moiety or a methylsulfonylphenyloxadiazole moiety. Most preferably, Q comprises or is a maleimide moiety.

In a further preferred embodiment, probe Q is selected from the group consisting of (Q51)-(Q65) depicted here below.

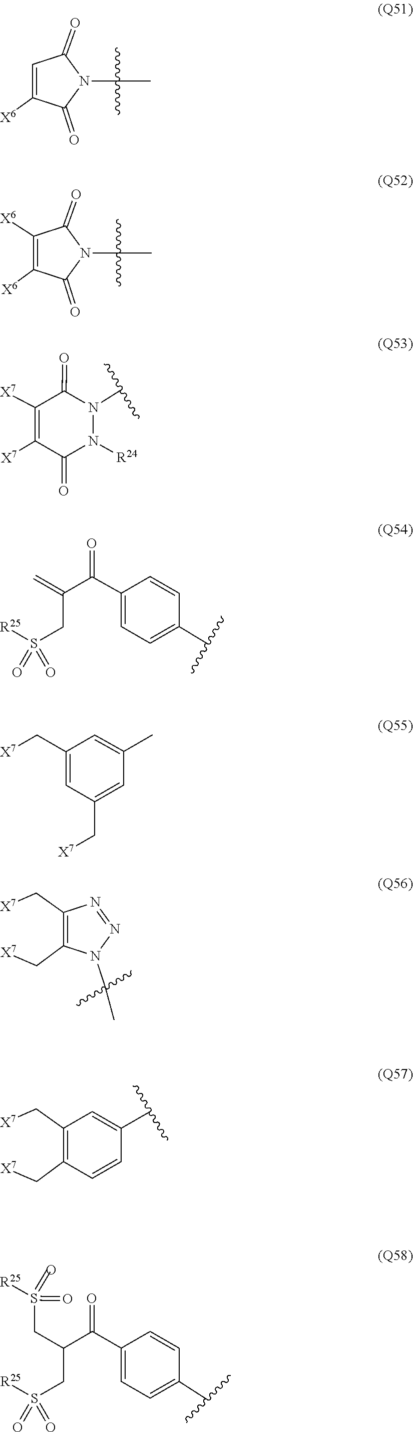

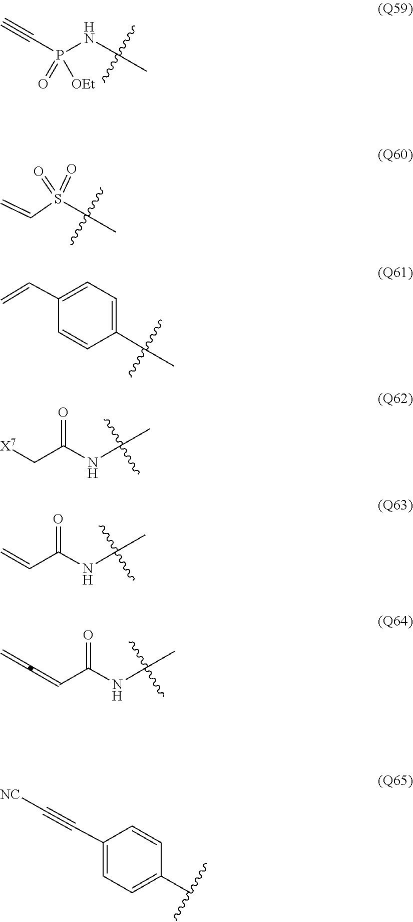

wherein:
X⁶ is H, halogen, PhS, MeS, preferably a halogen, such as Cl, Br, I;
X⁷ is halogen, PhS, MeS, preferably a halogen, such as Cl, Br, I;
R²⁴ is H or C₁₋₁₂ alkyl, preferably H or C₁₋₆ alkyl;
R²⁵ is H, C₁₋₁₂ alkyl, C₁₋₁₂ aryl, C₁₋₁₂ alkaryl or C₁₋₁₂ aralkyl, preferably H or para-methylphenyl;
wherein the aromatic ring of (Q55) and (Q57) may optionally be a heteroaromatic ring, such as a phenyl or pyridine ring.

In a preferred embodiment of thiol-reactive probe (Q51), the probe Q is selected from the group consisting of (Q66)-(Q68) depicted here below.

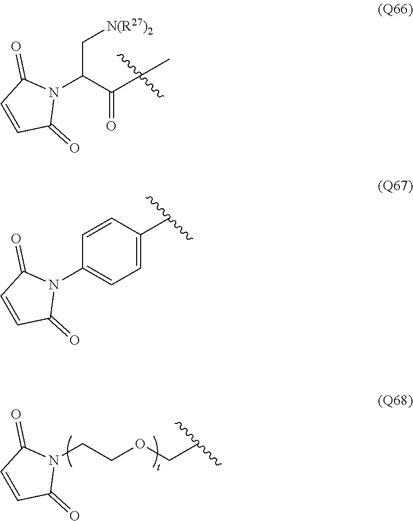

wherein:
R²⁷ is C₁₋₁₂ alkyl, C₁₋₁₂ aryl, C₁₋₁₂ alkaryl or C₁₋₁₂ aralkyl;
t is an integer in the range of 0-15, preferably 1-10.

### Preferred Linker-Drug Constructs

In an especially preferred embodiment, the linker-drug construct according to the invention has structure (5c):

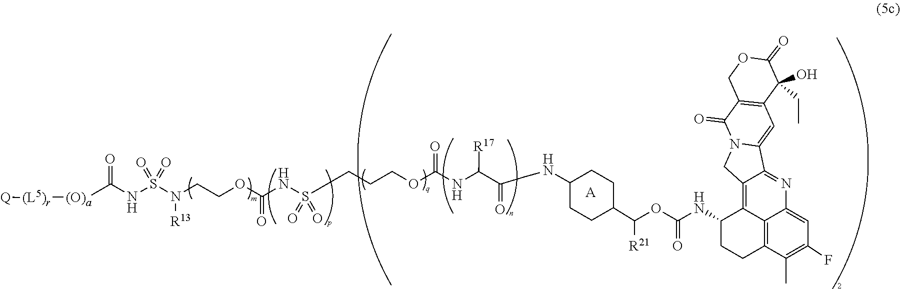

wherein each of Q, m, p, q, R¹⁷, A and R²¹ are as defined above.

Most preferably, the linker-drug construct according to the invention has structure (5a), wherein
Q is a connecting group according to structure (Q38), preferably wherein R¹⁵, R¹⁸ and R¹⁹ are all H and I is 1;
each R¹⁷ is individually CH₃ or CH₂CH₂CH₂NHC(O)NH₂, preferably each R¹⁷ is CH₃;
m is 2 or 4, preferably m=2
p is 0 or 1, preferably p=1
each q is 2 or 4, preferably each q=2
each A is 1,4-phenyl;
each R²¹ is H.

In an especially preferred embodiment, the linker-drug construct according to the invention has structure (5a):

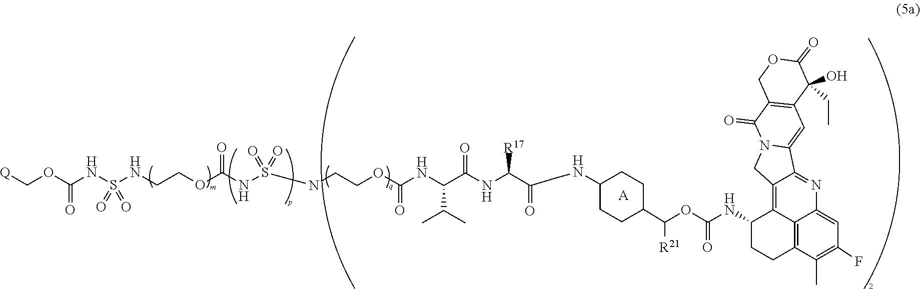

wherein each of Q, m, p, q, R¹⁷, A and R²¹ are as defined above.

Most preferably, the linker-drug construct according to the invention has structure (5a), wherein
Q is a connecting group according to structure (Q38), preferably wherein R¹⁵, R¹⁸ and R¹⁹ are all H and I is 1;
each R¹⁷ is individually CH₃ or CH₂CH₂CH₂NHC(O)NH₂, preferably each R¹⁷ is CH₃;
m is 2 or 4, preferably m=2
p is 0 or 1, preferably p=1
each q is 2 or 4, preferably each q=2
each A is 1,4-phenyl;
each R²¹ is H.

In an especially preferred embodiment, the linker-drug construct according to the invention has structure (5b):

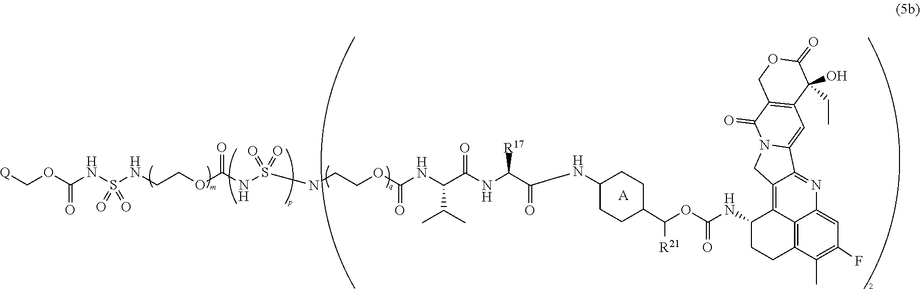

wherein each of Q, m, p, q, R¹⁷, A and R²¹ are as defined above.

Most preferably, the linker-drug construct according to the invention has structure (5a), wherein
Q is a connecting group according to structure (Q1);
each R¹⁷ is individually CH₃ or CH₂CH₂CH₂NHC(O)NH₂, preferably each R¹⁷ is CH₃;
m is 2 or 4, preferably m=2
p is 0 or 1, preferably p=1
each q is 2 or 4, preferably each q=2
each A is 1,4-phenyl;
each R²¹ is H.

In an especially preferred embodiment, the linker-drug construct according to the invention has structure (5d):

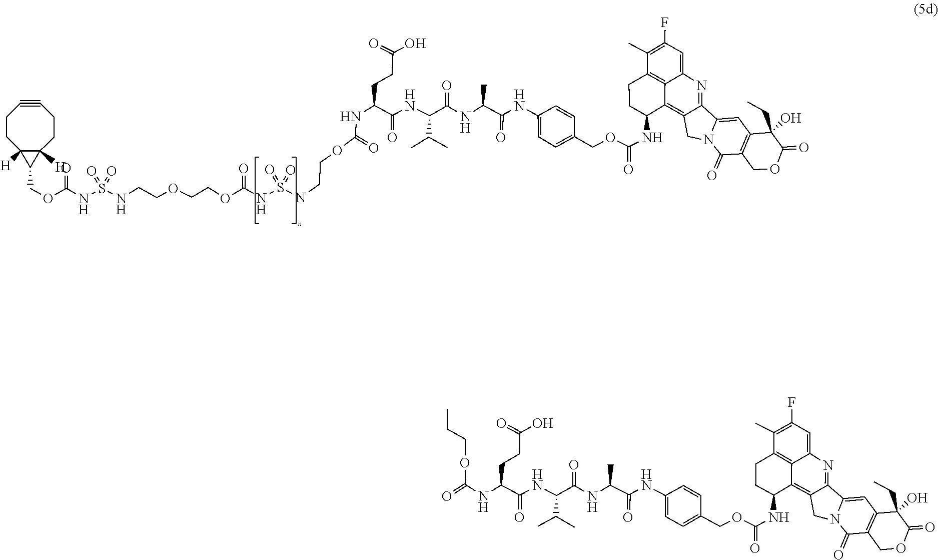

wherein n=0 or 1.

Structure (5d) corresponds to compounds (31a), with n=0, and (32a), with n=1, in the examples.

### Reactive Group F

Reactive group F is click probe or a thiol or precursor thereof. Thus, in a first preferred embodiment, F is a click probe. Click probe F is used in the conjugation reaction to connect the linker-drug construct to the modified antibody. F is reactive towards click probe Q in a metal-free click reaction. Such click probes are known in the art and include cyclic alkene, cyclic alkyne, azide, tetrazine, triazine, nitrone, nitrile oxide, nitrile imine, diazo compound, ortho-quinone, dioxothiophene and sydnone.

Preferably, F is reactive towards a cyclic alkene, cyclic alkyne, and is typically selected from the group consisting of azide, tetrazine, triazine, nitrone, nitrile oxide, nitrile imine, diazo compound, ortho-quinone, dioxothiophene and sydnone. Preferred structures for the reactive group are structures (F1)-(F10) depicted here below. PGP-88 C₃

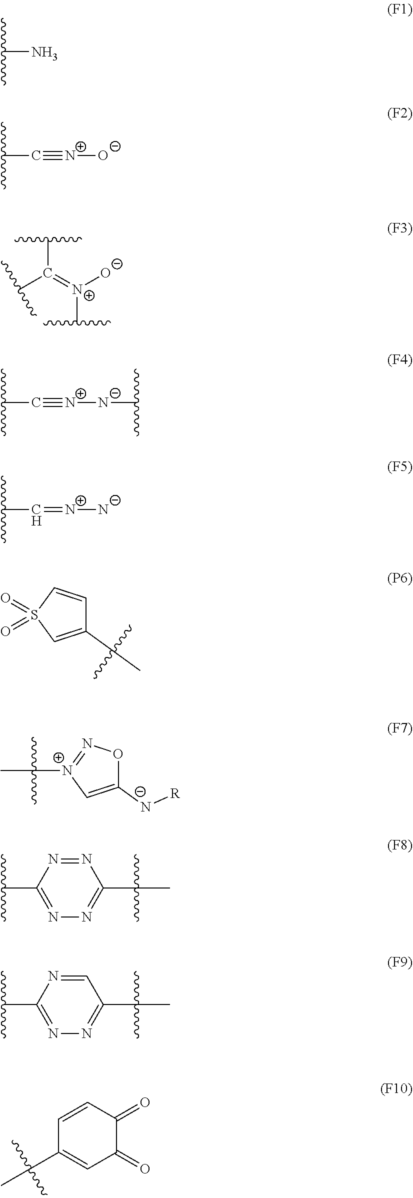

Herein, the wavy bond represents the connection to the antibody. For (F3), (F4), (F8) and (F9), the antibody can be connected to any one of the wavy bonds. The other wavy bond may then be connected to an R group selected from hydrogen, C₁-C₂₄ alkyl groups, C₂-C₂₄ acyl groups, C₃-C₂₄ cycloalkyl groups, C₂-C₂₄ (hetero)aryl groups, C₃-C₂₄ alkyl(hetero)aryl groups, C₃-C₂₄ (hetero)arylalkyl groups and C₁-C₂₄ sulfonyl groups, each of which (except hydrogen) may optionally be substituted and optionally interrupted by one or more heteroatoms selected from O, S and NR³² wherein R³² is independently selected from the group consisting of hydrogen and C₁-C₄ alkyl groups. The skilled person understands which R groups may be applied for each of the click probes F. For example, the R group connected to the nitrogen atom of (F3) may be selected from alkyl and aryl, and the R group connected to the carbon atom of (F3) may be selected from hydrogen, alkyl, aryl, acyl and sulfonyl. Preferably, click probe F is selected from azides or tetrazines. Most preferably, click probe F is an azide.

In a second preferred embodiment, F is a thiol or precursor thereof. Thiol or precursor thereof F is used in the conjugation reaction to connect the linker-drug construct to the modified antibody. F is reactive towards thiol-reactive probe Q in a thiol ligation. Thiol precursors in the context of bioconjugation are known in the art, and include disulfides, which may be naturally occurring disulfide bridged present in the antibody or synthetically introduced disulfides, which are reduced as known in the art. Preferably, F is a thiol group.

### The Linker-Drug Construct

In a third aspect, the invention concerns a linker-drug construct according to structure (5):

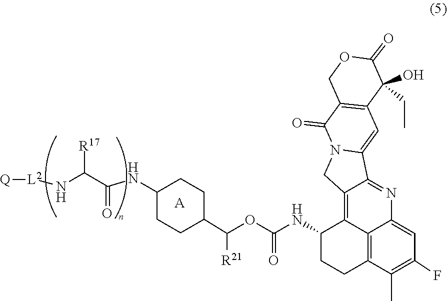

wherein:
L² is a linker;
Q is a click probe capable of performing a metal-free click reaction or a thiol-reactive probe;
each R¹⁷ is individually an amino acid side chain;
n is an integer in the range of 1-5;
A is a 5- or 6-membered aromatic or heteroaromatic ring;
x is an integer in the range of 1-8;
R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which are optionally substituted and optionally interrupted by one or more heteroatoms selected from O, S and NR²³ wherein R²³ is independently selected from the group consisting of hydrogen and C₁-C₄ alkyl groups.

The definitions and preferred embodiments of the linker-drug construct and L², Q, R¹⁷, n, A, x and R²¹ provided in the context of the first and second aspect of the invention equally apply to the third aspect of the invention. The linker-drug construct according to this aspect are ideally suitable as intermediate in the preparation of the antibody-drug conjugates according to the present invention. The invention thus also concerns the use of the linker-drug construct according to the invention in a metal-free click conjugation reaction or a thiol ligation conjugation reaction with a modified antibody of structure AB-((L¹)_{w}-F)ₓ.

### Application

The conjugates according to the invention are especially suitable in the treatment of cancer. The invention thus further concerns the use of the conjugate according to the invention in medicine. In a further aspect, the invention also concerns a method of treating a subject in need thereof, comprising administering the conjugate according to the invention to the subject. The method according to this aspect can also be worded as the conjugate according to the invention for use in treatment, in particular for use in the treatment of a subject in need thereof. The method according to this aspect can also be worded as use of the conjugate according to the invention for the manufacture of a medicament. Herein, administration typically occurs with a therapeutically effective amount of the conjugate according to the invention.

The invention further concerns a method for the treatment of a specific disease in a subject in need thereof, comprising the administration of the conjugate according to the invention as defined above. The specific disease may be selected from cancer and an autoimmune disease, preferably the disease is cancer. The subject in need thereof is typically a cancer patient. The use of antibody-drug conjugates is well-known in such treatments, especially in the field of cancer treatment, and the conjugates according to the invention are especially suited in this respect. In the method according to this aspect, the conjugate is typically administered in a therapeutically effective amount. The present aspect of the invention can also be worded as a conjugate according to the invention for use in the treatment of a specific disease in a subject in need thereof, preferably for the treatment of cancer. In other words, this aspect concerns the use of a conjugate according to the invention for the preparation of a medicament or pharmaceutical composition for use in the treatment of a specific disease in a subject in need thereof, preferably for use in the treatment of cancer. In one embodiment, the cancer is a HER2-positive cancer, such as HER2-positive breast cancer, HER2-positive stomach cancer, HER2-positive colon cancer, HER2-positive lung cancer, HER2-positive pancreatic cancer, HER2-positive urothelial cancer, HER2-positive brain cancer, HER2-positive ovarian cancer. Thus, in one embodiment, the patient is HER2-positive.

Administration in the context of the present invention refers to systemic administration. Hence, in one embodiment, the methods defined herein are for systemic administration of the conjugate. In view of the specificity of the conjugates, they can be systemically administered, and yet exert their activity in or near the tissue of interest (e.g. a tumour). Systemic administration has a great advantage over local administration, as the drug may also reach tumour metastasis not detectable with imaging techniques and it may be applicable to hematological tumours.

The invention further concerns a pharmaceutical composition comprising the antibody-payload conjugate according to the invention and a pharmaceutically acceptable carrier.

### EXAMPLES

The invention is illustrated by the following examples.

### General Procedure for Analytical RP-HPLC (DTT Reduction)

Prior to RP-HPLC analysis, IgG (10 μL, 1 mg/mL in PBS pH 7.4) was added to 12.5 mM DTT, 100 mM TrisHCl pH 8.0 (40 μL) and incubated for 15 minutes at 37° C. The reaction was quenched by adding 49% acetonitrile, 49% water, 2% formic acid (50 μL). RP-HPLC analysis was performed on an Agilent 1100 series (Hewlett Packard). The sample (10 μL) was injected with 0.5 mL/min onto Bioresolve RP mAb 2.1*150 mm 2.7 μm (Waters) with a column temperature of 70° C. A linear gradient was applied in 16.8 minutes from 30 to 54% acetonitrile in 0.1% TFA and water.

### General Procedure for Analytical RP-UPLC

Prior to RP-UPLC analysis, IgG (10 μL, 1 mg/mL in PBS pH 7.4) was added to 12.5 mM DTT, 100 mM TrisHCl pH 8.0 (40 μL) and incubated for 15 minutes at 37° C. The reaction was quenched by adding 49% acetonitrile, 49% water, 2% formic acid (50 μL). RP-UPLC analysis was performed on a Waters Acquity UPLC-SQD. The sample (5 μL) was injected with 0.4 mL/min onto Bioresolve RP mAb 2.1*150 mm 2.7 μm (Waters) with a column temperature of 70° C. A linear gradient was applied in 9 minutes from 30 to 54% acetonitrile in 0.1% TFA and water.

### General Procedure for Analytical SEC

HPLC-SEC analysis was performed on an Agilent 1100 series (Hewlett Packard) using an Xbridge BEH200A (3.5 μM, 7.8×300 mm, PN 186007640 Waters) column. The sample was diluted to 1 mg/mL in PBS and measured with 0.86 mL/min isocratic method (0.1 M sodium phosphate buffer pH 6.9 (NaHPO₄/Na₂PO₄) containing 10% isopropanol) for 16 minutes.

### General Procedure for Analytical HPLC-MS (IdeS Digestion)

Prior to mass spectral analysis, IgG was treated with IdeS, which allows analysis of the Fc/2 fragment. For analysis of the Fc/2 fragment, a solution of 20 μg (modified) IgG was incubated for 1 hour at 37° C. with IdeS/Fabricator™ (1.25 U/μL) in PBS pH 6.6 in a total volume of 10 μL. Samples were diluted to 80 μL followed by analysis electrospray ionization time-of-flight (ESI-TOF) on a JEOL AccuTOF. Deconvoluted spectra were obtained using Magtran software.

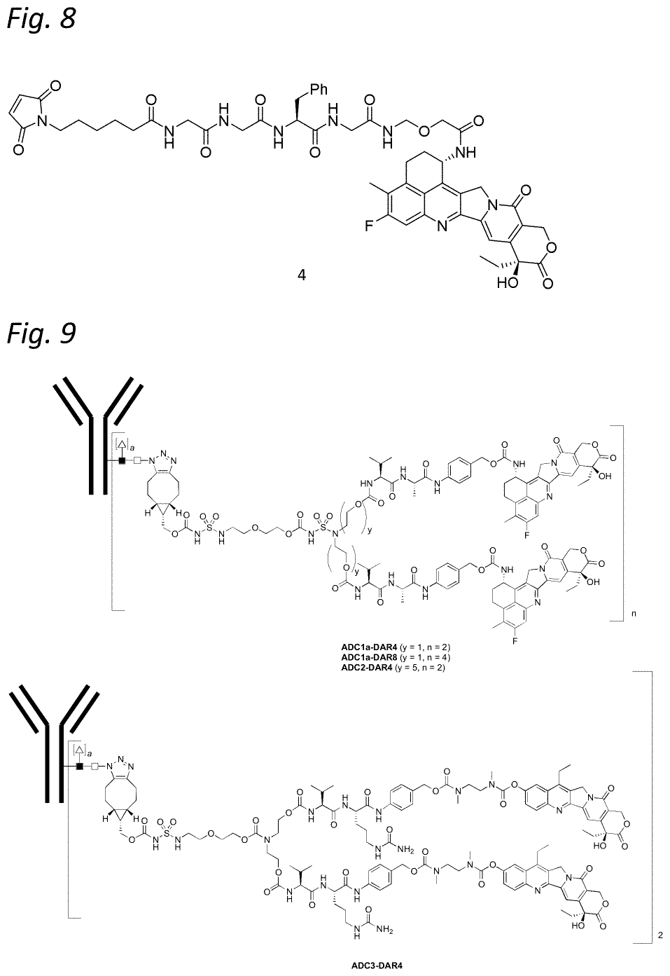

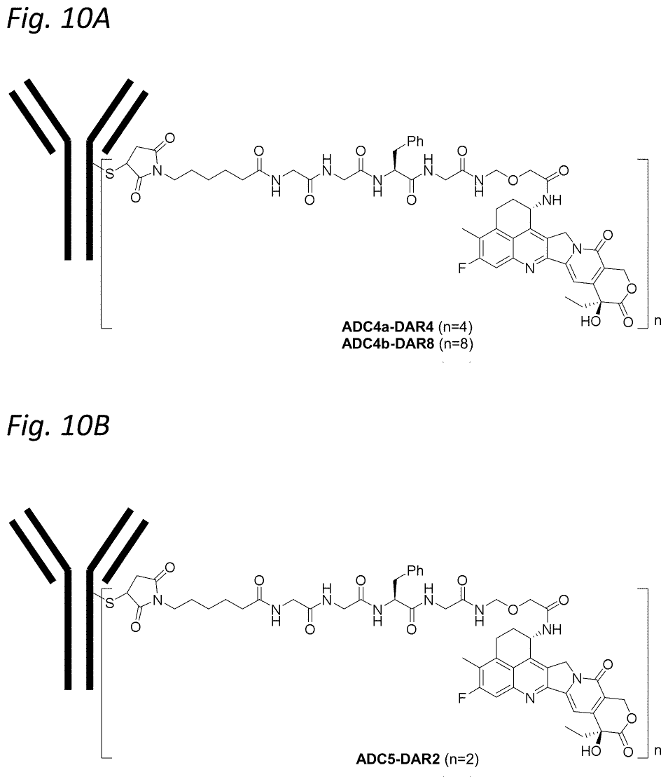

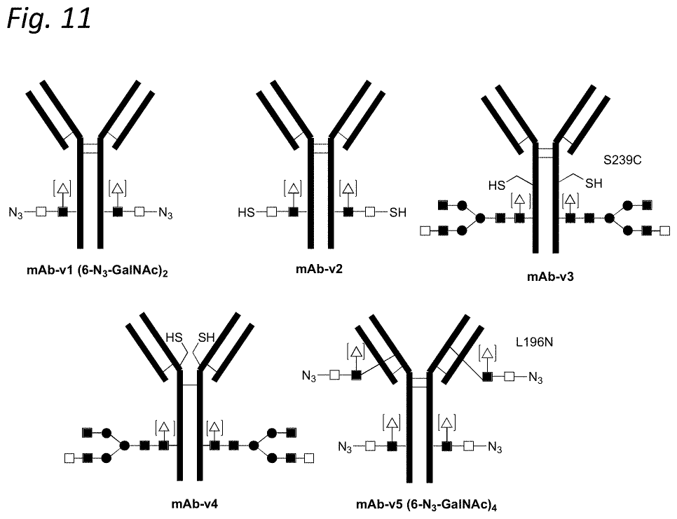

Compound 3 (see FIG. 7) was prepared according to WO 2020/094670 (linker-drug compound 42). Compound 4 (deruxtecan, see FIG. 8) was obtained from MedChemExpress. Structures of various antibody formats for metal-free click conjugation (v1 and v5) or thiol ligation (v2, v3, v4) are depicted in FIG. 11.

### Example 1. Preparation of Compound 6

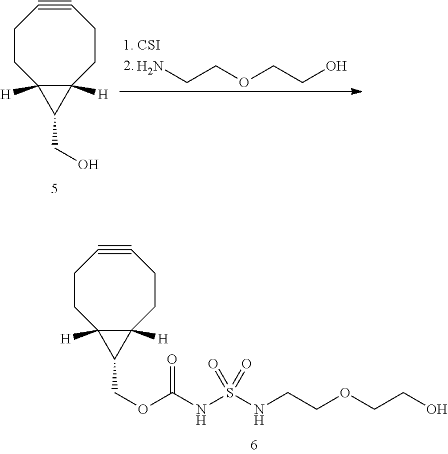 [SMILES: OC[C@H]1[C@H]2CCC#CCC[C@@H]12 ; NCCOCCO ; O=C(NS(=O)(=O)NCCOCCO)OC[C@@H]1[C@@H]2CCC#CCC[C@@H]21]

To a solution of BCN—OH (5, 1.5 g, 10 mmol) in DCM (150 mL), under a N₂ atmosphere, was added CSI (0.87 mL, 1.4 g, 10 mmol), Et₃N (2.8 mL, 2.0 g, 20 mmol) and 2-(2-aminoethoxy)ethanol (1.2 mL, 1.26 g, 12 mmol). The mixture was stirred for 10 min and quenched through addition of aqueous NH₄Cl (sat., 150 mL). After separation, the aqueous layer was extracted with DCM (150 mL). The combined organic layers were dried (Na₂SO₄) and concentrated. The residue was purified with column chromatography. Product 6 was obtained as slightly yellow thick oil (2.06 g, 5.72 mmol, 57%). 1H NMR (400 MHz, CDCl₃) δ (ppm) 6.0 (bs, 1H), 4.28 (d, J=8.2 Hz, 2H), 3.78-3.73 (m, 2H), 3.66-3.61 (m, 2H), 3.61-3.55 (m, 2H), 3.34 (t, J=4.9 Hz, 2H), 2.37-2.15 (m, 6H), 1.64-1.48 (m, 2H), 1.40 (quintet, J=8.7 Hz, 1H), 1.05-0.92 (m, 2H).

### Example 2. Preparation of Compound 7

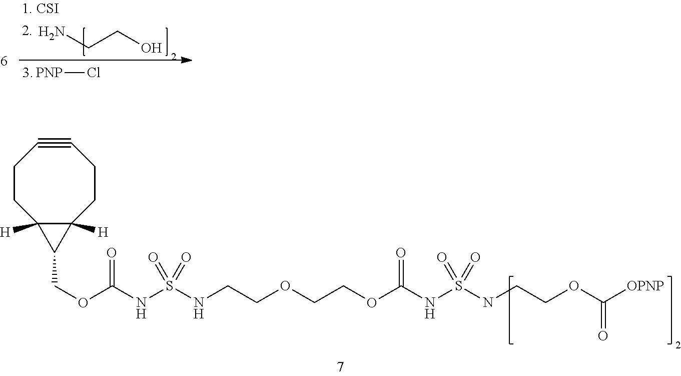

To a stirring solution of 6 (47 mg, 0.13 mmol) in DCM (10 mL) was added CSI (11 μL, 18 mg, 0.13 mmol). After 30 min, Et₃N (91 μL, 66 mg, 0.65 mmol) and a solution of diethanolamine (16 mg, 0.16 mmol) in DMF (0.5 mL) were added. After 30 minutes p-nitrophenyl chloroformate (52 mg, 0.26 mmol) and Et₃N (54 μL, 39 mg, 0.39 mmol) were added. After an additional 4.5 h, the reaction mixture was concentrated and the residue was purified by gradient column chromatography (33→66% EtOAc/heptane (1% AcOH)) to afford 7 as colourless oil (88 mg, 0.098 mmol, 75%). ¹H NMR (400 MHz, CDCl₃) δ (ppm) 8.28-8.23 (m, 4H), 7.42-7.35 (m, 4H), 4.52 (t, J=5.4 Hz, 4H), 4.30 (d, J=8.3 Hz, 2H), 4.27-4.22 (m, 2H), 3.86 (t, J=5.3 Hz, 4H), 3.69-3.65 (m, 2H), 3.64-3.59 (m, 2H), 3.30-3.22 (m, 2H), 2.34-2.14 (m, 6H), 1.62-1.46 (m, 2H), 1.38 (quintet, J=8.7 Hz, 1H), 1.04-0.92 (m, 2H).

### Example 3. Preparation of Compounds 9a and 9b

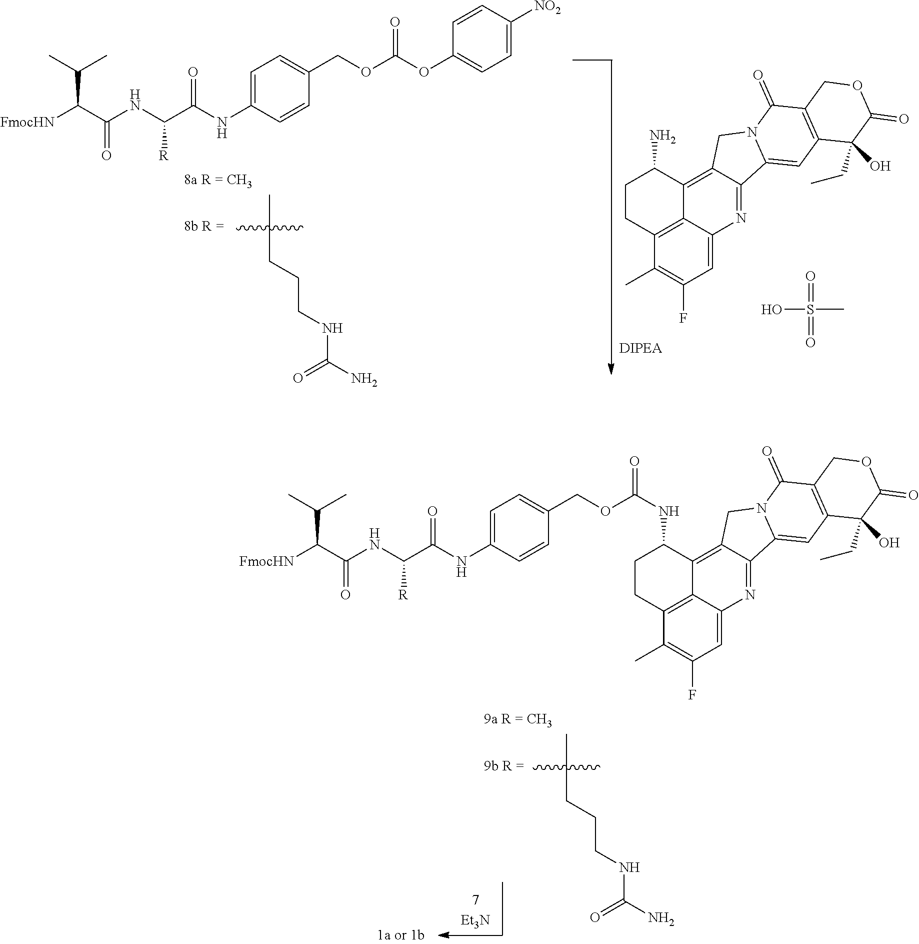

Compound 8a (163 mg, 240 μmol) was added to a mixture of exatecan mesylate (125 mg, 235 μmol) and DIPEA (61 mg, 82 μL, 0.47 mmol) in dry DMF (0.9 mL). After 20 h, the reaction mixture was diluted to 9 mL DCM and purified by gradient column chromatography (0→40% MeOH/DCM) to afford 9a (155 mg, 159 μmol, 68%). LCMS (ESI+) calculated for C₅₅H₅₄FN₆O₁₀⁺ (M+H)⁺ 977.39, found 977.72. In addition to 9a, free base of exatecan (82.4 mg, 189 μmol, 20%) was recovered. LCMS (ESI+) calculated for C₂₄H₂₃FN₃O₄⁺ (M+H)⁺ 436.46, found 436.54.

Compound 8b (29.1 mg, 38 μmol) was added to a mixture of exatecan mesylate (19.8 mg, 37.2 μmol) and DIPEA (9.6 mg, 13 μL, 74.5 μmol) in dry DMF (150 μL). After 5 h, the reaction mixture was diluted to 3 mL DCM and purified by automated gradient column chromatography over silica gel (0→10% MeOH/DCM) to afford 9b (28.2 mg, 26.5 μmol, 74%) as a pale-yellow solid. LCMS (ESI+) calculated for C₅₈H₆₀FN₈O₁₁⁺ (M+H)⁺ 1063.44, found 1063.72.

### Example 4. Preparation of Compounds 1a and 1b (for Structures See FIG. 7)

To a solution of compound 9a (155 mg, 159 μmol) in DMF (1.6 mL) were added Et₃N (73 mg, 101 μL, 0.72 mmol) and a solution of compound 7 (65 mg, 72 μmol) in DMF (1.4 mL). The reaction mixture was stirred for 18 h, diluted with DCM (20 mL) and purified by gradient column chromatography (0→40% MeOH/DCM) to afford 1a as a pale-yellow solid (94 mg, 44 μmol, 28%). LCMS (ESI+) calculated for C₁₀₂H₁₁₈F₂N₁₆O₂₉S₂²⁺ (M/2+H)⁺ 1066.88, found 1067.12.

To a solution of compound 7 (3.5 mg, 3.9 μmol) in DMF (78 μL) were added a solution of compound 9b (8.2 mg, 9.7 μmol) in DMF (97 μL) and Et₃N (2.4 mg, 3.3 μL, 23.4 μmol). The reaction mixture was left for 20 h. The reaction mixture was diluted with DCM (2 mL) and purified by automated gradient column chromatography over silica gel (0→30% MeOH/DCM). Part of the obtained material was re-purified by preparative RP-HPLC (XBridge prep C18 5 um OBD, 30×100 mm, 30→90% MeCN/H₂O+1% AcOH) to afford 1b as a white solid (1.0 mg, 0.43 μmol, 11%). LCMS (ESI+) calculated for C₁₀₈H₁₃₀F₂N₂₀O₃₁S₂²+(M/2+H)⁺ 1152.93, found 1152.58.

### Example 5. Preparation of Compounds 10a and 10b

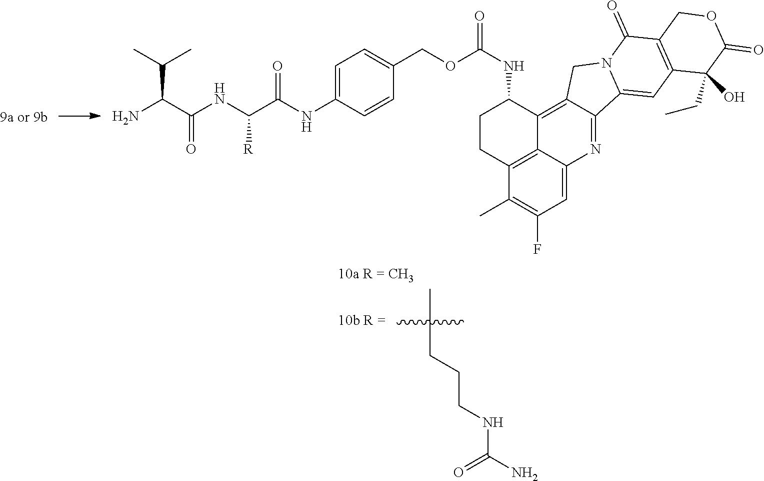

Compound 9a (32 mg, 33 μmol) was dissolved in DMF (1 mL) and piperidine (28 mg, 33 μL, 0.33 mmol) was added. The mixture was stirred for 5.5 h, concentrated, taken up in DCM (2 mL) and purified by gradient column chromatography (0→30% MeOH/DCM (1% AcOH)) to afford 10a (14.8 mg, 19.6 μmol, 59%). LCMS (ESI+) calculated for C₄₀H₄₄FN₆O₈ (M+H)⁺ 755.32, found 755.46.

To a solution of compound 9b (28.2 mg, 26.5 μmol) in DMF (200 μL) was added piperidine (22.6 mg, 26.2 μL, 265 μmol). The reaction mixture was left for 5.5 h. The reaction mixture was diluted with DCM (2 mL) and purified by gradient column chromatography (0→30% MeOH/DCM) to afford 10b (16.3 mg, 19.4 μmol, 73%) as a pale yellow solid. LCMS (ESI+) calculated for C₄₃H₅₀FN₈O₉ (M+H)⁺ 841.37, found 841.53.

### Example 6. Preparation of Compound 2a

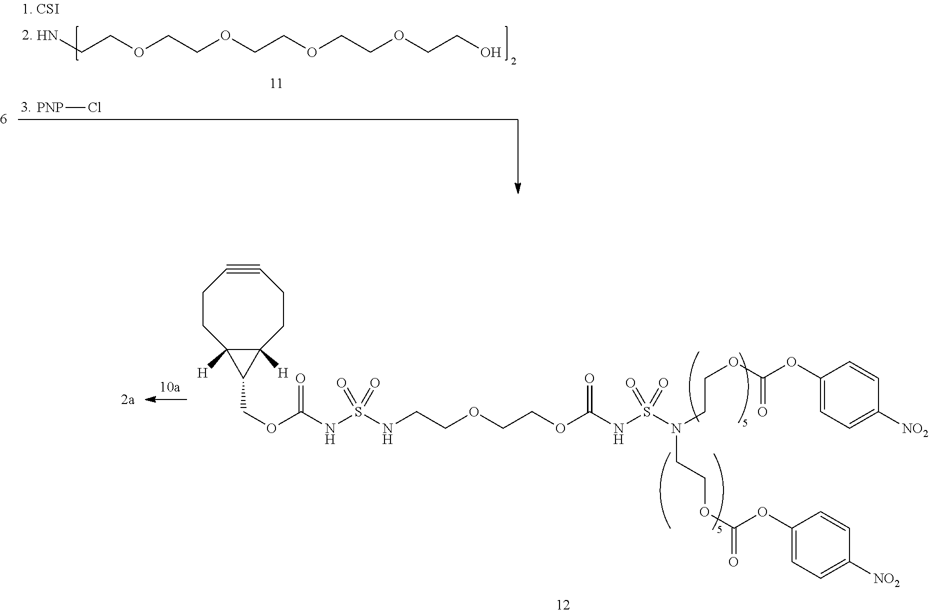

To a stirring solution of 6 (172 mg, 0.48 mmol) in DCM (20 mL) was added CSI (42 μL, 67 mg, 0.48 mmol). After 20 min, Et₃N (331 μL, 240 mg, 2.38 mmol) and a solution of 11 (250 mg, 0.55 mmol) in DMF (1 mL) were added. After 60 min, p-nitrophenyl chloroformate (242 mg, 1.20 mmol) and Et₃N (643 μL, 467 mg, 1.43 mmol) were added. After 16 h, additional p-nitrophenyl chloroformate (50 mg, 0.25 mmol) and stirring was continued for 4 h. The reaction mixture was concentrated and the residue was purified by gradient column chromatography (20→100% EtOAc/heptane (1% AcOH)) to afford 12 as a colorless oil (318 mg, 0.25 mmol, 53%). LCMS (ESI+) calculated for C₅₀H₇₃N₆O₂₇S₂⁺ (M+H⁺) 1253.40, found 1253.49.

To a solution of compound 12 (10.0 mg, 8.0 μmol) in DMF (200 μL) were added a solution of compound 10 (14.8 mg, 19.6 μmol) in DMF (200 μL) and Et₃N (4.0 mg, 5.6 μL, 40 μmol). The reaction mixture was left for 64 h. The reaction mixture was purified by preparative HPLC (XBridge prep C18 5 um OBD, 30×100 mm, 30→100% CH₃CN/H₂O (containing 1% AcOH)) to afford 2a as a pale yellow film (4.3 mg, 1.73 μmol, 22%). LCMS (ESI+) calculated for C₁₁₈H₁₅₀F₂N₁₆O₃₇S₂²⁺. (M/2+H)⁺ 1242.99, found 1243.13.

### Example 7. Preparation of Compound 14

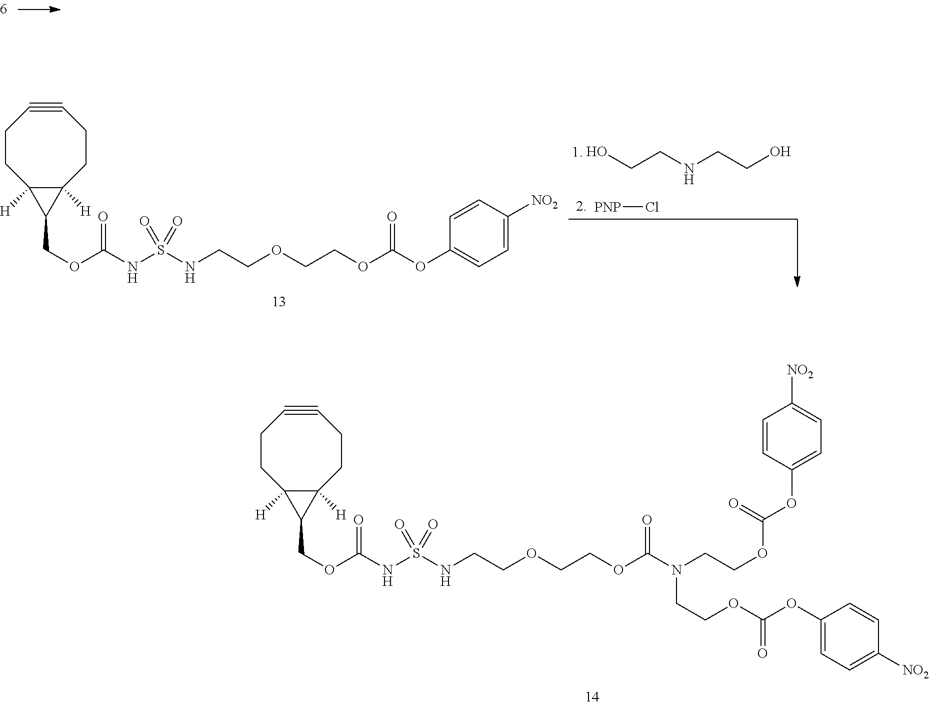

Preparation of 13: To a solution of 6 (3.62 g, 10 mmol) in DCM (200 mL) were added 4-nitrophenyl chloroformate (2.02 g, 10 mmol) and Et₃N (4.2 mL, 3.04 g, 30 mmol). After stirring for 1.5 h at ambient temperature, the reaction mixture was concentrated and purified by automated gradient column chromatography over silica gel (20%→70% EtOAc/heptane+1% AcOH) to give 13 as a white foam (4.07 g, 11.7 mmol, 77%). ¹H NMR (400 MHz, CDCl₃) δ (ppm) 8.32-8.27 (m, 2H), 7.46-7.40 (m, 2H), 5.56 (t, J=5.4 Hz, 1H), 4.49-4.42 (m, 2H), 4.28 (d, J=8.2 Hz, 2H), 3.80-3.76 (m, 2H), 3.70-3.65 (m, 2H), 3.39-3.30 (m, 2H), 2.36-2.16 (m, 6H), 1.62-1.46 (m, 2H), 1.38 (quintet, J=8.7 Hz, 1H), 1.05-0.92 (m, 2H).

Preparation of 14: To a mixture of 13 (2.61 g, 86.8% pure according to 1H-NMR, 4.62 mmol, 1.0 equiv.) in DCM (80.0 mL) was added 4-nitrophenyl chloroformate (2.14 g, 10.6 mmol, 2.30 equiv.), followed by Et₃N (3.70 mL, 2.68 g, 26.5 mmol, 5.75 equiv.). The reaction mixture was stirred at rt for 4.5 hours and was then concentrated in vacuo, affording a yellow oil. The residue was purified by gradient column chromatography (50→100% EtOAc/heptane+1% AcOH) followed by a 2^{nd} gradient column chromatography purification (50→100% EtOAc/heptane+1% AcOH) to afford 14 as a white solid (2.1 g, 88% pure according to 1H-NMR, 2.25 μmol, 49%). ¹H NMR (400 MHz, DMSO-d6) δ (ppm) 8.33-8.20 (m, 4H), 7.40 (d, J=8.4 Hz, 4H), 5.74 (t, J=5.8 Hz, 1H), 4.56-4.42 (m, 4H), 4.40-4.31 (m, 2H), 4.26 (d, J=8.3 Hz, 2H), 3.83-3.72 (m, 4H), 3.71-3.64 (m, 2H), 3.60 (t, J=4.9 Hz, 2H), 3.34-3.23 (m, 2H), 2.36-2.14 (m. 6H), 1.82-1.44 (m, 4H), 1.44-1.31 (m, 1H), 0.98-0.92 (m, 2H).

### Example 8: Preparation of Compound 17b

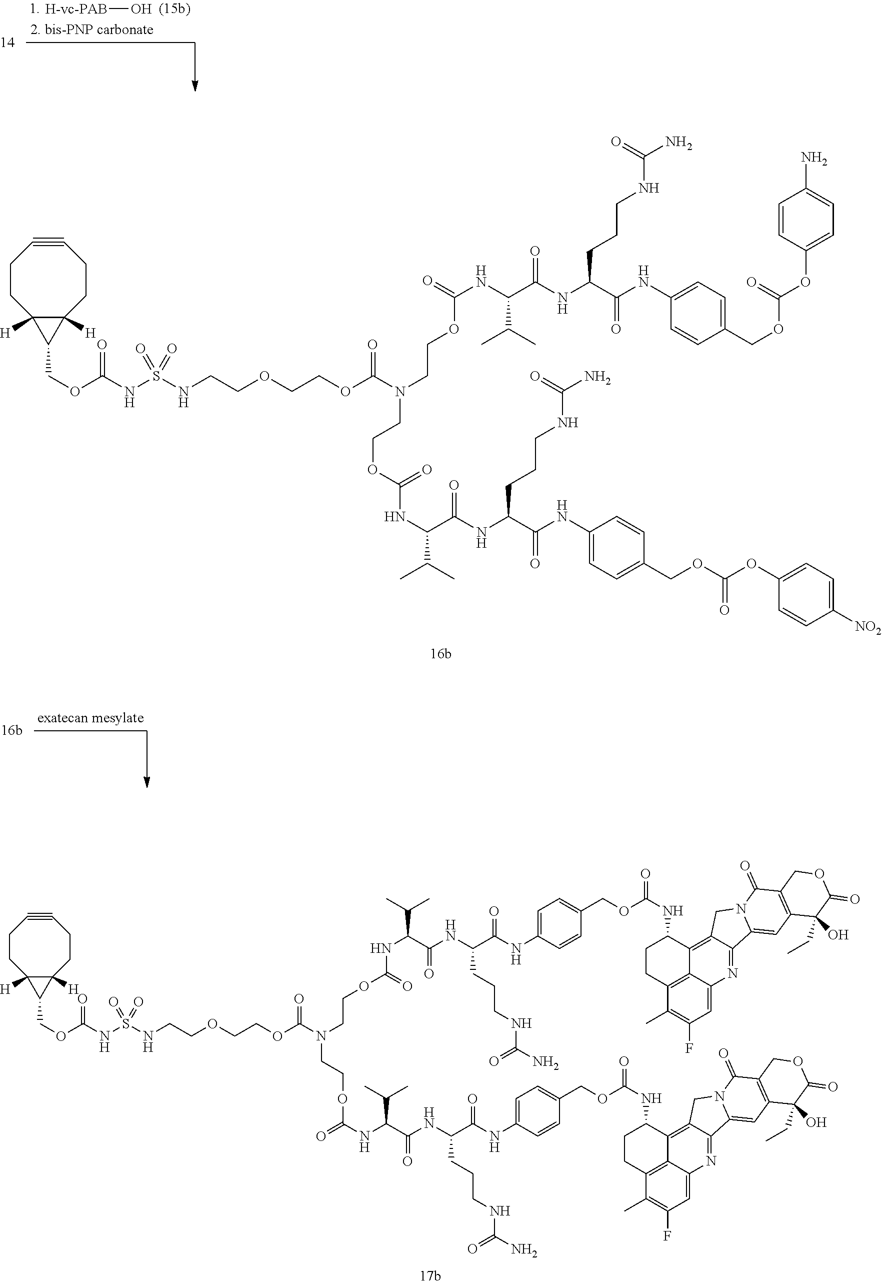

Preparation of 16b: To a stirring solution of 14 (62 mg, 75 μmol) in DMF (500 μL) was added 15b (60 mg, 158 μmol) and Et₃N (63 μL, 46 mg, 450 μmol). After 3.5 h, bis(4-nitrophenyl) carbonate (137 mg, 450 μmol) and Et₃N (63 μL, 46 mg, 450 μmol) were added. The reaction mixture was diluted with DMF (500 μL) and stirring was continued for 1.5 h. The reaction mixture was concentrated and the residue was purified by automated gradient column chromatography over silica gel (0→15% MeOH/DCM) to afford 16b as a white solid (86 mg, 52.7 μmol, 70%). LCMS (ESI+) calculated for C₇₂H₉₄N₁₅O₂₇S⁺ (M+H)⁺ 1632.62, found 1632.70.

Preparation of 17b: To a mixture of exatecan mesylate (2.0 mg, 3.7 μmol) in DMF (40 μL) were added Et₃N (0.9 mg, 1.2 μL, 8.9 μmol) and a solution of compound 16b (2.9 mg, 1.78 μmol) in DMF (25 μL). The reaction mixture was left for 19 h. The reaction mixture was diluted with DMF (100 μL) and purified by preparative HPLC (XBridge prep C18 5 um OBD, 30×100 mm, 30→100% MeCN/H₂O (containing 1% AcOH)) to afford 17b as a white solid (2.5 mg, 1.1 μmol, 62%). LCMS (ESI+) calculated for C₁₀₈H₁₂₉F₂N₁₉O₂₉S₂²⁺ (M/2+H)⁺ 1113.45, found 1113.80.

### Example 9: Preparation of Compound 21b

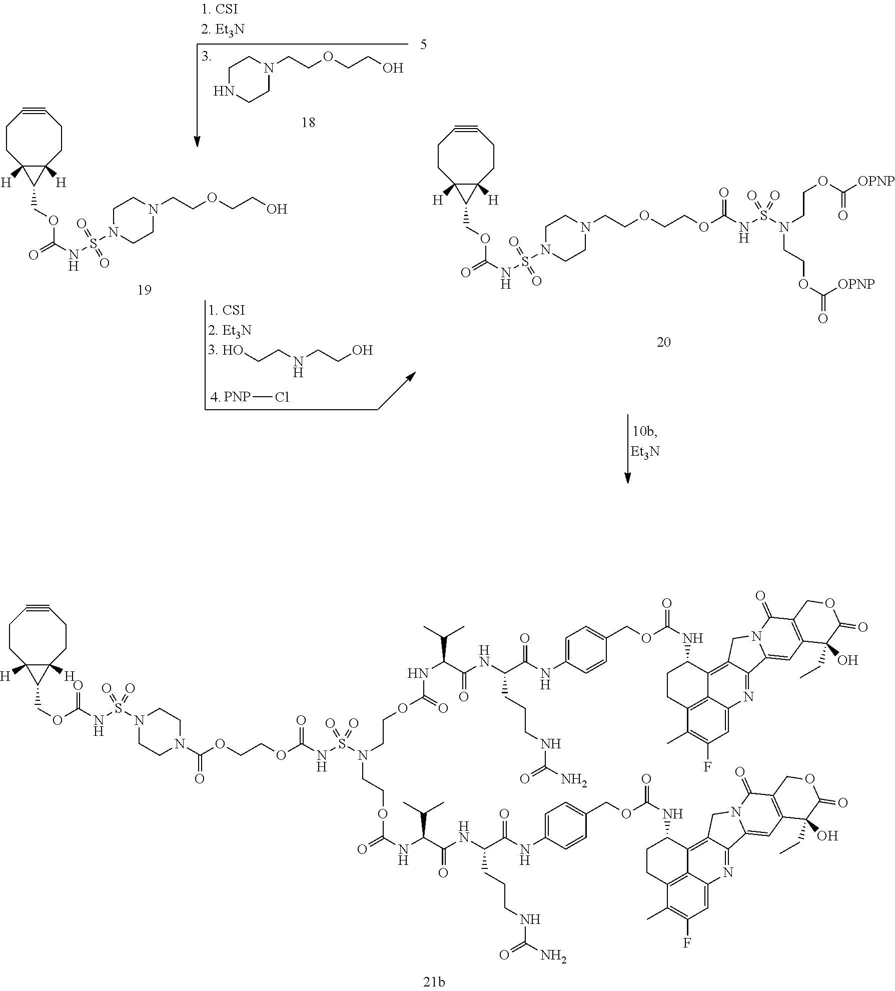

Preparation of 19: To a cooled solution (0° C.) of BCN—OH (5, 1.0 g, 6.7 mmol) in DCM (50 mL), under a N2 atmosphere, was added CSI (597 μL, 0.94 g, 6.7 mmol). After 7 minutes stirring was continued at ambient temperature and additional CSI (58 μL, 0.1 g, 0.67 mmol) was added. After 6 minutes stirring Et₃N (1.86 mL, 1.35 g, 13.3 mmol) was added followed after 3 minutes stirring by addition of 2-[2-(1-piperazinyl)ethoxy]ethanol (18, 1.42 mL, 1.51 g, 8.65 mmol). The mixture was stirred for 17 hours. The reaction mixture was concentrated in vacuo and the residue was purified by automated gradient column chromatography over silica gel (0→10% MeOH/DCM) to afford 19 as light-yellow foam (2.05 g, 3.91 mmol, 58%). 1H NMR (400 MHz, CDCl₃) δ (ppm) 4.21 (d, J=8.2 Hz, 2H), 3.72-3.63 (m, 4H), 3.62-3.56 (m, 2H), 3.47-3.36 (m, 4H), 2.70-2.55 (m, 6H), 2.37-2.15 (m, 6H), 1.66-1.48 (m, 2H), 1.39 (quintet, J=8.7 Hz, 1H), 1.03-0.89 (m, 2H). LCMS (ESI+) calculated for C₁₉H₃₂N₈O₆S⁺ (M+H⁺) 430.20, found 430.43.

Preparation of 20: To a cooled (0° C.) stirring suspension of 19 (750 mg, 1.75 mmol) in DCM (18 mL) was added CSI (167 μL, 272 mg, 1.92 mmol). After stirring for 2 minutes at 0° C. was continued at ambient temperature for 28 minutes. Then Et₃N (1.22 mL, 886 mg, 8.75 mmol) was added and after 3 minutes stirring a solution of diethanolamine (229 mg, 209 μL, 2.18 mmol) in DMF (0.5 mL) was added. After 40 min, bis(4-nitrophenyl) carbonate (1.33 g, 4.38 mmol) and Et₃N (725 μL, 526 mg, 5.20 mmol) were added. After 18 h, additional bis(4-nitrophenyl) carbonate (266 mg) and stirring was continued for 25 h. The reaction mixture was concentrated and the residue was purified by automated gradient column chromatography over silica gel (0→20% MeOH/EtOAc followed by 50% MeOH/DCM). After pooling and concentration of the product containing fractions the residue was re-purified twice by preparative RP-HPLC (XBridge prep C18 5 um OBD, 30×100 mm, 30-90% CH₃CN/H₂O+1% AcOH) to afford 20 as a film (10.5 mg, 10.8 μmol, 0.6%). LCMS (ESI+) calculated for C₃₈H₄₈N7O₁₉S₂²⁺ (M+H⁺) 970.24, found 970.39.

Preparation of 21b: To a solution of compound 20 (10.5 mg, 10.8 μmol) in DMF (270 μL) were added a solution of compound 10b (20.4 mg, 27.1 μmol) in DMF (175 μL) and Et₃N (6.6 mg, 9.1 μL, 65 μmol). The reaction mixture was left for 17.5 h. The reaction mixture was diluted with DCM (2 mL) and purified by automated gradient column chromatography over silica gel (0→20% MeOH/DCM) to afford 21b as a pale-yellow solid (13.5 mg, 6.13 μmol, 57%). LCMS (ESI+) calculated for C₁₀₆H₁₂₅F₂N₇O₁₉S₂²⁺ (M/2+2H)⁺ 1101.41, found 1101.61.

### Example 10: Preparation of Compound 25a

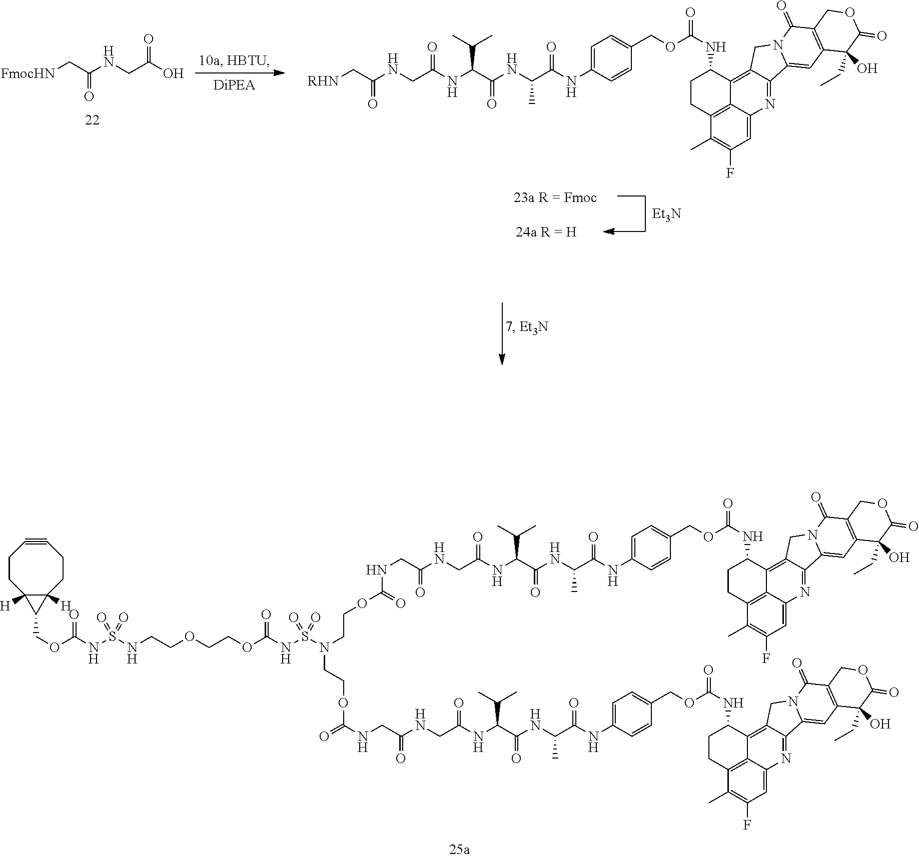

Preparation of 23a: To a solution of 10a (21.7 mg, 28.7 μmol, 1.0 equiv.) and Fmoc-Gly-Gly-OH (12.6 mg, 36.6 μmol, 1.24 equiv.) in dry DMF was added HBTU (18.5 mg, 48.9 μmol, 1.7 equiv.), followed by DiPEA (12.3 mg, 16.5 μL, 94.9 μmol, 3.3 equiv.). The resulting mixture was mixed and left at rt for 110 minutes and then diluted with DCM and the resulting mixture was purified by gradient column chromatography (0→12% MeOH/DCM) to afford 23a as a light-yellow solid (36 mg, 48% purity by HPLC, 16 μmol, 55, 0%). LCMS (ESI+) calculated for C₅₉H₆₀FN₈O₁₂⁺ (M+H⁺) 1091.43, found 1091.61.

Preparation of 24a: Compound 23a (36 mg, 48 wt %, 16 μmol, 1.0 equiv.) was taken up in a mixture of DMF (250 μL) and H₂O (1.25 μL). To the resulting mixture was added Et₃N (8.0 mg, 11 μL, 79 μmol, 5.0 equiv.) and the resulting solution was left at rt for circa 5 hours. Next, additional Et₃N (8.0 mg, 11 μL, 79 μmol, 5.0 equiv.) was added to the reaction mixture and the mixture was left at rt for 18 h and then stored in the freezer for another day. The reaction mixture was removed from the freezer and concentrated in vacuo, followed by co-evaporated with dry DMF (3×) to afford 24a as a brown oil, which was used without further purification. LCMS (ESI+) calculated for C₄₄H₅₀FN₈O₁₀⁺ (M+H⁺) 869.36, found 869.53.

Preparation of 25a: To a vial containing 24a (14 mg, 16 μmol, 3.2 equiv.) was added a 100 mM solution of compound 7 (4.5 mg, 50 μL, 5.0 μmol, 1.0 equiv.) in DMF, followed by Et₃N (2.5 mg, 3.5 μL, 25 μmol, 5.0 equiv.). To the resulting mixture was added additional dry DMF (50 μL) and the reaction mixture was then left at rt for 4.5 hours. Next, the reaction mixture was diluted with dry DMF and then purified by RP-HPLC (C18, 30%→90% MeCN (containing 1% AcOH) in water (containing 1% AcOH) to afford 25a (3.6 mg, 1.5 μmol, 31% yield). LCMS (ESI+) calculated for C₁₁₀H₁₃₀F₂N₂₀O₃₃S²⁺ (M+2H⁺) 1180.93. found 1180.94.

### Example 11: Preparation of Compound 26a

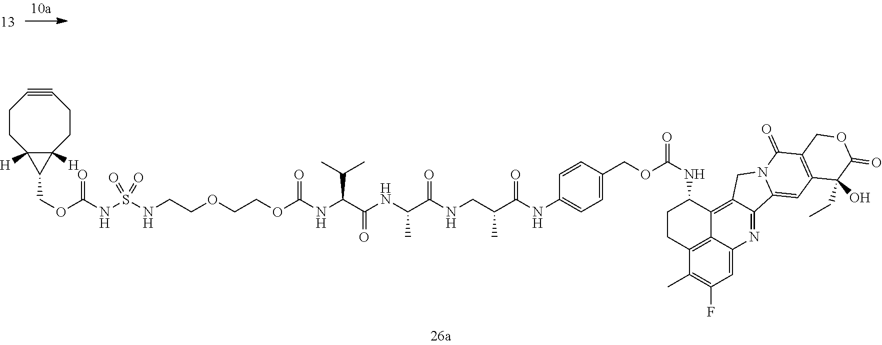 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)CNC(=O)[C@H](C)NC(=O)[C@@H](NC(=O)OCCOCCNS(=O)(=O)NC(=O)OC[C@@H]2[C@@H]4CCC#CCC[C@@H]42)C(C)C)cc1)CC3]

To a solution of 10a (18.9 mg, 0.025 mmol) in anhydrous DMF (500 μL) were added 13 (16 mg, 0.03 mmol) and Et₃N (11 μL, 0.075 mmol). After stirring for 23 h at ambient temperature, the reaction mixture was diluted with DMF till a total volume of 700 μL was reached and purified by preparative RP-HPLC (Column Xbridge prep C18 5 um OBD, 30×100 mm, 30%→100% MeCN/H₂O+1% AcOH). The product 26a was obtained as an off-white solid (5.3 mg, 4.6 μmol, 18.6%). LCMS (ESI+) calculated for C₅₆H₆₆FN₈O₁₅S⁺ (M+H)⁺ 1142.23, found 1142.54.

### Example 12: Preparation of Compounds 31a and 32a

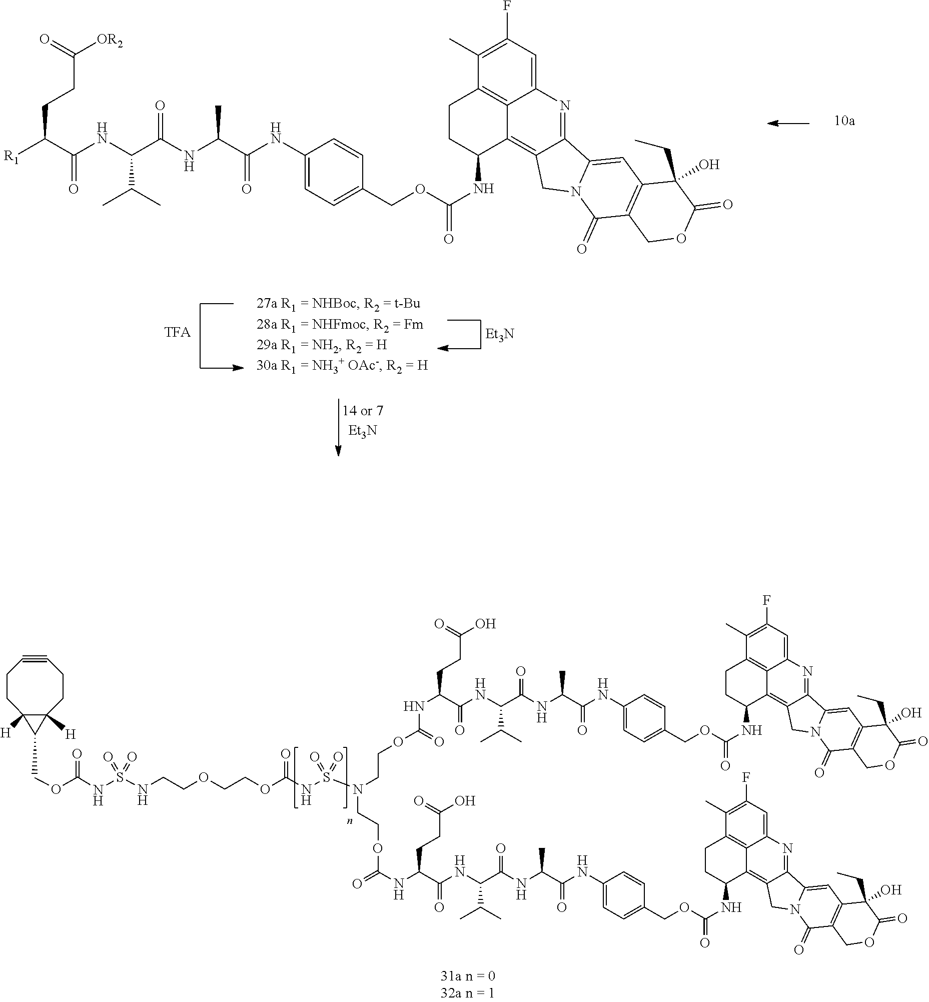

Preparation of 27a: To a vial containing Boc-Glu(OtBu)-OH (11.1 mg, 36.6 μmol, 1.32 equiv.) was added a 148.4 mM solution of 10a (21.0 mg, 187.5 μL, 27.83 μmol, 1.0 equiv.) in dry DMF, followed by HBTU (18.6 mg, 49.0 μmol, 1.76 equiv.) and additional dry DMF (75 μL). Finally, DiPEA (12.2 mg, 16.5 μL, 94.7 μmol, 3.40 equiv.) was added and the reaction mixture was mixed until a brown solution was obtained. The reaction mixture was left at rt for circa 35 minutes and was then diluted with DCM (2.8 mL) and the resulting solution was then purified by gradient column chromatography (0→12% MeOH/DCM) to afford 27a as a white residue (30.8 mg, 29.6 μmol, quantitative yield). LCMS (ESI+) calculated for C₅₄H₆₇FN₇O₁₃⁺ (M+H⁺) 1040.48, found 1040.70.

Preparation of 28a: Fmoc-Glu(OFm)-OH (418.3 mg, 0.93 Eq, 763.8 μmol) was added to a 10 mL container containing a solution of 10a (619.9 mg, 1 Eq, 821.3 μmol) in 2.5 ml dry DMF. To the resulting white suspension was added additional dry DMF (1.0 mL), followed by DIPEA (318.5 mg, 429 μL, 3 Eq, 2.464 mmol). To this suspension was added HBTU (289.7 mg, 0.93 Eq, 763.8 μmol) together with 0.5 mL additional dry DMF. The reaction mixture was mixed for 5 minutes, generating a solution, which was left at rt for another 40 minutes. The reaction mixture was diluted with DCM (40 mL) and the resulting mixture was purified by gradient column chromatography (0 →10% MeOH/DCM) to afford 28a as a white solid (617.5 mg, 481 μmol, 63, 0%). LCMS (ESI+) calculated for C₇₄H₇₁FN₇O₁₃⁺ (M+H⁺) 1284.51, found 1284.91.

Preparation of 29a: To a vial containing 28a (617.5 mg, 481 μmol, 1.0 equiv.) was added DMF (7.5 mL) and Et₃N (670 μL, 486.5 mg, 4.81 mmol, 10 equiv.). The resulting mixture was heated in a water bath to 40° C. for 5 minutes, generating a brown solution, which was left at rt for 18 h. The reaction mixture was then kept in the freezer for 3 days. Next, the reaction mixture was removed from the freezer and concentrated in vacuo, affording 29a as a brown oil which was used without further purification. LCMS (ESI+) calculated for C₄₅H₅₁FN₇O₁₁⁺ (M+H⁺) 884.36, found 884.70.

Preparation of 30a: A vial containing 27a (10.8 mg, 10.4 μmol, 1.0 equiv.) was placed in an ice-bath, followed by the addition of ice-cold TFA (1.04 g, 700 μL, 9.15 mmol, 881 equiv.). The resulting solution was mixed and then left in an ice-bath for 70 minutes. The reaction mixture was then concentrated in vacuo (water-bath at 34° C.) and the residue was taken up in a 1:1 mixture of MeCN and DMSO and then purified by RP-HPLC (C18, 5%→90% MeCN (+1% AcOH) in water (+1% AcOH) to afford 30a as an ammonium acetate salt (4.5 mg, 5.1 μmol, 49% yield). LCMS (ESI+) calculated for C₄₅H₅₁FN₇O₁₁⁺ (M+H⁺) 884.36, found 884.55.

Preparation of 31a: Crude 29a (386 mg, 437 μmol, 3.86 equiv.) was taken up in dry DMF (300 μL), followed by the addition of 14 (105.6 mg, 88 wt % by 1H-qNMR, 113.1 μmol, 1.0 equiv.), additional dry DMF (200 μL) and Et₃N (78.8 μL, 57.2 mg, 565 μmol, 5.0 equiv.). The resulting mixture was mixed, generating a brown solution, which was left at rt for 85 minutes. Next, the reaction mixture was stored in the freezer for 18.5 hours and then removed from the freezer and left at rt for an additional 6 hours before storing the reaction mixture in the freezer for another 18 hours. Finally, the reaction mixture was removed from the freezer and purified by RP-HPLC (C18, 50%→100% MeCN (1% AcOH) in water (1% AcOH). The pure fractions were combined with the pure fractions which were obtained from smaller scale reaction utilizing 29a (38.9 mg, 44.0 μmol, 3.88 equiv.), 14 (10.6 mg, 88 wt % by 1H-qNMR, 11.4 μmol, 1.0 equiv.) and Et₃N (7.91 μL, 5.74 mg, 56.8 μmol, 5.0 equiv.) in 53 μL dry DMF with a reaction time of 5 hours at rt. Concentration of the pure fraction in vacuo affording 31a as an off-white residue (86.6 mg, 37.5 μmol, 33.1%). LCMS (ESI+) calculated for C₁₁₂H₁₃₁F₂N₁₇O₃₃S²⁺ (M+2H⁺) 1156.44, found 1157.03.

Preparation of 32a: To a vial containing of 30a (4.50 mg, 5.09 μmol, 3.28 equiv.) was added a 100 mM solution of compound 7 in dry DMA (1.40 mg, 15.5 μL, 1.55 μmol, 1.0 equiv.), followed by Et₃N (1.26 mg, 1.73 μL, 12.4 μmol 8.00 equiv.). The resulting mixture was vortexed and heated to 43° C. for circa 10 minutes to give an orange solution. The reaction mixture was then left in the dark at rt for 2 hours 45 minutes and then stored in the freezer for 16 hours. Next, the reaction mixture was removed from the freezer and left at rt for 40 minutes before diluting the reaction mixture with DMF. The resulting solution was purified by RP-HPLC (C18, 30%→90% MeCN (+1% AcOH) in water (+1% AcOH) to afford 32a as a white solid (1.0 mg, 0.42 μmol, 27%). LCMS (ESI+) calculated for C₁₁₂H₁₃₂F2N18O₃₅S₂⁺ (M+2H⁺) 1195.93, found 1196.33.

### Example 13: Preparation of Compound 35a

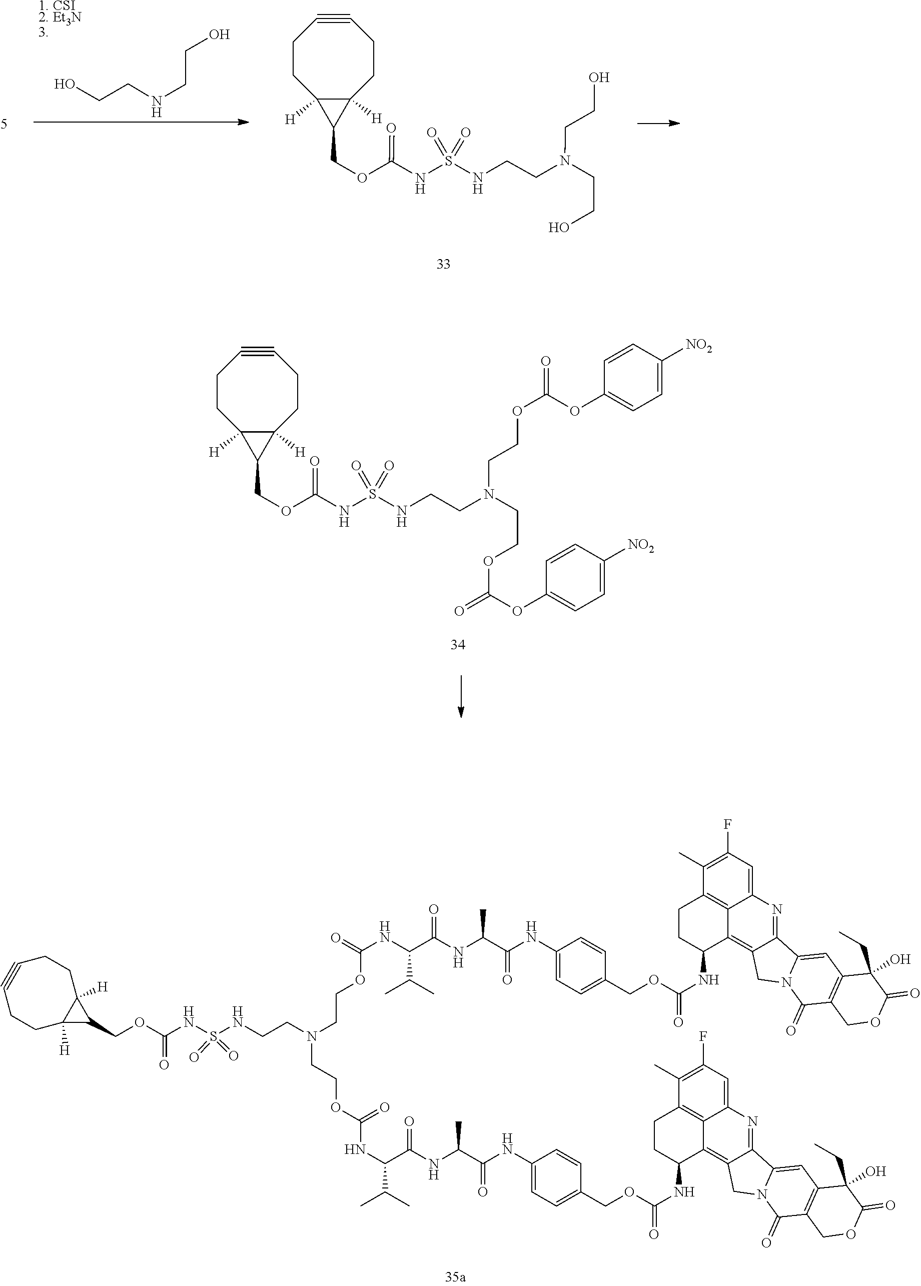

Preparation of 33: Compound 5 (101 mg, 0.67 mmol) was dissolved in DCM (800 μL) and chlorosulfonyl isocyanate (CSI, 64.0 μL, 0.74 mmol) was added, resulting in a brown solution. After stirring for 17 min at ambient temperature, Et₃N (187.0 μL, 1.34 mmol) was added (mixture turned yellow) followed by N,N,-(2-hydroxyethyl)ethylenediamine (110.9 mg, 0.748 mmol) dissolved in DCM (1.0 mL). After stirring for an additional 18 h at ambient temperature, the crude mixture was concentrated in vacuo and purified by automated gradient column chromatography over silica gel (0%→30% MeOH/DCM) to give 33 as a white waxy solid (33.5 mg, 0.083 mmol, 12%). LCMS (ESI+) calculated for C₁₇H₃₀N₃O₆S⁺ (M+H)⁺ 404.50, found 404.42.

Preparation of 34: To a solution of 33 (16.5 mg, 0.041 mmol) in DCM (900 μL) were added bis(4-nitrophenyl) carbonate (29.9 mg, 0.098 mmol) and Et₃N (17.0 μL, 0.012 mmol). After stirring for 96 h at ambient temperature, the crude mixture was concentrated in vacuo and purified by automated gradient column chromatography over silica gel (10%→100% EtOAc/heptane) to give 34 as clear oil (2.5 mg, 0.003 mmol, 8%). LCMS (ESI+) calculated for C₃₁H₃₆N₅O₁₄S⁺ (M+H)⁺ 734.71, found 734.48.

Preparation of 35a: To a solution of 34 (2.5 mg, 0.003 mmol) in DMF (110 μL) were added a 200 mM stock of 10a (41.0 μL, 6.2 mg, 0.008 mmol) and Et₃N (3.0 μL, 0.02 mmol). After 4.5 h at ambient temperature, the reaction mixture was purified by preparative RP-HPLC (Column Xbridge prep C18 5 um OBD, 30×100 mm, 5%→90% MeCN/H₂O+1% AcOH). The product 35a was obtained as a colorless film (1.0 mg, 0.5 μmol, 10%). LCMS (ESI+) calculated for C₉₉H₁₁₂F₂N₁₅O₂₄S⁺ (M+H)⁺ 1966.10, found 1966.85.

### Example 14: Preparation of Compound 39a

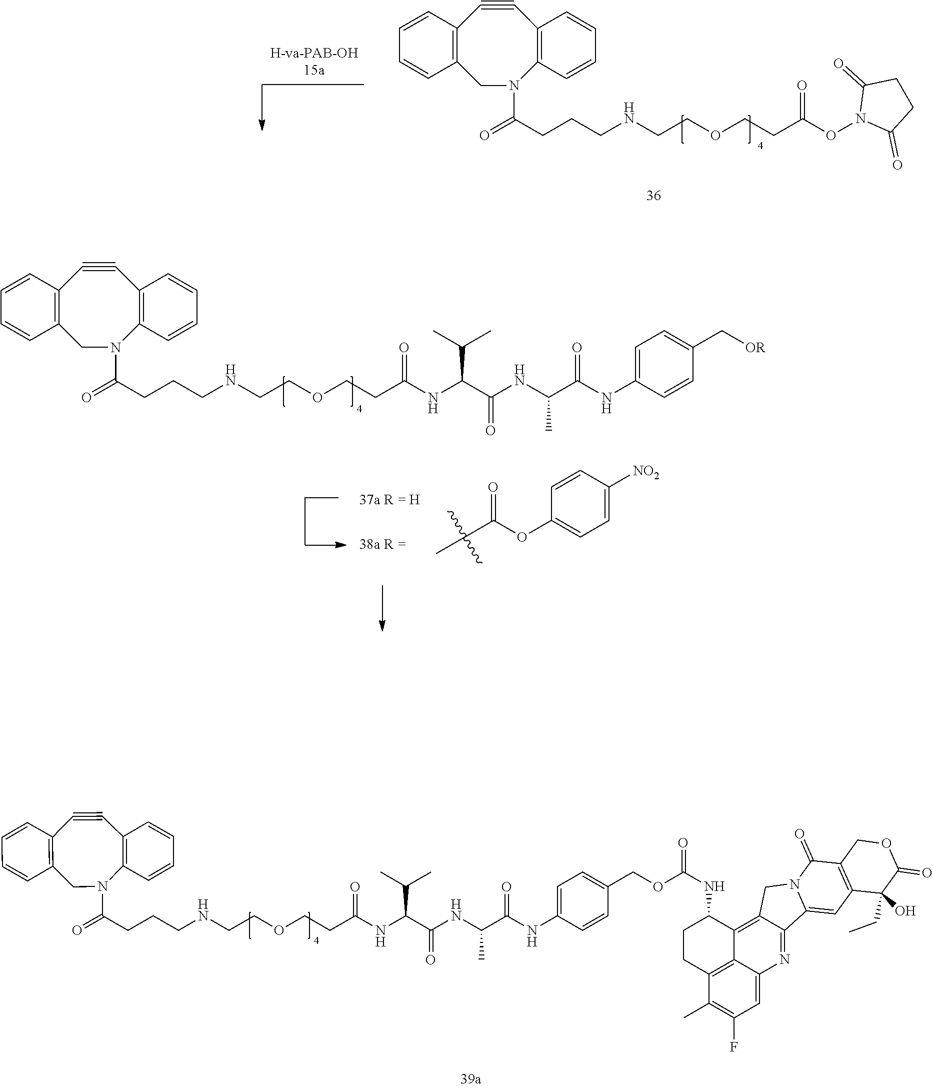

Preparation of 37a: To a solution of DBCO-PEG₄-OSu (36, 63.0 mg, 0.097 mmol) in DMF (2.0 mL) were added Val-Ala-PAB (15a, 26.0 mg, 0.108 mmol) followed by Et₃N (41.0 μL, 0.28 mmol). After stirring for 2 h at ambient temperature, more Val-Ala-PAB (15a, 7.8 mg, 0.03 mmol) dissolved in DMF (150 μL) was added. After an additional 45 min, the reaction mixture was concentrated to 0.5 mL volume and purified by automated gradient column chromatography over silica gel (0%→15% MeOH/DCM) to give 37a as clear oil (94 mg, 0.113 mmol). ¹H NMR (400 MHz, CDCl₃) δ (ppm) 7.72-7.61 (m, 3H), 7.44-7.21 (m, 9H), 5.12 (d, J=14.0 Hz, 1H), 4.74-4.56 (m, 3H), 4.24-4.14 (m, 1H), 3.83-3.73 (m, 1H), 3.70 (dd, J=14.0 Hz; J=1.8 Hz, 1H), 3.66-3.36 (m, 16H), 3.35-3.14 (m, 2H), 2.70-1.80 (m, 6H), 1.49-1.36 (m, 3H), 1.05-0.90 (m, 6H).

Preparation of 38a: To a solution of 37a (94.0 mg, 0.113 mmol) in DMF (1.5 mL) were added bis(4-nitrophenyl) carbonate (38.0 mg, 0.12 mmol) and Et₃N (46.0 μL, 0.33 mmol). After stirring for 2 h, extra bis(4-nitrophenyl) carbonate (10.0 mg, 0.03 mmol) was added. After an additional hour, the reaction mixture was concentrated until 1 mL solvent was left and purified by automated gradient column chromatography over silica gel (100% DCM followed by 0%→10% MeOH/DCM) to give 38a as clear oil (79 mg, 0.095 mmol). LCMS (ESI+) calculated for C₅₂H₆₀N6O₁₄⁺ (M+H)⁺ 994.07, found 994.74.

Preparation of 39a: To a solution of exatecan free base (7.28 mg, 0.0167 mmol, as recovered in the preparation of 9a, see example 3) in DMF (214 μL) were added 38a (15.1 mg, 0.015 mmol) and Et₃N (6.0 μL, 0.04 mmol). After 16.5 h at ambient temperature, the reaction mixture was diluted with DMF till 600 μL and purified by preparative RP-HPLC (Column Xbridge prep C18 5 um OBD, 30×100 mm, 30%→100% MeCN/H₂O+1% AcOH). The product 39a was obtained as a colorless film (8.3 mg, 15.2 μmol, 42%). LCMS (ESI+) calculated for C₇₀H₇₈FN₈O₁₅⁺ (M+H)⁺ 1290.41, found 1290.02.

### Example 15: Preparation of Compound 43a

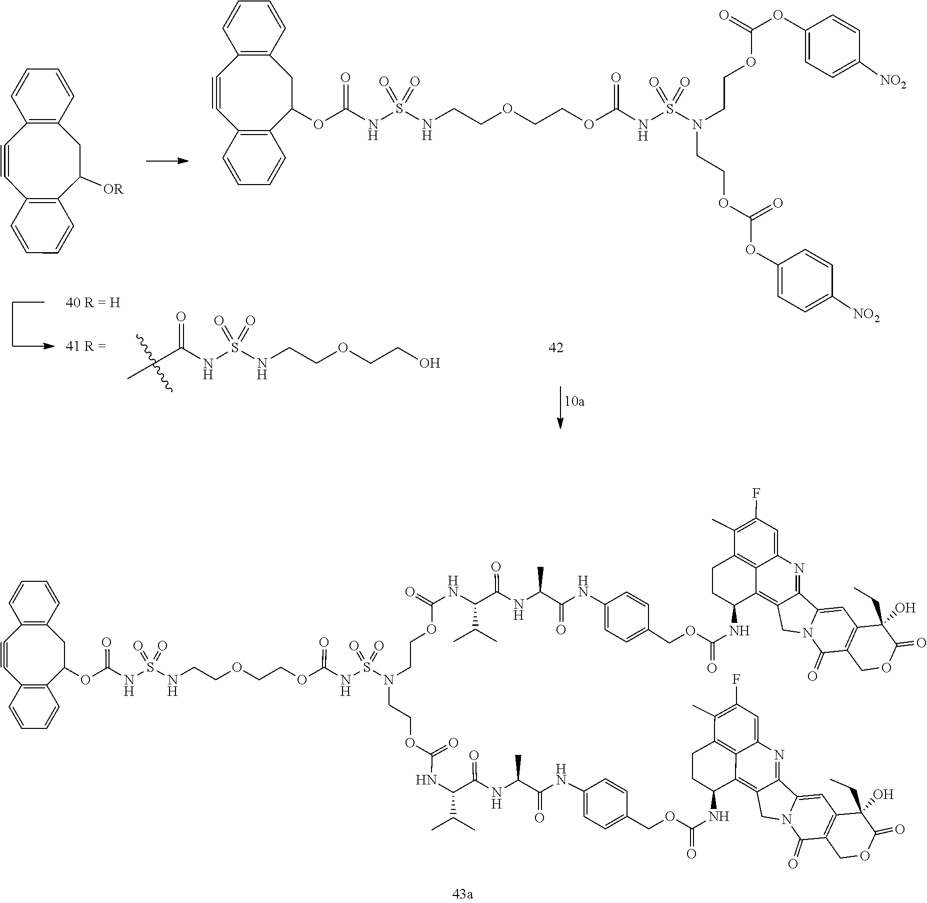

Preparation of 41: To a solution of DIBO (40, 220 mg, 1.0 mmol) in anhydrous DCM (15 mL) was added chlorosulfonyl isocyanate (CSI, 88.1 μL, 1.0 mmol), which formed a white suspension. After stirring for 20 min at ambient temperature, Et₃N (282 μL, 2.0 mmol) was added followed by 2-amino-ethoxyethanol (117 mg, 1.11 mmol). After stirring for an additional 20 min, the reaction mixture was quenched through addition of aqueous NH₄Cl (sat., 30 mL). After separation, the aqueous layer was extracted with DCM (20 mL). The combined organic layers were dried (MgSO₄) and concentrated. The residue was purified by automated gradient column chromatography over silica gel (0%→7% MeOH/DCM) to give 41 as a light-yellow oil (498 mg, 1.15 mmol). ¹H NMR (400 MHz, CDCl₃) δ (ppm) 7.52-7.42 (m, 1H), 7.35-7.29 (m, 2H), 7.28-7.19 (m, 5H), 5.42-5.39 (bs, 1H), 3.48-3.43 (m, 4H), 3.36-3.32 (m, 2H), 3.23-3.18 (m, 2H), 3.17-3.12 (m, 2H).

Preparation of 42: To a solution of 41 (192 mg, 0.44 mmol) in DCM (30 mL), under a N₂ atmosphere, was added dropwise chlorosulfonyl isocyanate (CSI, 38.8 μL, 0.44 mmol). After stirring for 20 min at ambient temperature, Et₃N (311 μL, 2.23 mmol) was added and this mixture was stirred for 10 min. After which a solution of diethanolamine (51.6 μL, 0.53 mmol) in DMF (1.5 mL) was added. The reaction was stirred for 1 h followed by 4-nitrophenyl chloroformate (180 mg, 0.89 mmol) and Et₃N (187 μL, 1.34 mmol). After stirring for 18 h at ambient temperature, the reaction mixture was purified by automated gradient column chromatography over silica gel (0%→6% MeOH/DCM) to give 42 as a white solid (187 mg, 0.19 mmol). LCMS (ESI+) calculated for C₄₀H₃₉N₆O₁₉S₂⁺ (M+H)⁺ 971.90, found 971.26.

Preparation of 43a: To a solution of 10a (15 mg, 0.02 mmol) in anhydrous DMF (110 μL) were added 42 (7.9 mg, 0.008 mmol) and Et₃N (5.0 μL, 0.04 mmol). After 6 h at ambient temperature, the reaction mixture was purified by automated gradient column chromatography over silica gel (0%→15% MeOH/DCM) to give 43a as an off-white solid (10.1 mg, 0.0046 mmol, 55%). LCMS (ESI+) calculated for C₁₀₈H₁₁₅F2N16O₂₉S₂⁺ (M/2+H)⁺ 1101.64, found 1101.97.

### Example 16: Preparation of Compound 49a

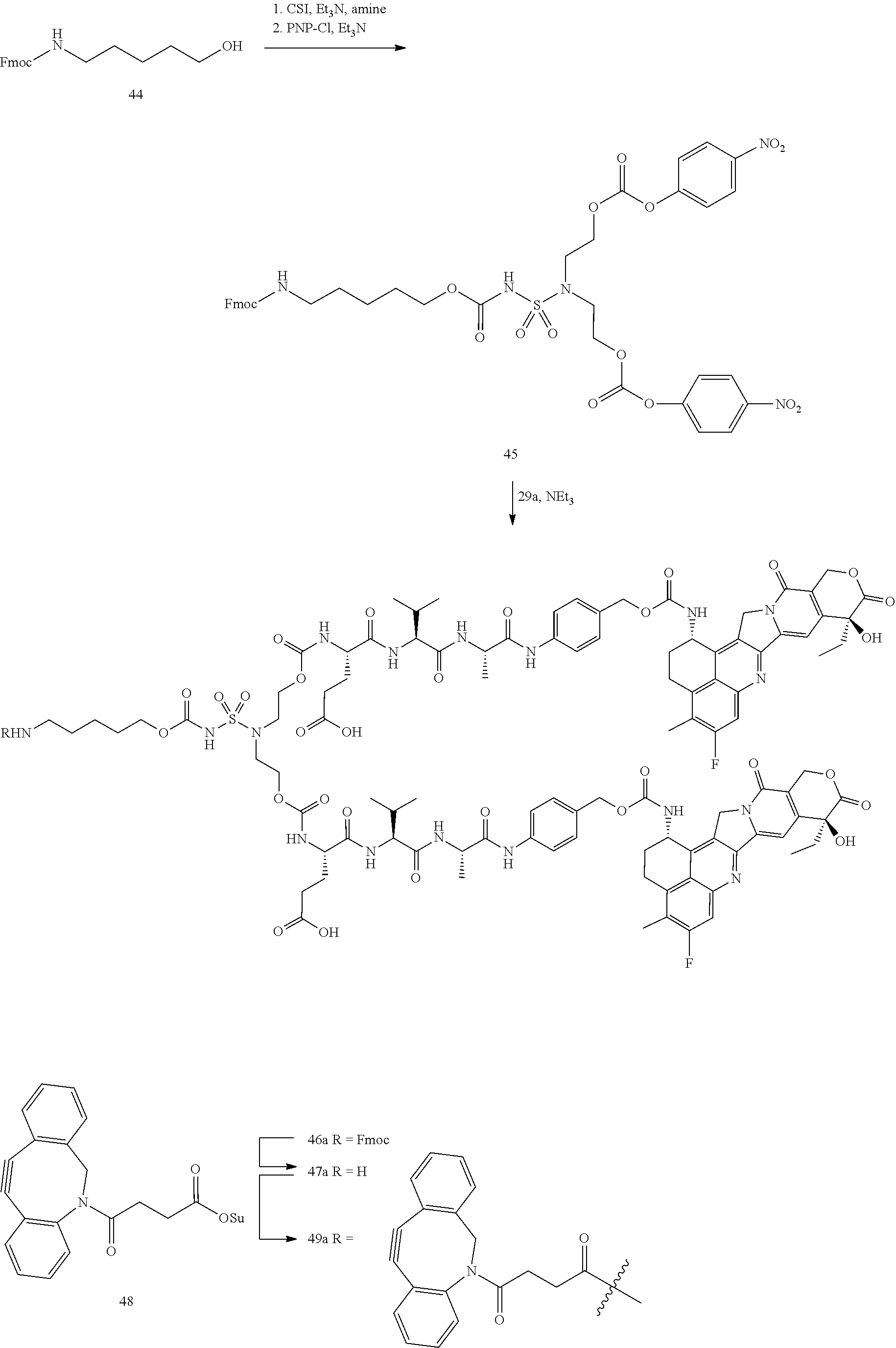

Preparation of 45: To a solution of (9H-fluoren-9-yl)methyl (5-hydroxypentyl)carbamate (397 mg, 1.22 mmol, 1.0 equiv.) in DCM (55 mL) was added dropwise CSI (106 μL, 1.22 mmol, 1.0 equiv.) at rt. The reaction mixture was stirred at rt for 20 minutes, followed by the addition of Et₃N (850 μL, 6.10 mmol, 5.0 equiv.). The resulting mixture was stirred at rt for 10 minutes and then a solution of diethanolamine (144 mg, 1.37 mmol, 1.11 equiv.) in DMF (1.7 mL) was added. The reaction mixture was stirred for 2 hours and then 4-nitrophenyl chloroformate (492 mg, 2.44 mmol, 2.0 equiv.) was added together with additional Et₃N (340 μL, 2.44 mmol, 2.0 equiv.). The reaction mixture was stirred for 18 h and was then concentrated in vacuo and the resulting residue was taken up in DCM (100 mL) and washed with sat. aq. NH₄Cl (50 mL). The water layer was extracted with DCM (50 mL) and the combined organic layers were dried over MgSO₄ and concentrated in vacuo. The residue was then purified by gradient column chromatography (0→5% MeOH/DCM), followed by a second column chromatography purification (65% EtOAc in Heptane) to afford 45 as a white powder (342 mg, 395 μmol, 32, 3% yield). ¹H NMR (400 MHz, CDCl₃) δ (ppm) 8.24 (d, J=9.2 Hz, 4H), 7.76 (d, J=7.5 Hz, 2H), 7.59 (d, J=7.5 Hz, 2H), 7.42-7.33 (m, 6H), 7.33-7.27 (m, 2H), 4.86 (t, J=5.5 Hz, 1H), 4.51 (t, J=5.4 Hz, 4H), 4.43 (d, J=6.9 Hz, 2H), 4.21 (t, J=6.8 Hz, 1H), 4.17-4.09 (m, 2H), 3.84 (t, J=4.4 Hz, 4H), 3.24-3.14 (m, 2H), 1.72-1.62 (m, 2H), 1.62-1.48 (m, 3H), 1.48-1.36 (m, 2H).

Preparation of 46a: A 25 mM solution of crude 29a in dry DMF (25 mg, 1.1 mL, 28 μmol, 4.0 equiv.) containing Et₃N (37.7 μL, 27.3 mg, 270 μmol, 10 equiv.) was concentrated in vacuo. To the resulting residue was added a solution of 45 (6.1 mg, 7.1 μmol, 1.0 equiv.) in dry DMF (150 μL), followed by Et₃N (6.0 μL, 4.2 mg, 42 μmol, 6.0 equiv.). The resulting mixture was heated to 45° C. for 5 minutes and was then left at rf for 2.5 hours and then stored in the freezer for 17 hours. Next, the reaction mixture was removed from the freezer, diluted with dry DMF and filtered over a membrane filter and then purified by RP-HPLC (C18, 50%→100% MeCN (+1% AcOH) in water (+1% AcOH) to afford 46a (7.4 mg, 3.1 μmol, 44% yield). LCMS (ESI+) calculated for C₁₁₇H₁₃₁F₂N₁₇O₃₂S²⁺ (M+2H⁺) 1178.44, found 1178.79.

Preparation of 49a: To a vial containing 46a (7.4 mg, 3.1 μmol, 1.0 equiv.) was added DMF (200 μL), followed by Et₃N (4.4 μL, 3.2 mg, 31.6 μmol, 10 equiv.). The resulting mixture was left at rt for 2 hours, followed by the addition of additional Et₃N (10 μL, 7.3 mg, 71.7 μmol, 23 equiv.). The reaction mixture was mixed and left for 21 hours at rt. Next, DBCO-NHS ester 48 (2.1 mg, 5.2 μmol, 1.7 equiv.) in DMF (5 μL) was added. The resulting brown solution was left at rt for 1 hour and was then stored in the freezer for 18 h. The next day, the reaction mixture was removed from the freezer and then purified by RP-HPLC (C18, 50%→100% MeCN (+1% AcOH) in water (+1% AcOH) to afford impure product, which was further purified by gradient column chromatography (0→30% MeOH/DCM) to afford 49a as a white solid (2.5 mg, 1.0 μmol, 33% yield). LCMS (ESI+) calculated for C₁₂₁H₁₃₄F₂N₁₈O₃₂S²⁺ (M+2H⁺) 1210.96, found 1211.20.

### Example 17: Preparation of Compound 52a

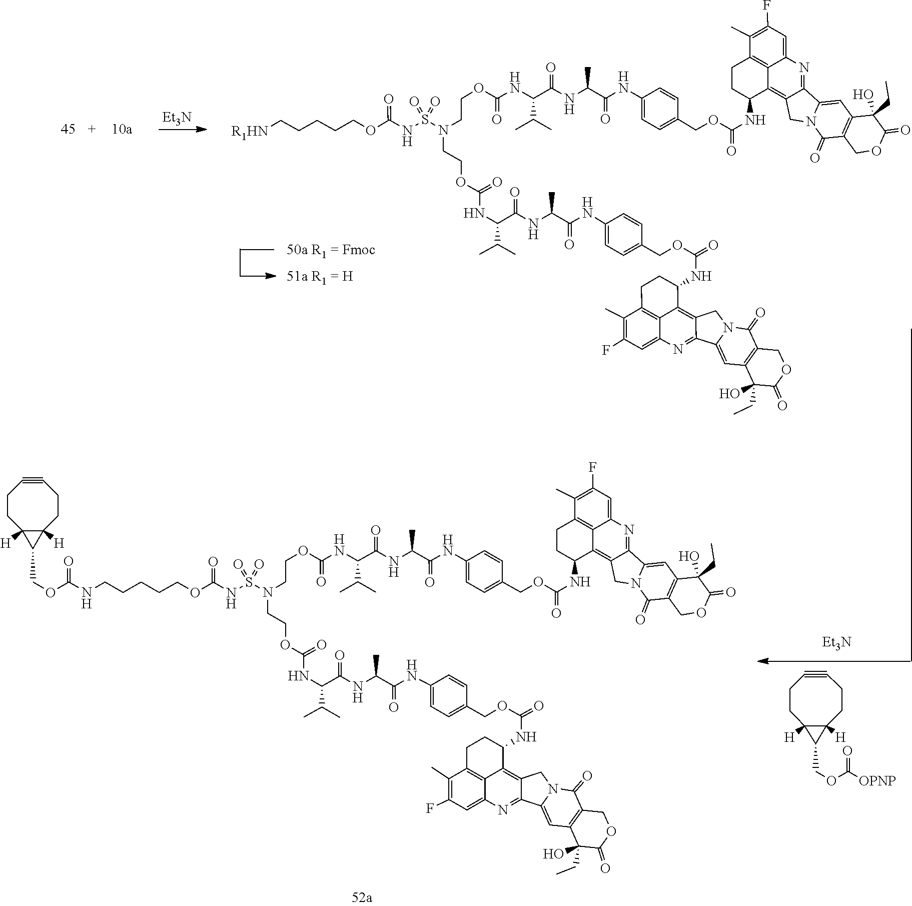

Preparation of 50a: To a vial containing compound 10a (87.3 μmol, 1.71 equiv.) was added 45 (16.2 mg, 18.7 μmol, 1.0 equiv.), followed by dry DMF (200 μL) and Et₃N (9.5 mg, 13 μL, 94 μmol, 5.0 equiv.). The resulting brown solution was left at rt for 50 minutes and was then transferred to a vial containing additional intermediate 10a (55.3 μmol, 3.0 equiv.). The resulting reaction mixture was left at rt for 40 minutes, followed by the addition of additional dry DMF (150 μL). The reaction mixture was left at rt for 20 hours and was then stored in the freezer for 3 days. Next, the reaction mixture was removed from the freezer and 2,2′-(ethane-1,2-diylbis(oxy))bis(ethan-1-amine) (5.0 μL) was added. The reaction mixture was left at rt for 20 minutes and was then diluted with dry DMF and filtered over a 0.2 μm nylon syringe filter and then purified by RP-HPLC (018, 50%→100% MeCN (+1% AcOH) in water (+1% AcOH) to afford impure product, which was further purified by gradient column chromatography (0→30% MeOH/DCM) to afford 50a as a white residue (6.5 mg, 3.06 μmol, 16% yield). LCMS (ESI+) calculated for C₁₀₇H₁₁₇F₂N₁₅O₂₆S²⁺ (M+2H⁺) 1049.40, found 1049.28.

Preparation of 52a: To a vial containing 50a (1.6 mg, 0.76 μmol, 1.0 equiv.) was added DMF (150 μL), followed by Et₃N (1.5 mg, 2.1 μL, 15 μmol, 20 equiv.). The resulting light-brown solution was left at rt for 50 minutes, followed by the addition of additional Et₃N (3.5 μL). The reaction mixture was left at rt for another 18 hours and was then concentrated in vacuo. To this crude residue was added a solution of ((1R,8S,9s)-bicyclo[6.1.0]non-4-yn-9-yl)methyl (4-nitrophenyl) carbonate (BCN—OPNP, 1.2 mg, 3.8 μmol, 5.0 equiv.) in dry DMF (10 μL), followed by the addition of Et₃N (0.35 mg, 0.48 μL, 3.4 μmol, 4.5 equiv.). The resulting brown solution was vortexed and left at rt for 140 minutes. The reaction mixture was then diluted with DCM and the resulting mixture was purified by gradient column chromatography (0→10% MeOH/DCM) to afford 52a as a white solid (1.7 mg, 86% pure according to HPLC, 0.71 μmol, 94%). LCMS (ESI+) calculated for C₁₀₃H₁₁₉F₂N₁₅O₂₆S²⁺. (M+2H⁺) 1026.41, found 1026.11.

### Example 18: Preparation of Compound 54a

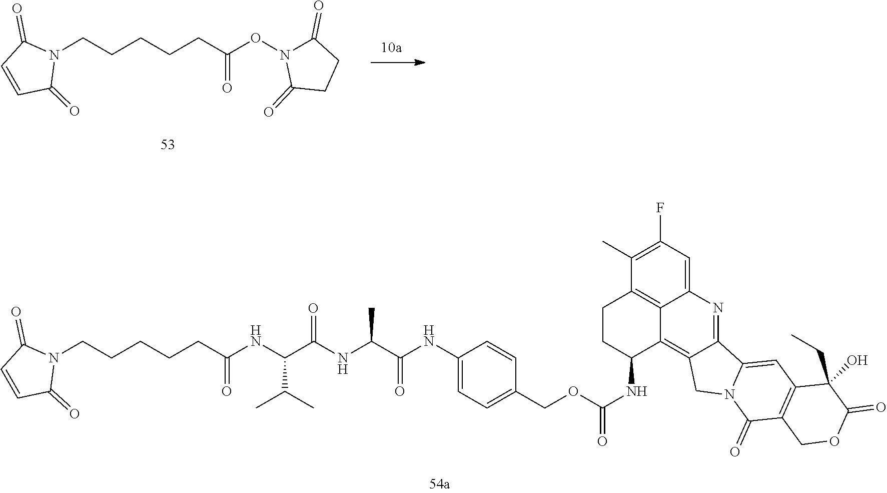 [SMILES: O=C(CCCCCN1C(=O)C=CC1=O)ON1C(=O)CCC1=O ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CCCCCN2C(=O)C=CC2=O)C(C)C)cc1)CC3]

To a solution of 10a (15 mg, 0.020 mmol) in anhydrous DMF (150 μL) were added 2,5-dioxypyrrolidin-1-yl 6-(2,5-dioxo-2,5-dihydro-1H-pyrrol-1-yl) hexanoate (53, 5.5 mg, 0.018 mmol) and DIPEA (10 μL, 0.060 mmol). After 35 min at ambient temperature, the reaction mixture was purified by preparative RP-HPLC (Column Xbridge prep C18 5 um OBD, 30×100 mm, 30%→100% MeCN/H₂O+1% AcOH). The product 54a was obtained as a clear oil (2.4 mg, 2.1 μmol, 11%). LCMS (ESI+) calculated for C₅₀H₅₅FN₇O₁₁⁺ (M+H)⁺ 949.01, found 949.82.

### Example 19: Preparation of Compound 57b

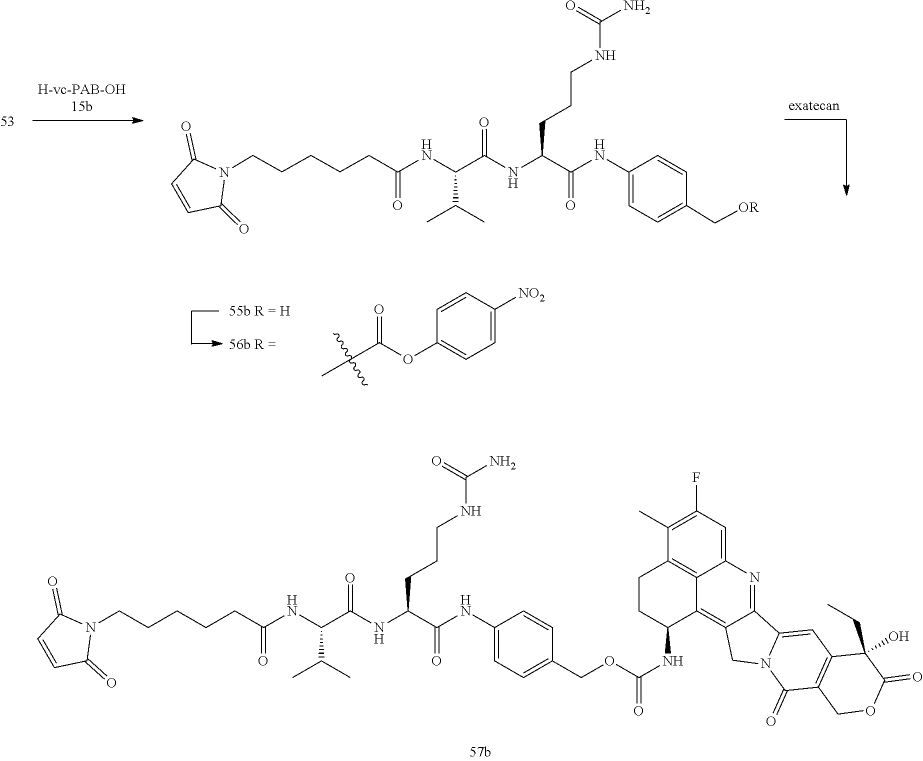

Preparation of 55b: To a vial containing compound 53 (684 μmol) in dry DMF (2 mL) was added H-Val-Cit-PAB-OH (15b, 259 mg, 684 μmol, 1.0 equiv.) and the resulting solution was stirred for 17 hours at rt. Next, the reaction mixture was diluted with additional DMF (2.0 mL) and then poured in 80 mL Et₂O and filtered. The filtered solid was washed with Et₂O (25 mL, 2×) and then concentrated in vacuo to afford 55b as a white solid (378 mg, 95, 3% pure by 1H-NMR, 629 μmol, 91.9% yield). ¹H NMR (400 MHz, DMSO-d6) δ (ppm) 9.91 (s, 1H), 8.08 (d, J=7.6 Hz, 1H), 7.82 (d, J=8.4 Hz, 1H), 7.56 (d, J=8.3 Hz, 2H), 7.24 (d, J=8.2 Hz, 2H), 7.02 (s, 2H), 6.06-5.90 (m, 1H), 5.42 (s, 2H), 5.11 (t, J=5.6 Hz, 1H), 4.52-4.33 (m, 3H), 4.20 (t, J=7.6 Hz, 1H), 3.09-2.93 (m, 2H), 2.26-2.06 (m, 2H), 2.05-1.91 (m, 1H), 1.77-1.65 (m, 1H), 1.65-1.56 (m, 1H), 1.56-1.30 (m, 6H), 1.28-1.15 (m, 2H), 0.93-0.76 (m, 6H).

Preparation of 56b: To a solution of 55b (214 mg, 95, 3% pure by 1H-NMR, 0.357 mmol) and bis(4-nitrophenyl) carbonate (217 mg, 0.714 mmol) in DMF (2.0 mL) was added DIPEA (88 μL, 0.535 mmol). After stirring for 2 h at ambient temperature, the reaction mixture was poured in diethyl ether (80 mL) and filtered. The residue was suspended in diethyl ether (2×50 mL) and filtered again. After concentrating in vacuo, 56b was obtained as a light-yellow solid (251.0 mg, 0.34 mmol, 95%). LCMS (ESI+) calculated for C₃₅H₄₄N₇O₁₁⁺ (M+H)⁺ 738.76, found 738.25. ¹H NMR (400 MHz, DMSO-d6) δ (ppm) 10.08 (s, 1H), 8.40-8.28 (m, 2H), 8.12 (d, J=7.6 Hz, 1H), 7.82 (d, J=8.7 Hz, 1H), 7.67 (d, J=8.5, 2H), 7.62-7.53 (m, 2H), 7.42 (d, J=8.4 Hz, 2H), 7.02 (s, 2H), 5.99 (t, J=5.4 Hz, 1H), 5.43 (s, 2H), 5.26 (s, 2H), 4.46-4.34 (m, 1H), 4.21 (t, J=7.4 Hz, 1H), 3.12-2.88 (m, 2H), 2.26-2.04 (m, 2H), 2.04-1.89 (m, 1H), 1.77-1.67 (m, 1H), 1.67-1.56 (m, 1H), 1.56 (m, 6H), 1.27-1.14 (m, 2H), 0.94-0.78 (m, 6H).

Preparation of 57b: To a solution of exatecan mesylate salt (20 mg, 0.038 mmol) in anhydrous DMF (220 μL) were added DIPEA (33.0 μL, 0.19 mmol) and 56b (25 mg, 0.034 mmol). After 2 h at ambient temperature, the crude reaction mixture was purified directly by preparative RP-HPLC (Column Xbridge prep C18 5 um OBD, 30×100 mm, 30%→90% MeCN/H₂O+1% AcOH). The product 57b was obtained as a clear oil (8.1 mg, 7.8 μmol, 21%). LCMS (ESI+) calculated for C₅₃H₆₁FN₉O₁₂⁺ (M+H)⁺ 1035.10, found 1035.82.

### Example 20: Preparation of Compound 59b

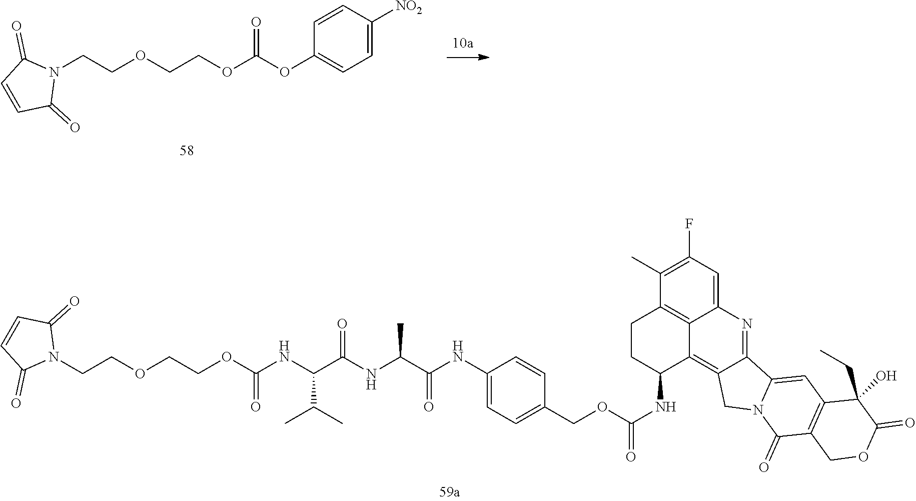 [SMILES: O=C(OCCOCCN1C(=O)C=CC1=O)Oc1ccc([N+](=O)[O-])cc1 ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)OCCOCCN2C(=O)C=CC2=O)C(C)C)cc1)CC3]

To a solution of 10a (15 mg, 0.020 mmol) in anhydrous DMF (150 μL) were added maleimide-PEG1-OPNP (58, 7.7 mg, 0.022 mmol) and DIPEA (10 μL, 0.060 mmol). After 2.5 h at ambient temperature, the crude reaction mixture was purified directly by preparative RP-HPLC (Column Xbridge prep C18 5 um OBD, 30×100 mm, 30%→100% MeCN/H₂O+1% AcOH). The product 59a was obtained as a clear oil (3.6 mg, 3.4 μmol, 17%). LCMS (ESI+) calculated for C₄₉H₅₃FN₇O₁₃+(M+H)⁺ 966.98, found 966.77.

### Example 21: Preparation of Compound 60a

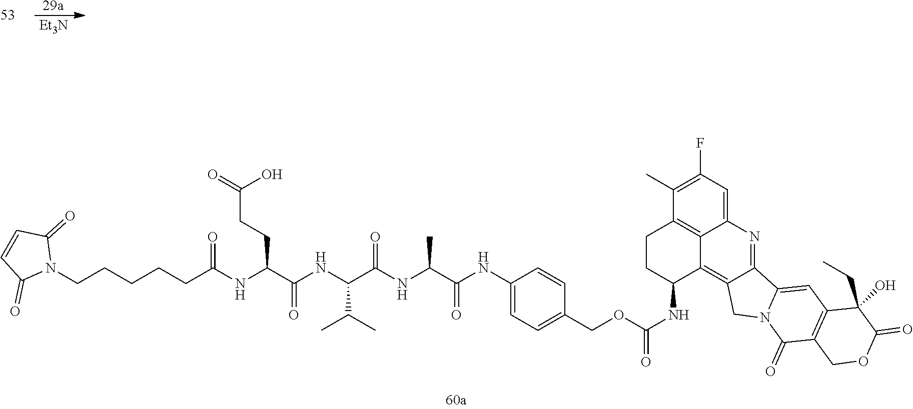 [SMILES: "Et3N" ; "29a" ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCC(=O)O)NC(=O)CCCCCN2C(=O)C=CC2=O)C(C)C)cc1)CC3]

To a vial containing maleimidocaproic acid NHS ester (53, 5.2 mg, 17 μmol, 1.0 equiv.) was added a 25 mM solution of crude 29a in dry DMF (15 mg, 0.68 mL, 17 μmol, 1.0 equiv.) containing Et₃N (23.3 μL, 16.9 mg, 167 μmol, 10 equiv.). The resulting brown solution was left at rt for 40 minutes and then additional maleimidocaproic acid NHS ester (2.1 mg, 6.8 μmol, 0.4 equiv.) in DMF (20 μL) was added. The RM was left at rt for another 24 minutes and then purified by RP-HPLC (C18, 40%→100% MeCN (1% AcOH) in water (1% AcOH) to afford 60a as a white residue (3.3 mg, 2.5 μmol, 80% purity by HPLC, 14% yield). LCMS (ESI+) calculated for C₅₅H₆₂FN₈O₁₄⁺ (M+H⁺) 1077.44, found 1077.78.

### Example 22. Preparation of ADC1a-DAR4 (for Structure See FIG. 9)

A bioconjugate according to the invention was prepared by conjugation of compound 1a as linker-conjugate to azide-modified trastuzumab as biomolecule. Thus, to a solution of trastuzumab-(6-N3-GalNAc)₂ (604 μL, 20.6 mg, 34.1 mg/ml in PBS pH 7.4, trast-v1), prepared according to WO2016170186, was added PBS pH 7.4 (63 μL), 1,2-propylene glycol (587 μL) and compound 1a (80 μL, 10 mM solution in DMF). The reaction was incubated at rt overnight followed by dialysis to PBS pH 7.4. Residual free payload was removed by addition of charcoal (1.2 mg of charcoal per 1 mg of ADC) followed by rotating overnight at rt. Charcoal was removed by centrifugation and filtration and the ADC was purified on an AKTA Purifier-10 (GE Healthcare) with a Superdex200 Increase 10/300 GL (GE Healthcare) column. Mass spectral analysis of the IdeS-digested sample showed one major product (observed mass 26494 Da, approximately 60% of total Fc/2 fragment, calculated mass 26495 Da), corresponding to the conjugated Fc/2 fragment (2× closed lactone form of the payload), and one minor product (observed mass 26510 Da, approximately 30% of total Fc/2 fragment, calculated mass 26513 Da), corresponding to the conjugated Fc/2 fragment (1× closed lactone form and 1× open carboxylate form of the payload).

ADC1a-DAR8 (for structure see FIG. 9) was prepared in the same way by conjugation of compound 1a to trastuzumab-(HC-L¹⁹⁶N mutant)-(6-N₃-GalNAc)₄ (trast-v5).

### Example 23. Preparation of ADC2 (DAR4)

A bioconjugate according to the invention was prepared by conjugation of compound 2 as linker-conjugate to azide-modified trastuzumab as biomolecule. Thus, to a solution of trastuzumab-(6-N3-GalNAc)₂ (363 μL, 12.0 mg, 33.1 mg/ml in PBS pH 7.4, trast-v1), prepared according to WO2016170186, was added PBS pH 7.4 (37 μL), 1,2-propylene glycol (336 μL) and compound 2 (64 μL, 10 mM solution in DMF). The reaction was incubated at rt overnight followed by addition of additional 2 (16 μL, 10 mM solution in DMF) and incubation at rt overnight. The reaction was dialyzed to PBS pH 7.4 and residual free payload was removed by addition of charcoal (4.8 mg of charcoal per 1 mg of ADC) followed by rotating for 4 hours at rt. Charcoal was removed by centrifugation and filtration and the ADC was purified on an AKTA Purifier-10 (GE Healthcare) with a Superdex200 Increase 10/300 GL (GE Healthcare) column. Mass spectral analysis of the IdeS-digested sample showed one major product (observed mass 26847 Da, approximately 60% of total Fc/2 fragment, calculated mass 26847 Da), corresponding to the conjugated Fc/2 fragment (2× closed lactone form of the payload), and one minor product (observed mass 26865 Da, approximately 30% of total Fc/2 fragment, calculated mass 26865 Da), corresponding to the conjugated Fc/2 fragment (1× closed lactone form and 1× open carboxylate form of the payload).

### Example 24. Preparation of ADC3 (DAR4)

To a solution of trastuzumab-(6-azidoGalNAc)₂ (333 μL, 11 mg, 33 mg/mL in PBS pH 7.4, trast-v1) was added 3 (350 μL, 2 mM solution in PG, 10 equiv. compared to IgG). The reaction was incubated for 18 h at RT followed by purification on an AKTA Purifier-10 (GE Healthcare) with a Superdex200 Increase 10/300 GL (GE Healthcare) column. Mass spectral analysis of the IdeS digested sample showed one major product (calculated mass 26732 Da, observed mass 26733 Da), corresponding to the conjugated ADC trast-v1-3 (ADC-3).

### Example 25. Preparation of ADC4a and ADC4b (DAR4 and DAR8)

Trastuzumab (290 mg, 18 mg/mL in formulation buffer+25 mM EDTA+25 mM tris pH 8.5, trast-v4) was incubated with TCEP (386 uL, 2.05 equiv μL, 10 mM in MQ) for 90 minutes. To the reaction deruxtecan (4, 1.6 mL, 6 equiv, 7.1 mM in DMA) was added followed by incubation for 90 minutes at room temperature. The reaction was subsequently quenched with an excess of N-acetyl cysteine and purified with TFF. Analysis on RP-HPLC showed the formation of the product trast-v4-4a with an average DAR of 3.94 (ADC4a).

ADC4b was prepared in a similar way by conjugation of compound 4 after reducing trast-v4 with 5-6 equivalents of TCEP. Analysis on RP-HPLC showed the formation of the product trast-v4-4b with an average DAR of 7.0 (ADC4b)

### Example 26. Conjugation of trastuzumab-(HC-L¹⁹⁶N mutant)-(6-N₃-GalNAc)₄ trast-v5 to 17b

To a solution of trastuzumab-(HC-L¹⁹⁶N mutant)-(6-N3-GalNAc)₄ (6.8 μL, 151 μg, 22.2 mg/ml in PBS pH 7.4, trast-v5) was added PBS pH 7.4 (0.7 μL) and 17b (2.5 μL, 8 mM solution in DMF, 20 equiv. compared to IgG). The reaction was incubated for 18 h at RT. Mass spectral analysis of the reduced sample showed three heavy chain products, corresponding to the unconjugated heavy chain (observed mass of 50317 Da, approximately 70% of total heavy chain products), the single conjugated heavy chain (observed mass of 52544 Da, approximately 25% of total heavy chain products), and the double conjugated heavy chain (observed mass of 54768 Da, approximately 5% of total heavy chain products).

### Example 27. Trastuzumab-(6-azidoGalNAc)₂ trast-v1 Conjugation with 1a

To a solution of trastuzumab-(6-azidoGalNAc)₂ (167 μL, 5 mg, 30 mg/mL in PBS pH 7.4, trast-v1) was added 1a (167 μL, 1.2 mM solution in PG, 6 equiv. compared to IgG). The reaction was incubated for 18 h at RT followed by purification on an AKTA Purifier-10 (GE Healthcare) with a Superdex200 Increase 10/300 GL (GE Healthcare) column. Mass spectral analysis of the IdeS digested sample showed one major product (calculated mass 26496 Da, observed mass 26498 Da), corresponding to the conjugated ADC trast-v1-1a.

### Example 28. Trastuzumab-(6-azidoGalNAc)₂ trast-v1 Conjugation with 2a

To a solution of trastuzumab-(6-azidoGalNAc)₂ (400 μL, 12 mg, 30 mg/mL in PBS pH 7.4, trast-v1) was added 2a (400 μL, 1.6 mM solution in PG, 8 equiv. compared to IgG). The reaction was incubated for 18 h at RT followed by purification on an AKTA Purifier-10 (GE Healthcare) with a Superdex200 Increase 10/300 GL (GE Healthcare) column. Mass spectral analysis of the IdeS digested sample showed one major product (calculated mass 26847 Da, observed mass 26847 Da), corresponding to the conjugated ADC trast-v1-2a.

### Example 29. Trastuzumab-(6-azidoGalNAc)₂ trast-v1 Conjugation with 21b

To a solution of trastuzumab-(6-azidoGalNAc)₂ (167 μL, 5 mg, 30 mg/mL in PBS pH 7.4) was added 21b (167 μL, 1.2 mM solution in PG, 6 equiv. compared to IgG). The reaction was incubated for 18 h at RT followed by purification on an AKTA Purifier-10 (GE Healthcare) with a Superdex200 Increase 10/300 GL (GE Healthcare) column. Mass spectral analysis of the IdeS digested sample showed one major product (calculated mass 26565 Da, observed mass 26567 Da), corresponding to the conjugated ADC trast-v1-21b.

### Example 30. Trastuzumab-(6-azidoGalNAc)₂ trast-v1 Conjugation with 1b

To a solution of trastuzumab-(6-azidoGalNAc)₂ (167 μL, 5 mg, 30 mg/mL in PBS pH 7.4, trast-v1) was added 1b (167 μL, 1.2 mM solution in PG, 6 equiv. compared to IgG). The reaction was incubated for 18 h at RT followed by purification on an AKTA Purifier-10 (GE Healthcare) with a Superdex200 Increase 10/300 GL (GE Healthcare) column. Mass spectral analysis of the IdeS digested sample showed one major product (calculated mass 26668 Da, observed mass 26671 Da), corresponding to the conjugated ADC trast-v1-1b.

### Example 31. Trastuzumab-(6-azidoGalNAc)₂ trast-v1 Conjugation with 25a

To a solution of trastuzumab-(6-azidoGalNAc)₂ (300 μL, 5 mg, 16.7 mg/mL in PBS pH 7.4, trast-v1) was added 50 μL sodium deoxycholate (110 mM in MQ) and 25a (150 μL, 1.33 mM solution in PG, 6 equiv. compared to IgG). The reaction was incubated for 18 h at RT followed by purification on an AKTA Purifier-10 (GE Healthcare) with a Superdex200 Increase 10/300 GL (GE Healthcare) column. RP-HPLC analysis of the DTT-reduced sample (see FIG. 13) showed a clear shift of the HC upon conjugation (RT_{HC-0}=8.7 min, RT_{HC-25a}=10.5 min), corresponding to an average drug to antibody ratio (DAR) of 3.66 indicating the formation of trast-v1-25a.

### Example 32. Trastuzumab-(6-azidoGalNAc)₂trast-v1 Conjugation with 26a

To a solution of trastuzumab-(6-azidoGalNAc)₂ (20.9 μL, 0.5 mg, 23.92 mg/mL in PBS pH 7.4, trast-v1) was added PBS pH 7.4 (9.1 μL), sodium deoxycholate (5 μL, 110 mM) and compound 26a (15 μL, 1.3 mM solution in PG, 6 equiv. compared to IgG). The reaction was incubated for 18 h at RT followed by buffer exchange to PBS pH 7.4 using centrifugal filters (Amicon Ultra 0.5 ml MWCO 10 kDa, Merck Millipore). Mass spectral analysis of the IdeS digested sample showed one major product (calculated mass 25504 Da, observed mass 25505 Da), corresponding to the conjugated ADC trast-v1-26a.

### Example 33. Trastuzumab-(6-azidoGalNAc)₂ trast-v1 Conjugation with 32a

To a solution of trastuzumab-(6-azidoGalNAc)₂ (12.54 μL, 0.3 mg, 23.92 mg/mL in PBS pH 7.4, trast-v1) was added PBS pH 7.4 (5.46 μL), sodium deoxycholate (3 μL, 110 mM) and compound 32a (9 μL, 0.8 mM solution in PG, 7 equiv. compared to IgG). The reaction was incubated for 18 h at RT followed by buffer exchange to PBS pH 7.4 using centrifugal filters (Amicon Ultra 0.5 ml MWCO 10 kDa, Merck Millipore). Mass spectral analysis of the IdeS-digested sample showed one major product (calculated mass 26753 Da, observed mass 26752 Da), corresponding to the conjugated ADC trast-v1-32a.

### Example 34. Trastuzumab-(6-azidoGalNAc)₂ trast-v1 Conjugation with 35a

To a solution of trastuzumab-(6-azidoGalNAc)₂ (12.54 μL, 0.3 mg, 23.92 mg/mL in PBS pH 7.4, trast-v1) was added sodium deoxycholate (3 μL, 110 mM) and compound 35a (15 μL, 1.2 mM solution in PG, 9 equiv. compared to IgG). The reaction was incubated for 18 h at RT followed by buffer exchange to PBS pH 7.4 using centrifugal filters (Amicon Ultra 0.5 ml MWCO 10 kDa, Merck Millipore). Mass spectral analysis of the IdeS digested sample showed one major product (calculated mass 26328 Da, observed mass 26329 Da), corresponding to the conjugated ADC trast-v1-35a.

### Example 35. Trastuzumab-(6-azidoGalNAc)₂ trast-v1 Conjugation with 31a

To a solution of trastuzumab-(6-azidoGalNAc)₂ (15 mL, 250 mg, 16.67 mg/mL in TBS pH 7.4, trast-v1) was added, 2.5 mL sodium deoxycholate (110 mM in MQ) and compound 31a (7.5 mL, 0.88 mM solution in PG, 4 equiv. compared to IgG). The reaction was incubated for 18 h at RT followed by purification on an AKTA Purifier-10 (GE Healthcare) with a Superdex200 Increase 10/300 GL (GE Healthcare) column. Mass spectral analysis of the IdeS digested sample showed one major product (calculated mass 26675 Da, observed mass 26672 Da), corresponding to the conjugated ADC trast-v1-31a.

### Example 36. Rituximab-(6-azidoGalNAc)₂ rit-v1 Conjugation with 39a

Enzymatic remodeling of rituximab to rituximab-(6-N₃-GalNAc)₂ was performed by incubating Rituximab (15 mg/mL) with EndoSH (1% w/w), as described in PCT/EP2017/052792, His-TnGalNAcT, as described in PCT/EP2016/059194 (5% w/w) and UDP 6-N3-GalNAc (25 eq compared to IgG), prepared according to PCT/EP2016/059194 in TBS containing 10 mM MnCl₂ for 16 hours at 30° C. Next, the functionalized IgG was purified using a HiTrap MabSelect Sure 5 mL column. After loading of the reaction mixture the column was washed with TBS+0.2% Triton and TBS. The IgG was eluted with 0.1 M glycine-HCl pH 2.7 and neutralized with 1 M Tris-HCl pH 8.8. After three times dialysis to PBS, the IgG was concentrated to 15-20 mg/mL using a Vivaspin Turbo 15 ultrafiltration unit (Sartorius).

Conjugation: Compound 39a (15 μl; 0.53 mM solution in PG, 4 equiv. compared to IgG) was added to a solution of rituximab-(6-azidoGalNAc)₂ (15 μl; 20 mg/ml in PBS pH 7.4, rit-v1). The reaction was incubated overnight at room temperature followed by buffer exchange to PBS pH 7.4 using centrifugal filters (Amicon Ultra 0.5 ml MWCO 10 kDa, Merck Millipore). Mass spectrometry analysis of the IdeS digested sample showed one major product (calculated mass 25619.6 Da, observed mass 25618.1 Da) corresponding to the conjugated ADC rit-v1-39a.

### Example 37. Trastuzumab-(6-azidoGalNAc)₂ trast-v1 Conjugation with 39a

Compound 39a (15 μl; 0.53 mM solution in PG, 4 equiv. compared to IgG) was added to a solution of trastuzumab-(6-azidoGalNAc)₂ (15 μl; 20 mg/ml in TBS pH 7.4, trast-v1). The reaction was incubated overnight at room temperature followed by buffer exchange to PBS pH 7.4 using centrifugal filters (Amicon Ultra 0.5 ml MWCO 10 kDa, Merck Millipore). Mass spectrometry analysis of the IdeS digested sample showed one major product (calculated mass 25653.8 Da, observed mass 25651.8 Da) corresponding to the conjugated ADC trast-v1-39a.

### Example 38. Rituximab-(6-azidoGalNAc)₂ rit-v1 Conjugation with 43a

Compound 43a (15 μl; 0.53 mM solution in PG, 4 equiv. compared to IgG) was added to a solution of rituximab-(6-azidoGalNAc)₂ (15 μl; 20 mg/ml in PBS pH 7.4, rit-v1). The reaction was incubated overnight at room temperature followed by buffer exchange to PBS pH 7.4 using centrifugal filters (Amicon Ultra 0.5 ml MWCO 10 kDa, Merck Millipore). Mass spectrometry analysis of the IdeS digested sample showed one major product (calculated mass 26532.5 Da, observed mass 26531.0 Da) corresponding to the conjugated ADC rit-v1-43a.

### Example 39. Trastuzumab-(6-azidoGalNAc)₂ trast-v1 Conjugation with 43a

Compound 43a (15 μl; 0.53 mM solution in PG, 4 equiv. compared to IgG) was added to a solution of trastuzumab-(6-azidoGalNAc)₂ (15 μl; 20 mg/ml in TBS pH 7.4, trast-v1). The reaction was incubated overnight at room temperature followed by buffer exchange to PBS pH 7.4 using centrifugal filters (Amicon Ultra 0.5 ml MWCO 10 kDa, Merck Millipore). Mass spectrometry analysis of the IdeS digested sample showed one major product (calculated mass 26566.7 Da, observed mass 26564.5 Da) corresponding to the conjugated ADC trast-v1-43a.

### Example 40. Rituximab-(6-azidoGalNAc)₂ rit-v1 Conjugation with 49a

Compound 49a (15 μl; 0.53 mM solution in PG, 4 equiv. compared to IgG) was added to a solution of rituximab-(6-azidoGalNAc)₂ (15 μl; 20 mg/ml in PBS pH 7.4, rit-v1). The reaction was incubated overnight at room temperature followed by buffer exchange to PBS pH 7.4 using centrifugal filters (Amicon Ultra 0.5 ml MWCO 10 kDa, Merck Millipore). Mass spectrometry analysis of the IdeS digested sample showed one major product (calculated mass 26750.7 Da, observed mass 26751.1 Da) corresponding to the conjugated ADC rit-v1-49a.

### Example 41. Trastuzumab-(6-azidoGalNAc)₂trast-v1 Conjugation with 49a

Compound 49a (15 μl; 0.53 mM solution in PG, 4 equiv. compared to IgG) was added to a solution of trastuzumab-(6-azidoGalNAc)₂ (15 μl; 20 mg/ml in TBS pH 7.4, trast-v1). The reaction was incubated overnight at room temperature followed by buffer exchange to PBS pH 7.4 using centrifugal filters (Amicon Ultra 0.5 ml MWCO 10 kDa, Merck Millipore). Mass spectrometry analysis of the IdeS digested sample showed one major product (calculated mass 26784.9 Da, observed mass 26783.8 Da) corresponding to the conjugated ADC trast-v1-49a.

### Example 42. Rituximab-(6-azidoGalNAc)₂ rit-v1 Conjugation with 52a

Compound 52a (15 μl; 0.53 mM solution in PG, 4 equiv. compared to IgG) was added to a solution of rituximab-(6-azidoGalNAc)₂ (15 μl; 20 mg/ml in PBS pH 7.4, rit-v1). The reaction was incubated overnight at room temperature followed by buffer exchange to PBS pH 7.4 using centrifugal filters (Amicon Ultra 0.5 ml MWCO 10 kDa, Merck Millipore). Mass spectrometry analysis of the IdeS digested sample showed one major product (calculated mass 26381.4 Da, observed mass 26381.9 Da) corresponding to the conjugated ADC rit-v1-52a.

### Example 43. Trastuzumab-(6-azidoGalNAc)₂trast-v1 Conjugation with 52a

Compound 52a (15 μl; 0.53 mM solution in PG, 4 equiv. compared to IgG) was added to a solution of trastuzumab-(6-azidoGalNAc)₂ (15 μl; 20 mg/ml in TBS pH 7.4 trast-v1). The reaction was incubated overnight at room temperature followed by buffer exchange to PBS pH 7.4 using centrifugal filters (Amicon Ultra 0.5 ml MWCO 10 kDa, Merck Millipore). Mass spectrometry analysis of the IdeS digested sample showed one major product (calculated mass 26415.6, observed mass 26414.2 Da) corresponding to the conjugated ADC trast-v1-52a.

### Example 44. Rituximab-(6-azidoGalNAc)₂ rit-v1 Conjugation with 1a

To a solution of rituximab-(6-azidoGalNAc)₂ (12.24 μL, 0.3 mg, 24.5 mg/ml in PBS pH 7.4, rit-v1) was added sodium deoxycholate (3 μL, 110 mM) and compound 1a (9 μl, 1.33 mM solution in PG, 6 equiv. compared to IgG). The reaction was incubated for 18 h at RT followed by buffer exchange to PBS pH 7.4 using centrifugal filters (Amicon Ultra 0.5 ml MWCO 10 kDa, Merck Millipore). Mass spectral analysis of the IdeS digested sample showed product formation (calculated mass 26463 Da, observed mass 26461 Da), corresponding to the conjugated ADC rit-v1-1a.

### Example 45. Rituximab-(6-azidoGalNAc)₂ rit-v1 Conjugation with 32a

To a solution of rituximab-(6-azidoGalNAc)₂ (12.24 μL, 0.3 mg, 24.5 mg/ml in PBS pH 7.4, rit-v1) was added sodium deoxycholate (3 μL, 110 mM) and compound 32a (9 μl, 0.89 mM solution in PG, 4 equiv. compared to IgG). The reaction was incubated for 18 h at RT followed by buffer exchange to PBS pH 7.4 using centrifugal filters (Amicon Ultra 0.5 ml MWCO 10 kDa, Merck Millipore). Mass spectral analysis of the IdeS digested sample showed product formation (calculated mass 26721 Da, observed mass 26720 Da), corresponding to the conjugated ADC rit-v1-32a.

### Example 46. Rituximab-(6-azidoGalNAc)₂ rit-v1 Conjugation with 31a

To a solution of rituximab-(6-azidoGalNAc)₂ (12.24 μL, 0.3 mg, 24.5 mg/ml in PBS pH 7.4, rit-v1) was added sodium deoxycholate (3 μL, 110 mM) and compound 31a (9 μL, 0.89 mM solution in PG, 4 equiv. compared to IgG). The reaction was incubated for 18 h at RT followed by buffer exchange to PBS pH 7.4 using centrifugal filters (Amicon Ultra 0.5 ml MWCO 10 kDa, Merck Millipore). Mass spectral analysis of the IdeS digested sample showed product formation (calculated mass 26642 Da, observed mass 26640 Da), corresponding to the conjugated ADC rit-v1-31a.

### Example 47. Enzymatic Remodeling of Trastuzumab to trastuzumab-(GalNProSSMe)₂ (Trast-v2b)

Trastuzumab (5 mg, 22.7 mg/mL) was incubated with EndoSH, described in PCT/EP2017/052792 (1% w/w), for 1h followed by the addition TnGalNAcT (expressed in CHO), (10% w/w) and UDP-GalNProSSMe, (40 eq. compared to IgG) in 10 mM MnCl₂ and TBS for 16 hours at 30° C. After addition of the components the final concentration of trastuzumab is 12.5 mg/ml. The functionalized IgG was purified using a protA column (5 mL, MabSelect Sure, Cytiva). After loading of the reaction mixture the column was washed with TBS. The IgG was eluted with 0.1 M NaOAc pH 3.5 and neutralized with 2.5 M Tris-HCl pH 7.2. After three times dialysis to PBS the functionalized trastuzumab was concentrated to 17.4 mg/mL using a Vivaspin Turbo 4 ultrafiltration unit (Sartorius). Mass spectral analysis of a sample after IdeS treatment showed one major Fc/2 product (observed mass 24430 Da) corresponding to the expected product (trast-v2b).

### Example 48. Conjugation of trastuzumab S239C Mutant trast-v3 with maleimide-exatecan Variants 54a, 57b, 59a or 60a

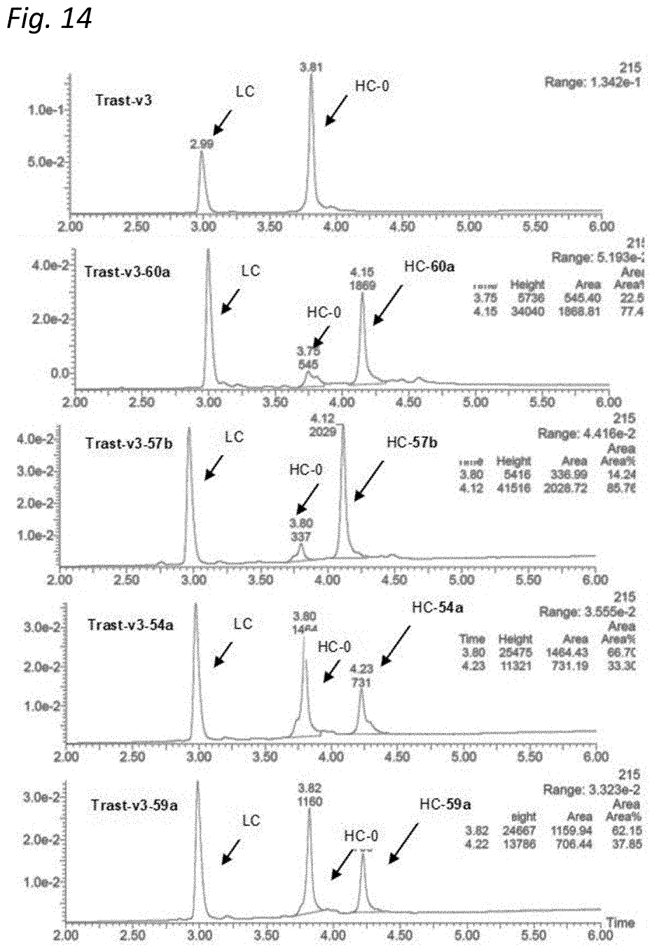

Trastuzumab S239C mutant (transient expressed in CHO by Evitria, heavy chain mutation S239C) (1 mg, 10 mg/mL in PBS+10 mM EDTA, trast-v3) was incubated with TCEP (6.5 μL, 10 mM in MQ) for two hours at 37° C. The reduced antibody was spin-filtered with PBS+10 mM EDTA using centrifugal filters (Amicon Ultra-0.5 mL MWCO 10 kDa, Merck Millipore) and subsequently diluted to 100 μL. Subsequently DHA (6.5 μL, 10 mM in MQ) was added and the reaction was incubated for three hours at room temperature. The reaction was divided into four portions (each 20 μL, 0.2 mg antibody) and to each portion a maleimide-exatecan variant (54a, 57b, 59a or 60a) (2.3 μL, 5 mM in DMF) was added followed by incubation for one hour at room temperature. The conjugates were spin-filtered to PBS using centrifugal filters (Amicon Ultra-0.5 mL MWCO 10 kDa, Merck Millipore) and subsequently analyzed by RP-UPLC (see FIG. 14).

| Conjugate | DAR |
|---|---|
| Trast-v3-60a | 1.55 |
| Trast-v3-57b | 1.72 |
| Trast-v3-54a | 0.66 |
| Trast-v3-59a | 0.76 |

### Example 49. Conjugation of trastuzumab GalProSH trast-v2 with maleimide-exatecan Variants 60a

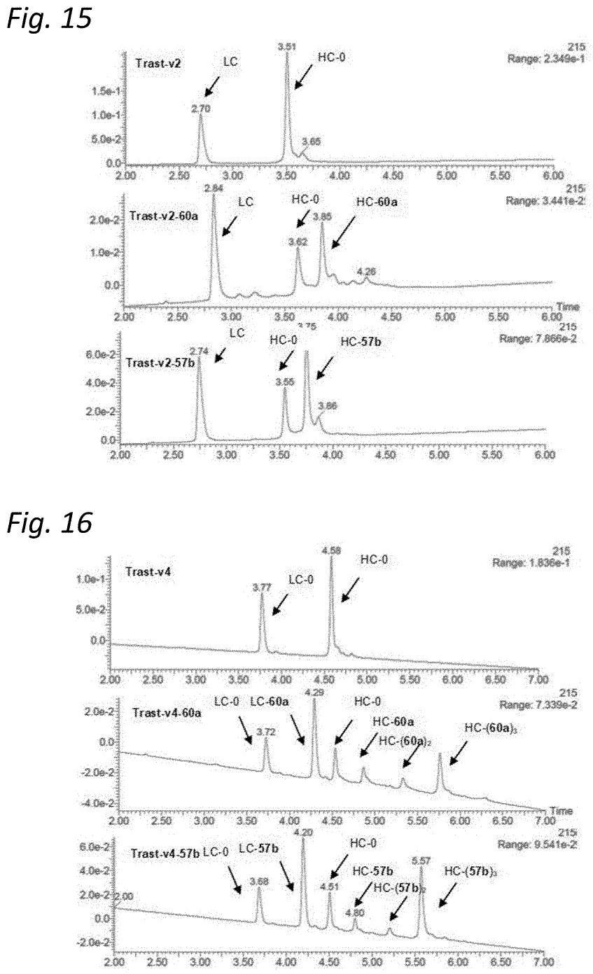

Trastuzumab GalProSSMe (0.5 mg, 10 mg/mL in PBS+10 mM EDTA, trast-v2b) was incubated with TCEP (3.3 μL, 10 mM in MQ) for two hours at 37° C. The reduced antibody was spin-filtered with PBS+10 mM EDTA using centrifugal filters (Amicon Ultra-0.5 mL MWCO 10 kDa, Merck Millipore). Subsequently dehydroascorbic acid (DHA 3.3 μL, 10 mM in MQ) was added and the reaction was incubated for three hours at room temperature. To a portion of the reaction (10 μL, 0.1 mg antibody) was added maleimide-exatecan 60a (1.3 μL, 5 mM in DMF) followed by incubation for one hour at room temperature. The conjugate was spin-filtered to PBS using centrifugal filters (Amicon Ultra-0.5 mL MWCO 10 kDa, Merck Millipore) and subsequently analyzed on RP-UPLC. RP-UPLC analysis of DTT-treated conjugate, showed the conversion into the conjugate trast-v2-60a with a DAR of 1.15 (see FIG. 15).

### Example 50. Conjugation of trastuzumab GalProSH trast-v2 with maleimide-exatecan Variants 57b

Trastuzumab GalProSSMe (0.5 mg, 10 mg/mL in PBS+10 mM EDTA, trast-v2b) was incubated with TCEP (3.3 μL, 10 mM in MQ) for two hours at 37° C. The reduced antibody was spin-filtered with PBS+10 mM EDTA using centrifugal filters (Amicon Ultra-0.5 mL MWCO 10 kDa, Merck Millipore). Subsequently DHA (3.3 μL, 10 mM in MQ) was added and the reaction was incubated for three hours at room temperature. To a portion of the reaction (10 μL, 0.1 mg antibody) was added maleimide-exatecan 57b (1.3 μL, 5 mM in DMF) followed by incubation for one hour at room temperature. The conjugate was spin-filtered to PBS using centrifugal filters (Amicon Ultra-0.5 mL MWCO 10 kDa, Merck Millipore) and subsequently analyzed by RP-UPLC. RP-UPLC analysis of DTT-treated conjugate showed the conversion into the conjugate trast-v2-57b with a DAR of 1.37 (see FIG. 15).

### General Experimentals: Interchain Maleimide Conjugation after TCEP Reduction Step

General experimental A—Conjugation with 3.5 equiv. TCEP: mAb-v4 (10 mg/mL in PBS+10 mM EDTA) was incubated with TCEP (3.5 equiv, 10 mM in MQ) for 1 hour at 37° C. To a portion of the reaction (20 μL, 0.2 mg antibody) was added a maleimide-exatecan (2.3 μL, 5 mM in DMF) followed by incubation for 1 hour at room temperature. The conjugate was spin-filtered to PBS using centrifugal filters (Amicon Ultra-0.5 mL MWCO 10 kDa, Merck Millipore) and subsequently analyzed on RP-UPLC. The DAR was calculated according to the formula below.

General experimental B—Conjugation with 7 equiv. TCEP: mAb-v4 (10 mg/mL in PBS+10 mM EDTA) was incubated with TCEP (7 equiv, 10 mM in MQ) for 1 hour at 37° C. To a portion of the reaction (20 μL, 0.2 mg antibody) was added a maleimide-exatecan (10-35 equiv, in DMF) followed by incubation for 1 hour at room temperature. The conjugate was spin-filtered to PBS using centrifugal filters (Amicon Ultra-0.5 mL MWCO 10 kDa, Merck Millipore) and subsequently analyzed on RP-UPLC. The DAR was calculated according to the formula below.

Formula for average DAR calculation:

$$avg DAR = 2 x \frac{peak area HC 1}{total peak area HC peaks} \text{} + 4 x \frac{peak area HC 2}{total peak area HC peaks} \text{} + 6 \text{} x \text{} \frac{peak area HC 3}{total peak area HC peaks} \text{} + \text{} 2 x \frac{peak area LC 1}{total peak area LC peaks}$$

### Example 51. Conjugation of trastuzumab trast-v4 with maleimide-exatecan 60a

According to the general experimental A trastuzumab was conjugated to maleimide-exatecan 60a. RP-UPLC analysis of DTT treated conjugate, showed the conversion into the conjugate trast-v4-60a with a DAR of 4.6 (see FIG. 16).

### Example 52. Conjugation of trastuzumab trast-v4 with maleimide-exatecan 57b

According to the general experimental A trastuzumab was conjugated to maleimide-exatecan 57b. RP-UPLC analysis of DTT treated conjugate, showed the conversion into the conjugate trast-v4-57b with a DAR of 5.6 (see FIG. 16).

### Example 53. Conjugation of rituximab rit-v4 with maleimide-exatecan 60a

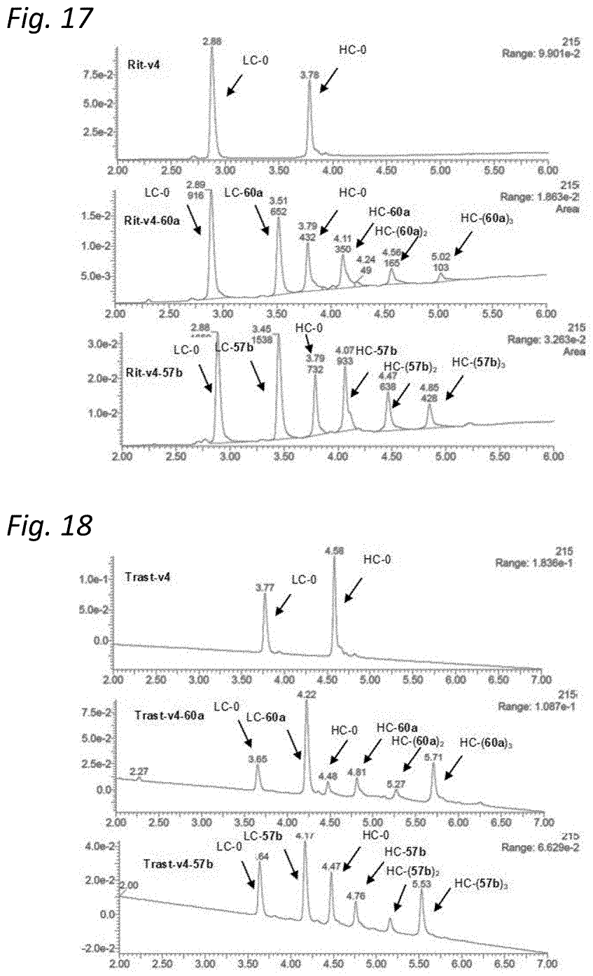

According to the general experimental A rituximab was conjugated to maleimide-exatecan 60a. RP-UPLC analysis of DTT treated conjugate, showed the conversion into the conjugate rit-v4-60a with a DAR of 2.5 (see FIG. 17).

### Example 54. Conjugation of rituximab rit-v4 with maleimide-exatecan 57b

According to the general experimental A rituximab was conjugated to maleimide-exatecan 57b. RP-UPLC analysis of DTT treated conjugate, showed the conversion into the conjugate rit-v4-57b with a DAR of 3.4 (see FIG. 17).

### Example 55. Conjugation of trastuzumab trast-v4 with maleimide-exatecan 59a

According to the general experimental A trastuzumab was conjugated to maleimide-exatecan 59a. RP-UPLC analysis of DTT treated conjugate, showed the conversion into the conjugate trast-v4-59a with a DAR of 2.2.

### Example 56. Conjugation of rituximab rit-v4 with maleimide-exatecan 59a

According to the general experimental A rituximab was conjugated to maleimide-exatecan 59a. RP-UPLC analysis of DTT treated conjugate, showed the conversion into the conjugate rit-v4-59a with a DAR of 3.6.

### Example 57. Conjugation of trastuzumab trast-v4 with maleimide-exatecan 60a

According to the general experimental B trastuzumab was conjugated to maleimide-exatecan 60a. RP-UPLC analysis of DTT treated conjugate, showed the conversion into the conjugate trast-v4-60a with a DAR of 5.7 (see FIG. 18).

### Example 58. Conjugation of trastuzumab trast-v4 with maleimide-exatecan 57b

According to the general experimental B trastuzumab was conjugated to maleimide-exatecan 57b. RP-UPLC analysis of DTT treated conjugate, showed the conversion into the conjugate trast-v4-57b with a DAR of 6.2 (see FIG. 18).

### Example 59. Conjugation of rituximab rit-v4 with maleimide-exatecan 60a

According to the general experimental B rituximab was conjugated to maleimide-exatecan 60a. RP-UPLC analysis of DTT treated conjugate, showed the conversion into the conjugate rit-v4-60a with a DAR of 5.8.

### Example 60. Conjugation of trastuzumab trast-v4 with maleimide-exatecan 59a

According to the general experimental B trastuzumab was conjugated to maleimide-exatecan 59a. RP-UPLC analysis of DTT treated conjugate, showed the conversion into the conjugate trast-v4-59a with a DAR of 2.6.

### Example 61. Conjugation of rituximab rit-v4 with maleimide-exatecan 59a

According to the general experimental B rituximab was conjugated to maleimide-exatecan 59a. RP-UPLC analysis of DTT treated conjugate, showed the conversion into the conjugate rit-v4-59a with a DAR of 4.4.

### Example 62. Aggregation Study

ADCs (1 mg/mL in PBS pH 7.4) were incubated at 37° C. Aggregation level was measured after 0, 1, 4, 7, 14 and 21 days using an Agilent 1100 series HPLC (Hewlett Packard). The sample (3 μL, 1 mg/mL) was injected with 0.86 mL/min onto a Xbridge BEH200 Å column (3.5 μM, 7.8×300 mm, Waters). Isocratic elution using 0.1 M sodium phosphate buffer pH 6.9 (NaH₂PO₄/Na₂HPO₄) was performed for 16 minutes. Aggregation levels are shown in Table 1.

TABLE 1
Aggregation levels (%) of ADC1a and ADC2
(DAR4) incubated in PBS pH 7.4 at 37° C.

| | Incubation time (days) | | | | |
|---|---|---|---|---|---|
| Conjugate | 0 | 2 | 7 | 14 | 21 |
| ADC1a (DAR4) | 0.0% | 0.0% | 1.0% | 1.1% | 1.1% |
| ADC2 (DAR4) | 0.0% | 0.8% | 0.0% | 0.0% | 0.0% |

### Example 63: Analytical HIC

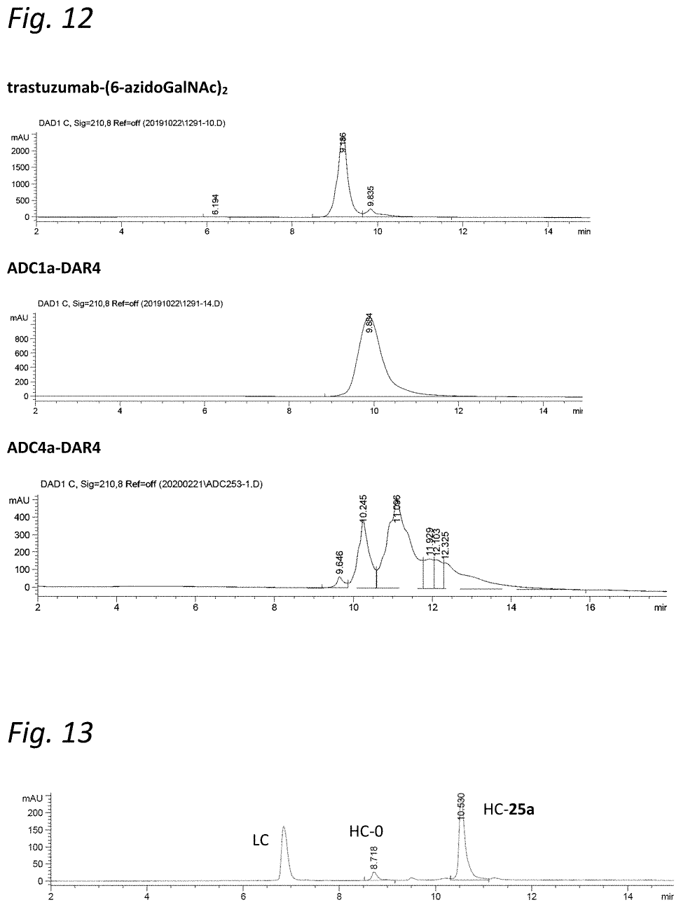

HIC analysis was performed on an Agilent 1100 series HPLC (Hewlett Packard). The sample (3 μL 1 mg/mL in PBS) was injected with 0.8 mL/min onto a TSKgel® Butyl-NPR HPLC column (3.5 cm×4.6 mm, 2.5 μm, Tosoh Bioscience). A linear gradient was applied in 13 minutes from 2 M ammonium sulfate in 50 mM potassium phosphate pH 6.0 to 20% isopropanol in 50 mM potassium phosphate pH 6.0. Results of HIC analyses is shown in FIG. 12.

### Example 64: BT-474 Efficacy Studies

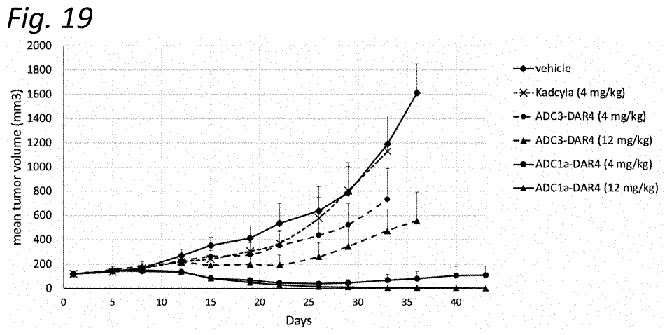

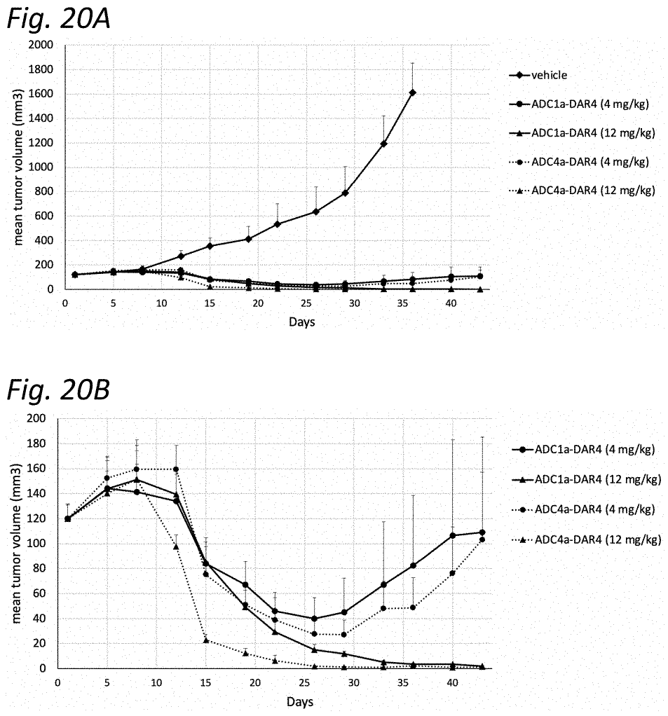

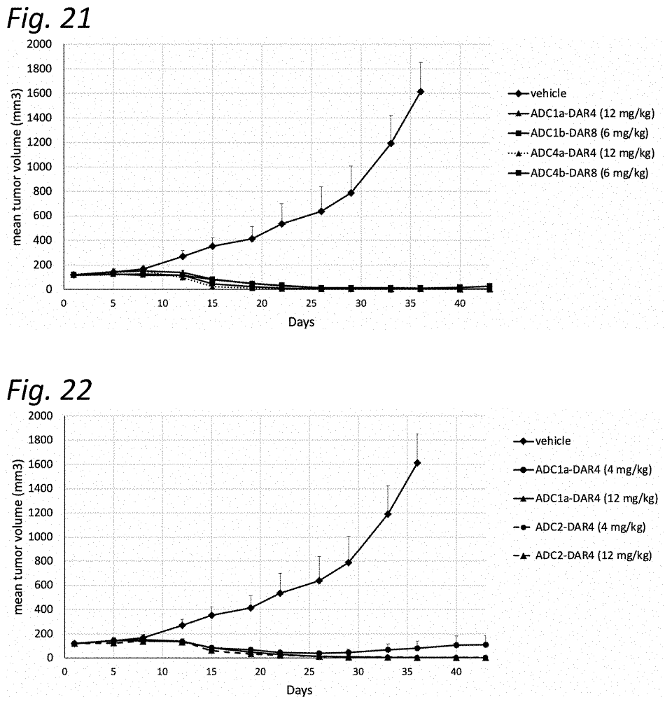

CR female CB.17 SCID mice, 8- to 12-week old at the beginning of the experimental phase, obtained from Charles River Laboratories, USA) were injected with 1×107 BT-474 tumour cells in a 50% Matrigel subcutaneous in the flank (BT-474 cell xenograft model). When the tumour volume was in the range of 100-150 mm³, groups of seven mice were injected i.v. with a single dose at day 1 with test items and dose levels indicated below. Tumours were measured twice weekly for a period of 43 days. The results on tumour volume (mean) are depicted in FIGS. 19-22.

TABLE 2
Overview of test items and dose levels included in in vivo study

| Conjugate | Dose levels (mg/kg) |
|---|---|
| ADC1a-DAR4 | 4 |
| | 12 |
| ADC1a-DAR8 | 6 |
| ADC2 (DAR4) | 4 |
| | 12 |
| ADC3 (DAR4) | 4 |
| | 12 |
| ADC4a (DAR4) | 4 |
| | 12 |
| ADC4b (DAR8) | 6 |
| Kadcyla | 4 |
| Vehicle | — |

1. An antibody-drug conjugate, having structure (1) 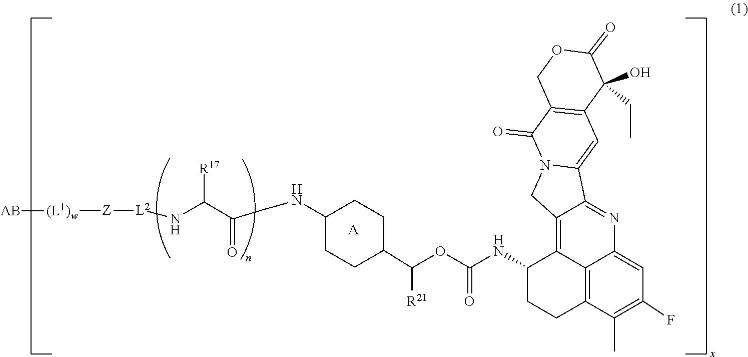
wherein:
AB is an antibody;
L¹ and L² are linkers;
w is 0 or 1;
Z is a connecting group obtained by a metal-free click reaction or by thiol ligation;
each R¹⁷ is individually an amino acid side chain;
n is an integer in the range of 1-5;
A is a 5- or 6-membered aromatic or heteroaromatic ring;
x is an integer in the range of 1-8;
R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups.

2. The antibody-drug conjugate according to claim 1, wherein L² has structure (2) 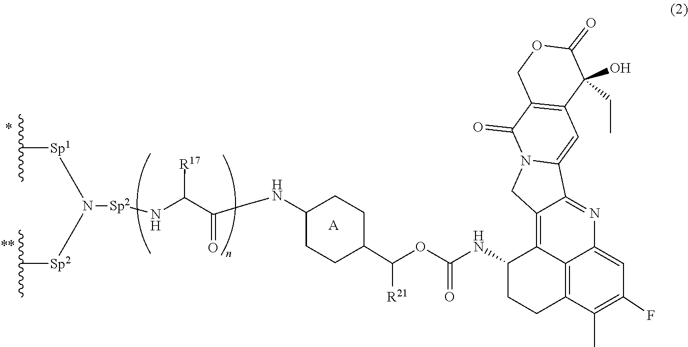
wherein
the wavy bond labeled with * is connected to Z and the wavy bond labeled with ** is connected to NH;
Sp¹ and Sp² are each individually spacer moieties;
n, A, R¹⁷ and R²¹ are as defined as in claim 1.

3. The antibody-drug conjugate according to claim 2, wherein each occurrence of Sp² is the same, each occurrence of (NH—CR¹⁷—CO)ₙ is the same, each occurrence of A is the same or each occurrence of R²¹ is the same.

4. The antibody-drug conjugate according to claim 1, wherein L², comprises a sulfamide group according to structure (3) 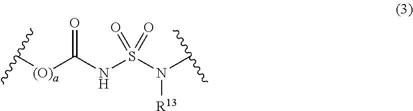
wherein
a=0 or 1, and
R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is a second occurrence of C(O)X connected to N via a spacer moiety.

5. The antibody-drug conjugate according to claim 4, wherein Sp¹ comprises two groups of formula (3).

6. The antibody-drug conjugate according to claim 1, wherein each occurrence of the peptide (NH—CR¹⁷—CO)ₙ is selected from Val-Cit, Val-Ala, Val-Lys, Val-Arg, AcLys-Val-Cit, AcLys-Val-Ala, Glu-Val-Ala, Asp-Val-Ala, Phe-Cit, Phe-Ala, Phe-Lys, Phe-Arg, Ala-Lys, Leu-Cit, Ile-Cit, Trp-Cit, Ala-Ala-Asn, Ala-Asn and Lys.

7. The antibody-drug conjugate according to claim 1, wherein Z has a structure selected from (Z1)-(Z8) and (Z11)-(Z23): 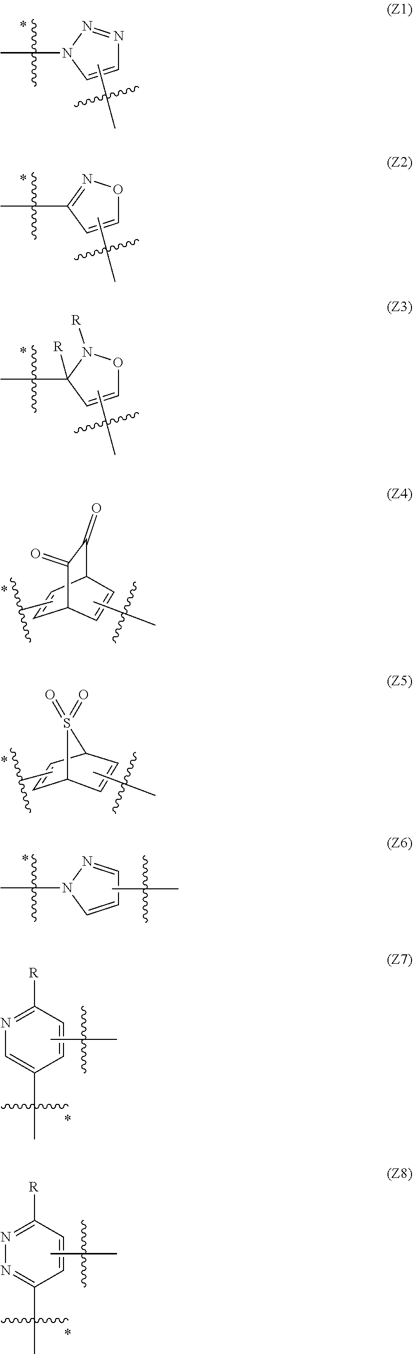 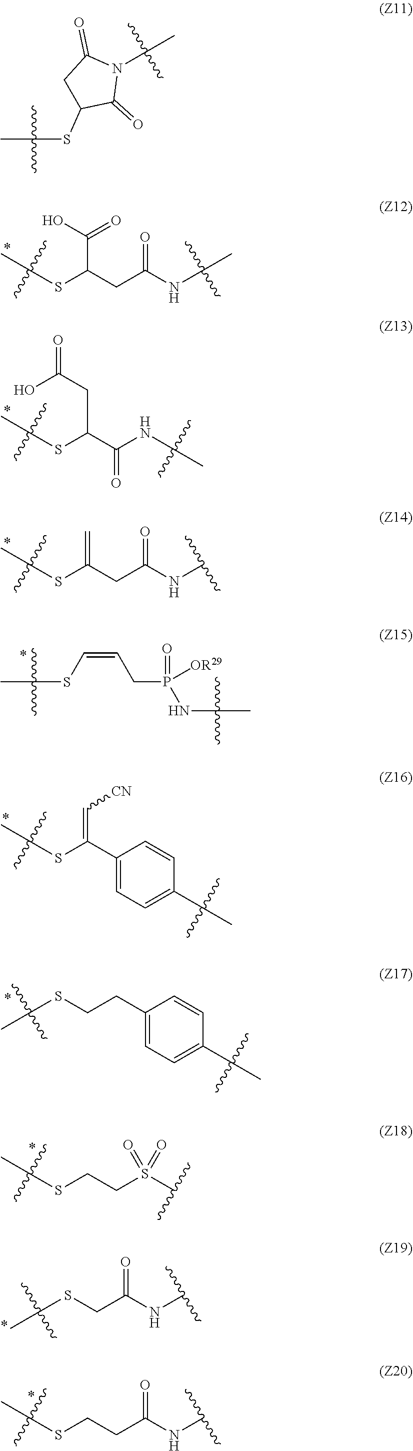 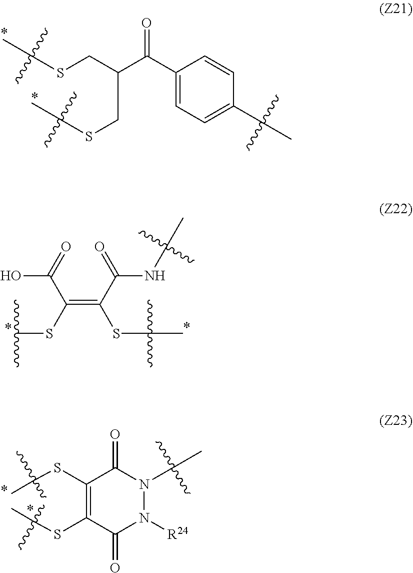
wherein:
the wavy bond labelled with an * is connected to AB, optionally via L¹, and the other wavy bond to L²;
functional groups R in (Z3), (Z7) and (Z8) is selected from hydrogen, C₁-C₂₄ alkyl groups, C₂-C₂₄ acyl groups, C₃-C₂₄ cycloalkyl groups, C₂-C₂₄ (hetero)aryl groups, C₃-C₂₄ alkyl(hetero)aryl groups, C₃-C₂₄ (hetero)arylalkyl groups and C₁-C₂₄ sulfonyl groups, each of which may optionally be substituted and optionally be interrupted by one or more heteroatoms selected from O, S and NR³² wherein R³² is independently selected from the group consisting of hydrogen and C₁-C₄ alkyl groups;
R²⁴ is H or C₁₋₁₂ alkyl;
R²⁹ is C₁₋₁₂ alkyl.

8. The antibody-drug conjugate according to claim 1, which has structure (if) or (1b): 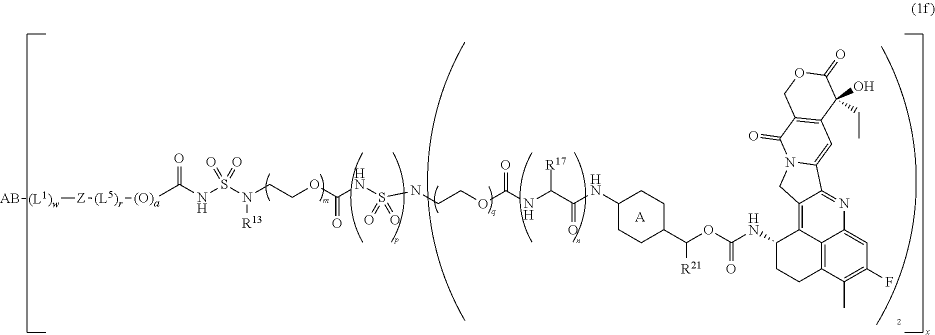
wherein:
AB, L¹, Z, A, R²¹, n, R¹⁷ and x are as defined in claim 1;
a=0 or 1;
R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is a second occurrence of C(O)X connected to N via a spacer moiety;
L⁵ is a linker;
r is 0 or 1,
m is an integer in the range of 1-10;
q is an integer in the range of 0-10;
p is 0 or 1; 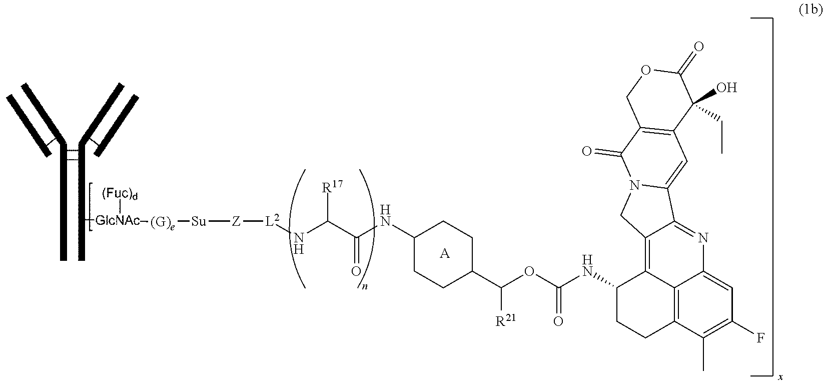
wherein:
Z, L², R¹⁷, A, R²¹, n and x are as defined in claim 1;
e is an integer in the range of 0-20;
Su is a monosaccharide;
G is a monosaccharide moiety;
GlcNAc is an N-acetylglucosamine moiety;
Fuc is a fucose moiety;
d is 0 or 1.

9. The antibody-drug conjugate according to claim 8, wherein the antibody-drug conjugate has structure (1h): 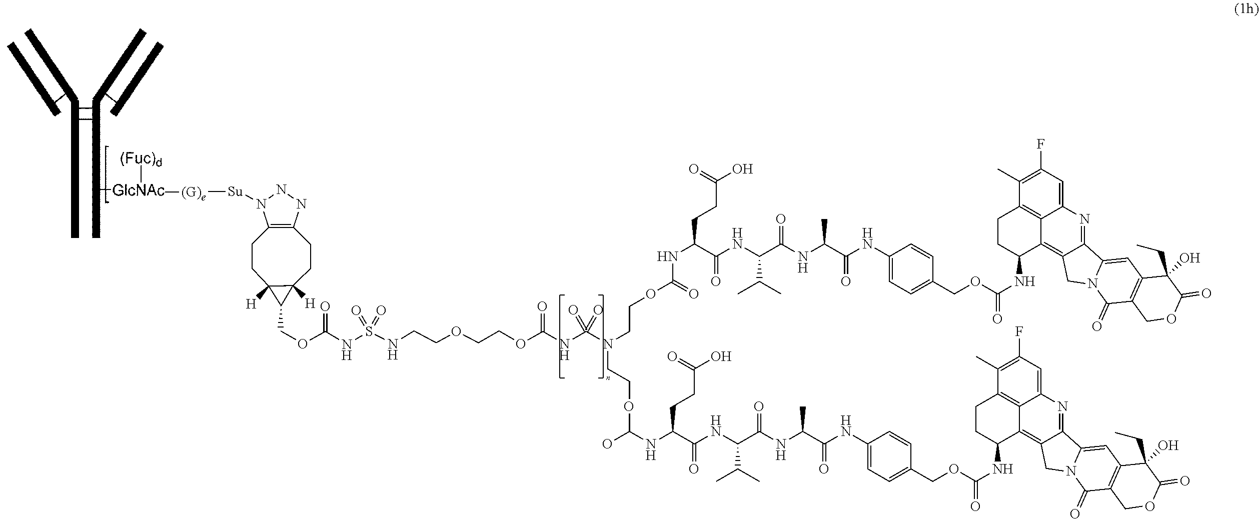
wherein:
e, Su, G, GlcNAc, Fuc and d are as defined as in claim 8;
n is 0 or 1.

10. A process for the synthesis of the antibody-drug conjugate according to claim 1, comprising reacting
(i) a modified antibody of structure AB-((L¹)_{w}-F)ₓ, wherein:
AB is an antibody;
L¹ is a linker;
w is 0 or 1;
F is a click probe capable of reacting with Q in a metal-free click reaction or a thiol or precursor thereof;
x is an integer in the range of 1-8;
with
(ii) a linker-drug construct according to structure (5): 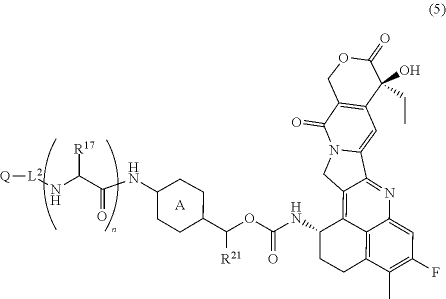
wherein:
L² is a linker;
Q is a click probe capable of performing a metal-free click reaction or a thiol-reactive probe;
each R¹⁷ is individually an amino acid side chain;
n is an integer in the range of 1-5;
A is a 5- or 6-membered aromatic or heteroaromatic ring;
x is an integer in the range of 1-8;
R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups,
to form an antibody-drug conjugate wherein the drug is covalently attached to the antibody via connecting group Z that is formed by a metal-free click reaction between Q and F or by thiol ligation between Q and F.

11. The process according to claim 10, wherein the click probe Q comprises a cyclic alkyne moiety or a cyclic alkene moiety.

12. The process according to claim 11, wherein:
(a) the click probe Q is selected from the group consisting of (Q22)-(Q36): 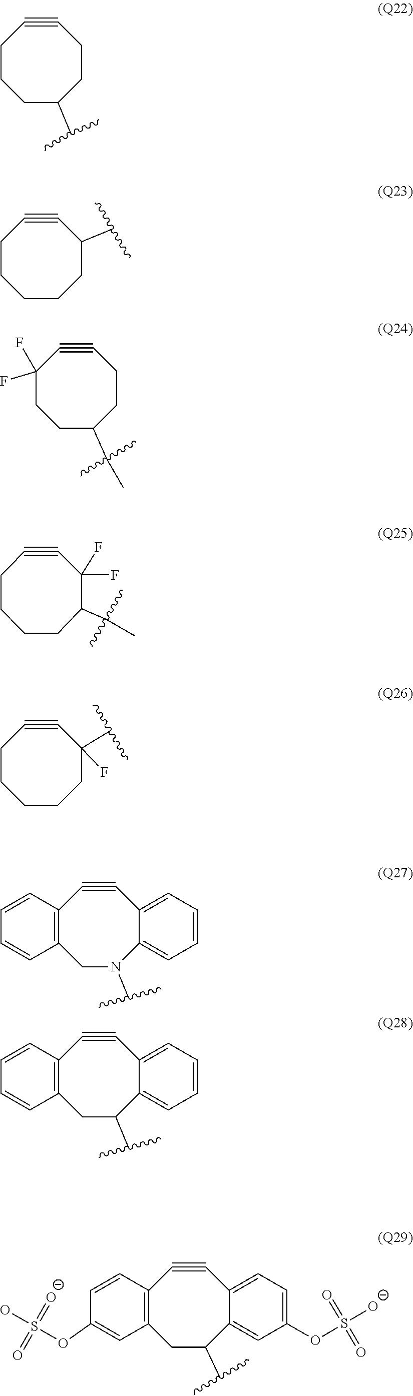 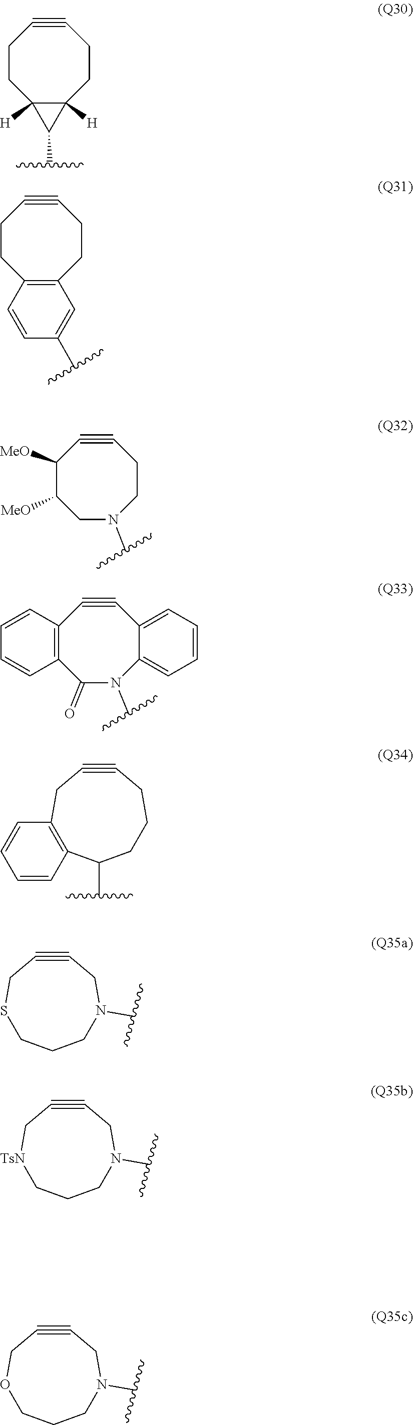 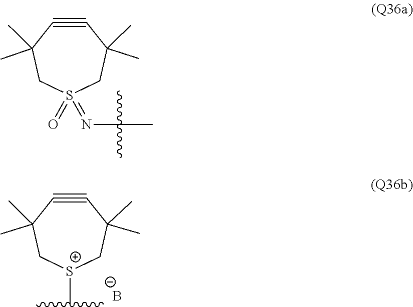
wherein B is a pharmaceutically acceptable anion;
or wherein the (hetero)cycloalkynyl moiety Q is according to structure (Q37): 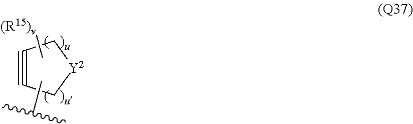
wherein:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, 07-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, 07-C₂₄ alkyl(hetero)aryl groups and C₇-C₂₄ (hetero)arylalkyl groups;
Y² is C(R³¹)₂, O, S or NR³¹, wherein each R³¹ individually is R¹⁵ or a second occurrence of the exatecan payload connected via a spacer moiety;
u is 0, 1, 2, 3, 4 or 5;
u′ is 0, 1, 2, 3, 4 or 5, wherein u+u′=4, 5, 6, 7 or 8;
v=an integer in the range 8-16.

13. The process according to claim 12, wherein the cyclooctynyl moiety Q is according to structure (Q38): 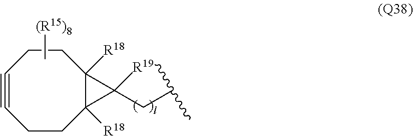
wherein
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, 07-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, 07-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of the exatecan payload connected via a spacer moiety; and
I is an integer in the range 0 to 10;
or wherein the (hetero)cyclooctynyl moiety Q is according to structure (Q39): 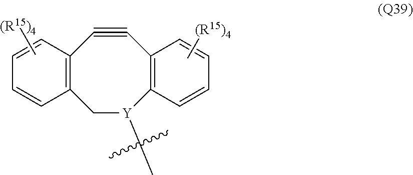
wherein
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵;
or wherein the heterocycloheptynyl moiety Q is according to structure (Q36a): 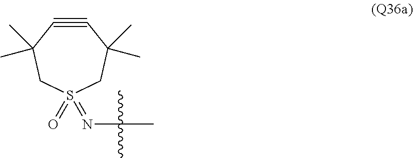
or (b) wherein the click probe Q is selected from the group consisting of, optionally substituted, (hetero)cyclopropenyl group, (hetero)cyclobutenyl group, trans-(hetero)cycloheptenyl group, trans-(hetero)cyclooctenyl group, trans-(hetero)cyclononenyl group or trans-(hetero)cyclodecynyl group.

14. The process according to claim 13, wherein click probe Q is selected from the group consisting of (Q40)-(Q50): 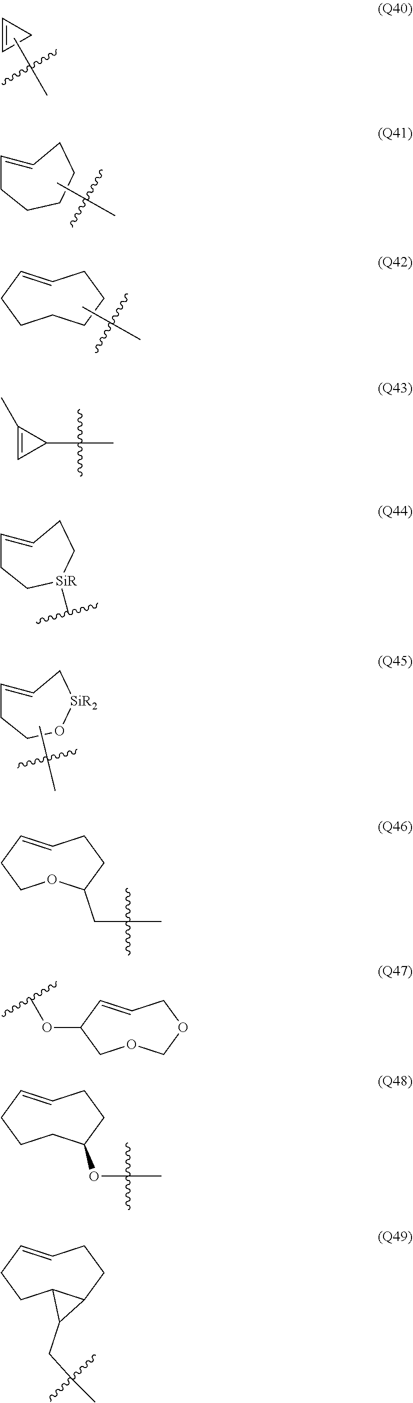 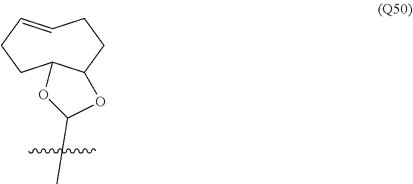 [SMILES: *C1OC2CC/C=C/CCC2O1]
wherein the R group(s) on Si in (Q44) and (Q45) is alkyl or aryl.

15. The process according to claim 10, wherein the thiol-reactive probe Q is selected from the group consisting of (Q51)-(Q65): 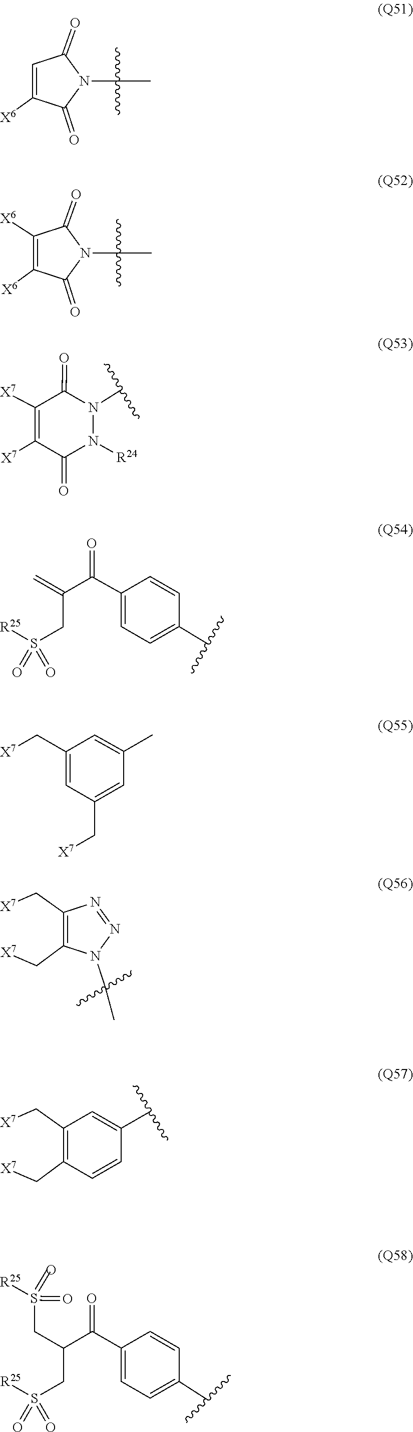 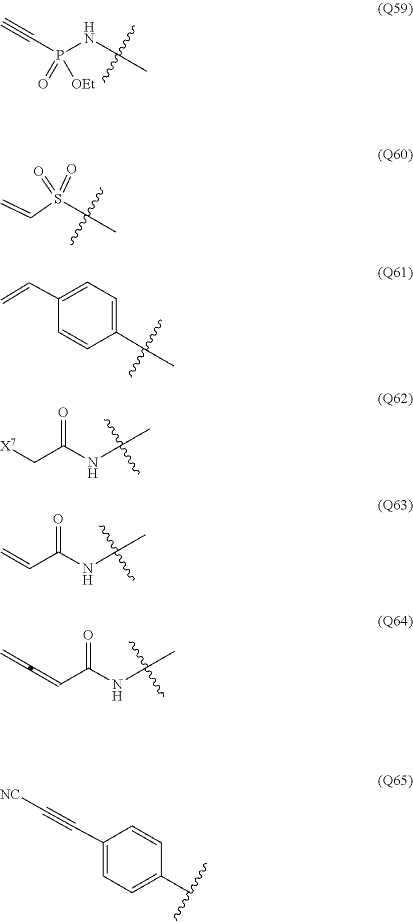
wherein:
X⁶ is H, halogen, PhS, MeS;
X⁷ is halogen, PhS, MeS;
R²⁴ is H or C₁₋₁₂ alkyl;
R²⁵ is H, C₁₋₁₂ alkyl, C₁₋₁₂ aryl, C₁₋₁₂ alkaryl or C₁₋₁₂ aralkyl;
wherein the aromatic ring of (Q55) and (Q57) may optionally be a heteroaromatic ring.

16. The process according to claim 15, wherein the heteroaromatic ring is a phenyl or pyridine ring

17. The process according to claim 10, wherein:
click probe F is selected from the group consisting of azide, tetrazine, triazine, nitrone, nitrile oxide, nitrile imine, diazo compound, ortho-quinone, dioxothiophene and sydnone; or
F is a thiol; and/or wherein the thiol ligation is a Michael addition or a nucleophilic substitution.

18. The process according to claim 10, wherein AB-((L¹)_{w}-F)ₓ is according to structure (6): 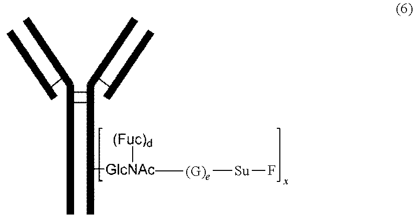
wherein:
e is an integer in the range of 0-10;
Su is a monosaccharide;
G is a monosaccharide moiety;
GlcNAc is an N-acetylglucosamine moiety;
Fuc is a fucose moiety;
d is 0 or 1.

19. A linker-drug construct according to structure (5):
wherein: 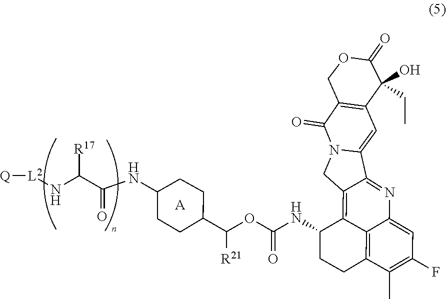
L² is a linker;
Q is a click probe capable of performing a metal-free click reaction or a thiol reactive probe;
each R¹⁷ is individually an amino acid side chain;
n is an integer in the range of 1-5;
A is a 5- or 6-membered aromatic or heteroaromatic ring;
x is an integer in the range of 1-8;
R²¹ is selected from H, R²², C(O)OH and C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups.

20. The linker-drug construct according to claim 19, wherein the click probe Q comprises a cyclic alkyne moiety or a cyclic alkene moiety.

21. The linker-drug construct according to claim 20, wherein:
(a) the click probe Q is selected from the group consisting of (Q22)-(Q36): 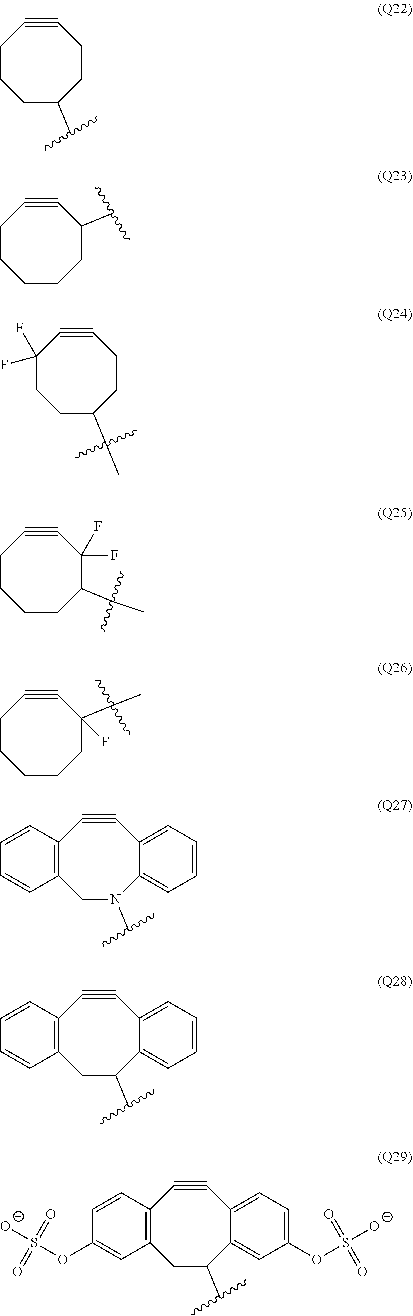 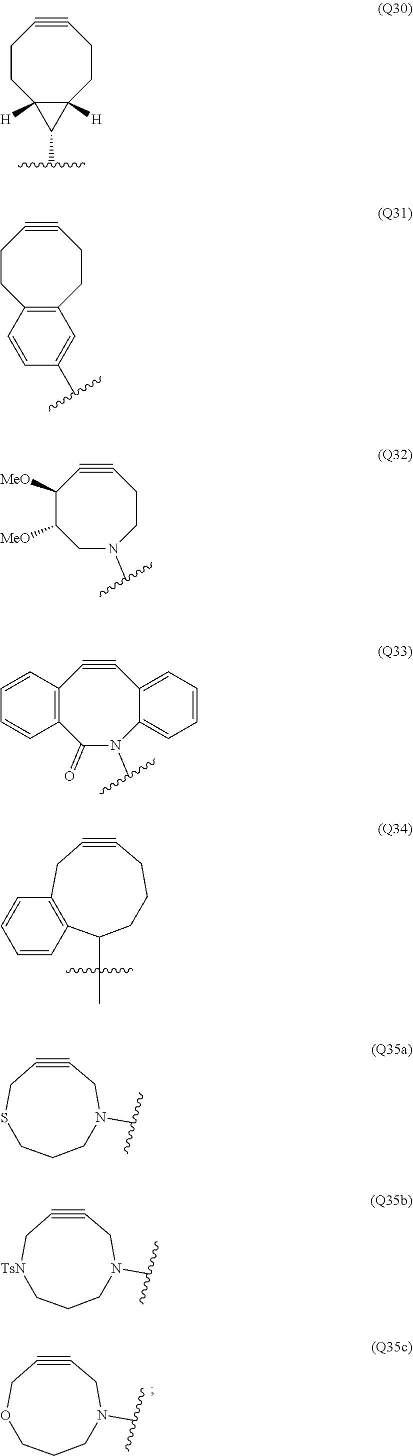 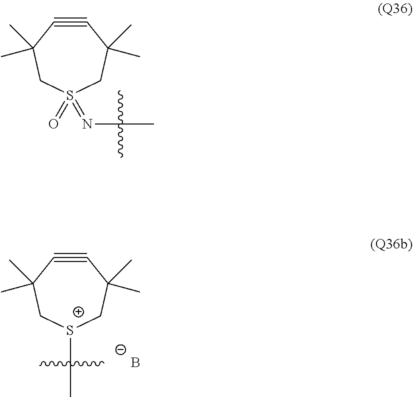
wherein B is a pharmaceutically acceptable anion;
or wherein the (hetero)cycloalkynyl moiety Q is according to structure (Q37): 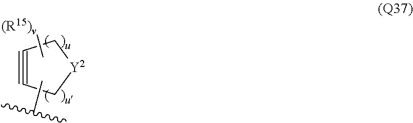
wherein:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, 07-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, 07-C₂₄ alkyl(hetero)aryl groups and C₇-C₂₄ (hetero)arylalkyl groups;
Y² is C(R³¹)₂, O, S or NR³¹, wherein each R³¹ individually is R¹⁵ or a second occurrence of the exatecan payload connected via a spacer moiety;
u is 0, 1, 2, 3, 4 or 5;
u′ is 0, 1, 2, 3, 4 or 5, wherein u+u′=4, 5, 6, 7 or 8;
v=an integer in the range 8-16.

22. The linker-drug construct according to claim 21, wherein the cyclooctynyl moiety Q is according to structure (Q38): 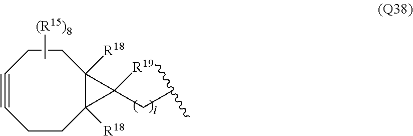
wherein
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, 07-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, 07-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of the exatecan payload connected via a spacer moiety; and
I is an integer in the range 0 to 10;
or wherein the (hetero)cyclooctynyl moiety Q is according to structure (Q39): 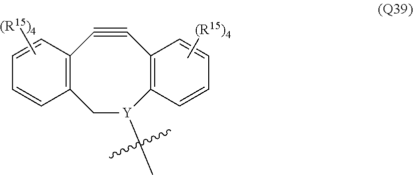
wherein
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵;
or wherein the heterocycloheptynyl moiety Q is according to structure (Q36a): 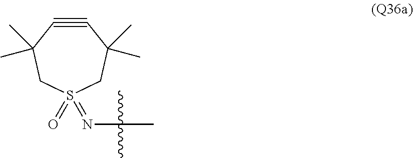
or (b) wherein the click probe Q is selected from the group consisting of, optionally substituted, (hetero)cyclopropenyl group, (hetero)cyclobutenyl group, trans-(hetero)cycloheptenyl group, trans-(hetero)cyclooctenyl group, trans-(hetero)cyclononenyl group or trans-(hetero)cyclodecynyl group.

23. The linker-drug construct according to claim 22, wherein click probe Q is selected from the group consisting of (Q40)-(Q50): 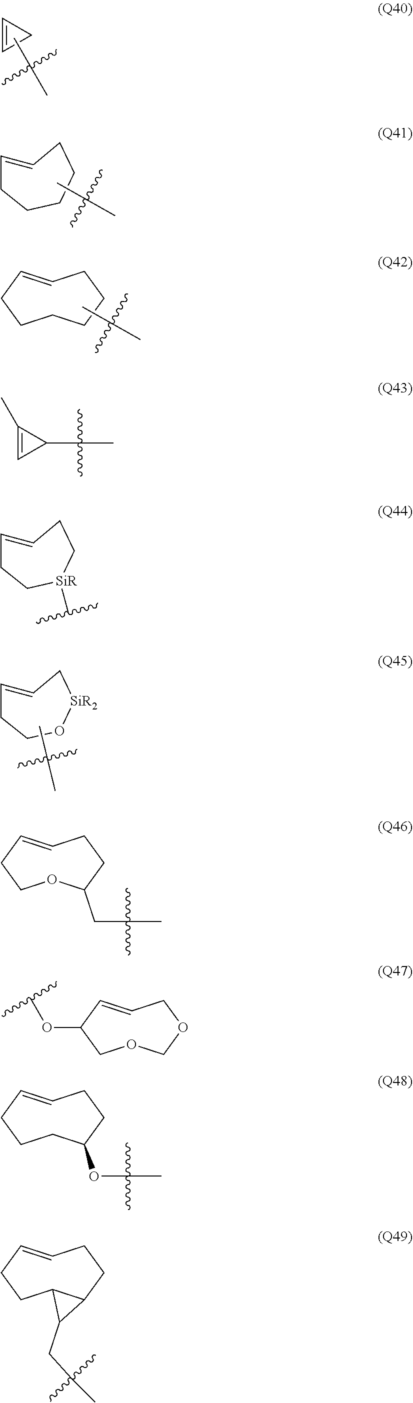 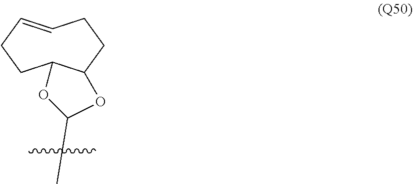 [SMILES: *C1OC2CC/C=C/CCC2O1]
wherein the R group(s) on Si in (Q44) and (Q45) is alkyl or aryl.

24. The linker-drug construct according to claim 19, wherein the thiol-reactive probe Q is selected from the group consisting of (Q51)-(Q65): 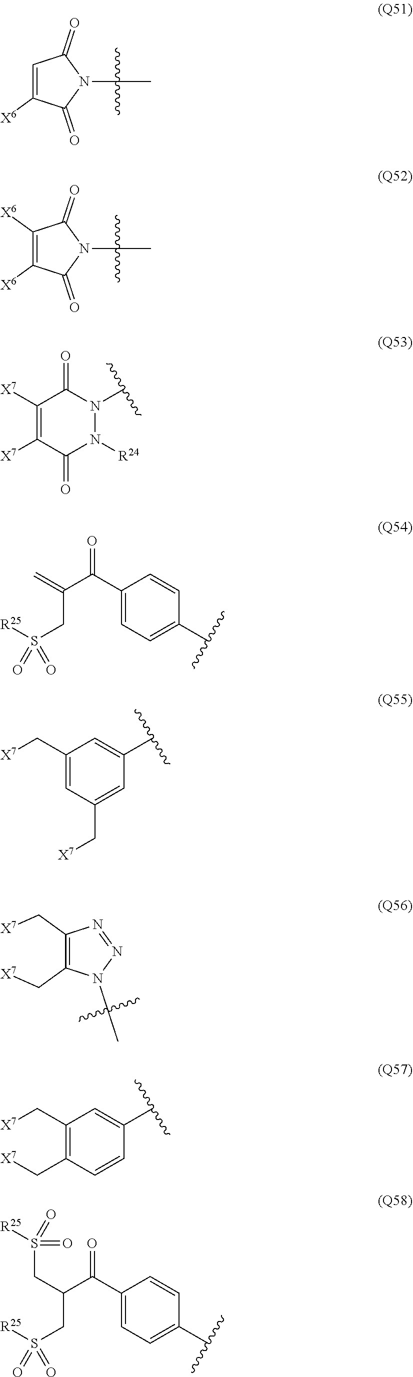 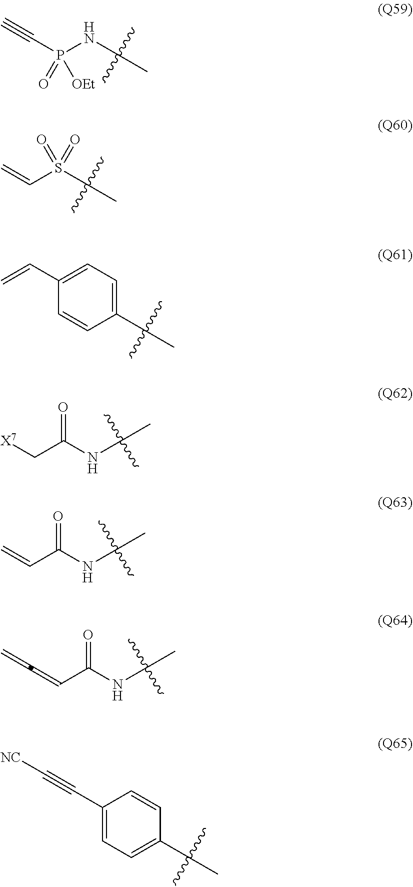
wherein:
X⁶ is H, halogen, PhS, MeS;
X⁷ is halogen, PhS, MeS;
R²⁴ is H or C₁₋₁₂ alkyl;
R²⁵ is H, C₁₋₁₂ alkyl, C₁₋₁₂ aryl, C₁₋₁₂ alkaryl or C₁₋₁₂ aralkyl;
wherein the aromatic ring of (Q55) and (Q57) may optionally be a heteroaromatic ring, such as a phenyl or pyridine ring.

25. The linker-drug construct according to claim 24, which has structure (5d) 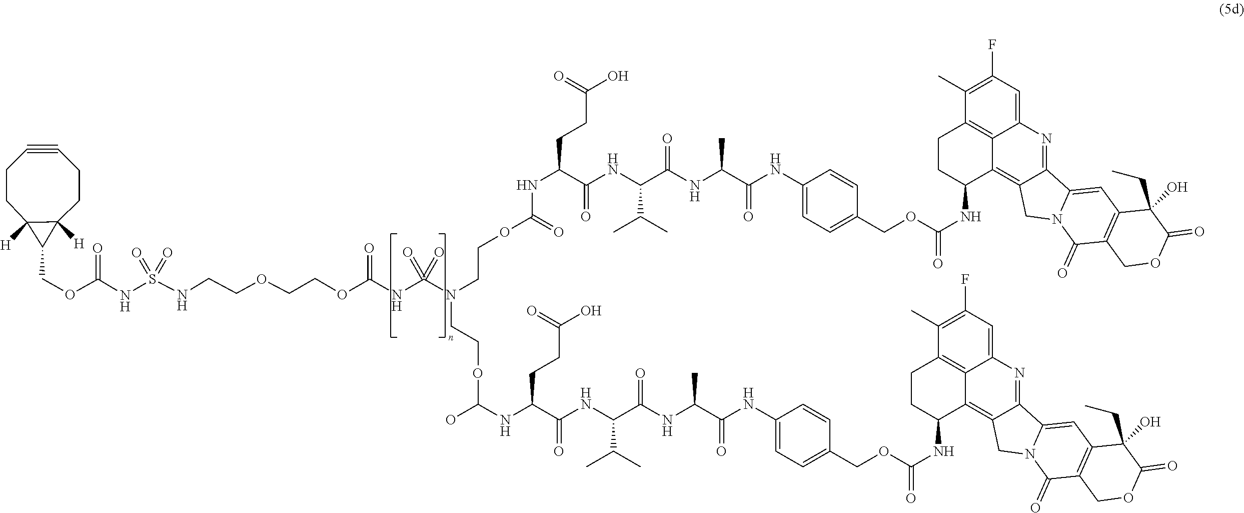
wherein n=0 or 1.

26. A pharmaceutical composition comprising the antibody-drug conjugate according to claim 1 and a pharmaceutically acceptable carrier.

27. A method of treating cancer, comprising administering to a patient in need thereof an antibody-drug conjugate according to claim 1.


SEQUENCE LISTING (1 sequence)

SEQ ID NO: 1 - protein, 4 aa, synthetic construct
  source 1..4: note = GGFG peptide
GGFG